(19) United States
(12) Patent Application Publication
(10) Pub. No.: US 2023/0287515 A1
(43) Pub. Date: Sep. 14, 2023

(54) COMPOSITIONS AND METHODS FOR DETECTING FUSION GENES

(71) Applicant: Singular Genomics Systems, Inc., San Diego, CA (US)

(72) Inventor: Timothy LOONEY, Austin, TX (US)

(21) Appl. No.: 18/183,818

(22) Filed: Mar. 14, 2023

Related U.S. Application Data
(60) Provisional application No. 63/356,901, filed on Jun. 29, 2022.
(60) Provisional application No. 63/348,947, filed on Jun. 3, 2022.
(60) Provisional application No. 63/319,521, filed on Mar. 14, 2022.

(51) Int. Cl. C12Q 1/6886

(52) U.S. Cl. CPC C12Q 1/6886; C12Q 2600/156

(57) ABSTRACT

Disclosed herein, inter alia, are compositions and methods providing sequencing-efficient solutions for detecting genetic features and aberrations in cancer.

### CROSS-REFERENCE TO RELATED APPLICATIONS

This application claims the benefit of U.S. Provisional Application No. 63/319,521, filed Mar. 14, 2022; U.S. Provisional Application No. 63/348,947, filed Jun. 3, 2022; and U.S. Provisional Application No. 63/356,901, filed Jun. 29, 2022; each of which are incorporated herein by reference in their entirety and for all purposes.

### SEQUENCE LISTING

The Sequence Listing titled, 051385-568001US SEQUENCE LISTING ST26.xml, was created on Mar. 8, 2023 in machine format IBM-PC, MS-Windows operation system and is 2,036 bytes, is hereby incorporated by reference in its entirety for all purposes.

### BACKGROUND

Gene fusions are a type of somatic alteration that can lead to cancer. Translocations, copy number changes, and inversions can lead to gene fusions, as well as dysregulated gene expression and novel molecular functions. Next generation sequencing (NGS) approaches for gene fusion detection may employ untargeted sequencing (e.g., whole genome or whole transcriptome sequencing) or targeted sequencing of fusion genes of interest. Targeted approaches for gene fusion detection enable simplified analysis and reduced cost. Popular methods for targeted sequencing of gene fusions include multiplex PCR, where primer sets are designed to generate PCR amplicons spanning known breakpoint junctions; anchored multiplex PCR (AMP); and methods utilizing hybridization capture to enrich for breakpoint regions of interest. However, multiplex PCR cannot identify fusions involving novel breakpoints and partners; AMP has a relatively higher input requirement and more complex workflow that is generally restricted to the analysis of RNA; and hybrid capture has a relatively complex workflow and reduced sensitivity compared to PCR based approaches. For both targeted and untargeted approaches, robustness to sample degradation is often of paramount importance owing to the widespread use of FFPE preserved tissue and cfDNA as input material.

### BRIEF SUMMARY

In view of the foregoing, there exists a need for methods to enable high sensitivity targeted analysis of gene fusions, with minimal workflow complexity and input requirement, and a robustness to highly degraded materials. Described herein, inter alia, are solutions to these and other problems in the art.

In an aspect is provided a method for detecting a fusion gene in a sample from a subject, the method including: a) circularizing a plurality of linear nucleic acid molecules of the sample to form a plurality of circular template polynucleotides, wherein one or more of the linear nucleic acid molecules include a fusion gene, thereby forming one or more fusion gene circular template polynucleotides; b) hybridizing a first primer and a second primer to the one or more fusion circular template polynucleotides and extending with a polymerase to generate fusion polynucleotide amplification products; and c) detecting the fusion polynucleotide amplification products, wherein detecting includes hybridizing one or more sequencing primers to the fusion polynucleotide amplification products and sequencing the fusion polynucleotide amplification products.

### BRIEF DESCRIPTION OF THE DRAWINGS

FIG. 1 illustrates outward facing primers (illustrated as the arrows) which are designed to target the region adjacent to a breakpoint location of interest in a fusion partner of interest.

FIGS. 2A-2C illustrates an inverse PCR approach. FIG. 2A illustrates an approach, consisting of (a) an outward facing inverse PCR primer pair and (b) an optional 5′ blocking oligomer which selectively binds to the unrearranged template adjacent to the inverse PCR primer pair and upstream of the expected fusion breakpoint region. Relative positions of the optional 5′ blocking oligomer is indicated within the diagram. A 5′ blocking oligomer refers to an oligonucleotide that binds on the 5′ side of the exon junction. In embodiments, and under suitable conditions, the optional 5′ blocking oligomer is not bound, enabling amplification of circularized template (e.g., cDNA contains a fusion junction). FIG. 2B illustrates in detail an embodiment showing the outward facing primers, which contain a target specific sequence (A), and optionally, a sequence for downstream library preparation and analysis (B). FIG. 2C illustrates two primers of an outward facing primer pair (i.e., primer A and primer B) binding to a known sequence of a template polynucleotide. Each primer of an outward facing primer pair may bind to a portion of the entire known sequence of the template polynucleotide.

FIG. 3 illustrates the strategy of FIG. 1 as applied to a fusion containing template (i.e., a polynucleotide containing a sequence of a first region fused to a sequence of a second region at a fusion junction). The optional 5′ blocking oligomer does not bind adjacent to the outward facing primers, permitting selective amplification of the junction containing templates from fragmented material. A 5′ blocking oligomer refers to an oligonucleotide that binds on the 5′ side of the exon junction. In embodiments, and under suitable conditions, the optional 5′ blocking oligomer prevents amplification of unrearranged templates (e.g., cDNA not containing a fusion junction).

FIG. 4 illustrates a circularized template containing a fusion junction. In embodiments, the circularized template contains two junctions: 1) a junction derived from the sample fusion and 2) a junction derived from circularization of the 5′ and 3′ ends of the linear nucleic acid molecule. In embodiments, the latter (i.e., junction derived from circularization) may be used to quantify and estimate template abundance and/or perform error correction.

FIG. 5 illustrates an exemplary overview for detecting a translocation. Following amplification and sequencing, the sequencing reads are mapped to a reference. A translocation event may give rise to an excess of intergenically-mapped sequences that align in part to the untargeted 5′ fusion gene (Gene A) and the targeted fusion partner (Gene B) proximal to the breakpoint.

FIG. 6 illustrates a bioinformatics workflow for breakpoint mapping. Briefly, sequencing reads from the target of interest are identified, for example, by k-mer matching or alignment. Circularization junctions are then identified by k-mer matching or alignment. In some embodiments, k-mer matching may be accomplished using a k-mer index reflecting circularization junctions of nucleic acids derived from known fusions. Next, a read is classified as having an intragenic or intergenic junction and the mapping location and density of mapped reads is determined. Direct alignment of reads to a breakpoint is not required but may facilitate analysis.

FIG. 7 illustrates an embodiment of the methods described herein applied to the analysis of IGH V(D)J rearrangements. (A) Traditional approaches to amplify IGH rearrangements involve multiplex PCR primers targeting the variable gene framework regions in conjunction with one or more joining gene primers. Such approaches are limited by the need for complex primer pools, an inability to detect rearrangements having somatic hypermutation within the primer binding sites, and an inability to identify translocations involving IGHJ genes. (B) By contrast, inverse PCR of the IGH locus utilizes outward facing primers targeting the rarely mutated joining gene region. The method minimizes the number of required primers, avoids dropout owing to somatic hypermutation, enables detection of IGHJ translocations, and permits estimation of template copy number via analysis of circularization junctions. Inclusion of an optional blocking element increases the fraction of rearrangement containing amplicons, facilitating downstream sequencing analysis. Amplification of an unrearranged template may also be performed without the blocking element.

FIG. 8 illustrates an embodiment of a design strategy for the methods described herein applied to IGH rearrangements. Outward facing primers are designed to amplify each IGHJ gene, while optional blocking oligomers target the region upstream and adjacent to each joining gene.

FIG. 9 illustrates an embodiment of a workflow for the analysis of B cell rearrangements via the methods described herein. Amplification of the IGH, IGK and IGL loci is followed by next generation sequencing. Resultant reads are filtered to remove short and off-target products, the circularization junction is identified, unique sequences are collapsed, then annotated for the presence of V(D)J rearrangements via IgBLAST or similar tool. Reads having a valid V(D)J rearrangement are used to determine the frequency and template counts for each rearrangement and to identify clonal rearrangements consistent with the presence of a B cell malignancy. Reads lacking a V(D)J rearrangement are assessed for the presence of translocations using k-mer analysis or methods known in the art (e.g., GeneFuse). A final report is produced indicating the V(D)J clonality of the sample and translocation status.

FIG. 10 illustrates a chart highlighting the temporal aspects of monitoring measurable residual disease (MRD) for acute lymphoblastic leukemia (ALL). Each line represents the level of residual disease over time for a different hypothetical patient following therapeutic intervention (e.g., radiation and/or chemotherapy) at various time points for post-treatment monitoring. The response curves include: DP (disease persistence), VEP (very early relapse), ER (early relapse), LR (late relapse), VLR (very late relapse), and NR (no relapse). 10⁻² is denoted as the proportion of leukemic cells which represents the approximate lower limit of detection for VER.

FIG. 11 illustrates outward facing primers (illustrated as the pair of arrows pointing away from each other) which are designed to target the region adjacent to a breakpoint location of interest in a fusion partner of interest are used in conjunction with inward facing primers (illustrated as the pair of arrows point towards each other) which are designed to target somatic mutations (e.g., single-nucleotide polymorphisms (SNP), insertions, deletions, copy number variations (CNV), etc.).

FIGS. 12A-12C illustrate amplification of a region of interest (e.g., either a single region of interest or a tandem duplication of a region of interest) using a single pooled multiplex amplification reaction (e.g., a single pooled multiplexed PCR reaction). FIG. 12A illustrates an embodiment wherein two pairs of overlapping inward facing primers (e.g., 1F and 1R, and 2F and 2R) are used to amplify a target region, resulting in three amplification products (e.g., three PCR products: Amplicon 1 (amplification product of the 1F and 1R primer pair), Amplicon 2 (amplification product of the 2F and 2R primer pair), and a Maxi-Amplicon (amplification product of the 1F and 2R primer pair), as described in U.S. Pat. Pub. US2016/0340746, which is incorporated herein by reference in its entirety. Production of a Mini-Amplicon by the 2F and 1R primer pair is suppressed due to stable secondary structure resulting in less efficient amplification. The products of the amplification reaction with the overlapping inward facing primers are identical whether a linear or circularized template is used. FIG. 12B illustrates the expected amplification products from an embodiment wherein amplification of an internal tandem duplication is performed with the primer pairs of FIG. 12A (e.g., 1F and 1R, and 2F and 2R) when using a linear template. The amplification products are identical to those of the non-duplicated template in FIG. 12A (e.g., Amplicon 1, Amplicon 2, and the Maxi-Amplicon), precluding detection of the tandem duplication event. FIG. 12C illustrates the expected amplification products from an embodiment wherein amplification of an internal tandem duplication is performed with the primer pairs of FIG. 12A (e.g., 1F and 1R, and 2F and 2R) when using a circularized template. The amplification products now include a duplication-specific amplicon (e.g., an amplification product of the 2R and 1F primer pair). The duplication-specific amplicon is identified both by the unique pair of primers appearing in the amplicon and the presence of a circularization junction within the amplicon (denoted by the dashed line).

FIG. 13 illustrates the blocking element efficiency as determined by gel electrophoresis analysis. Synthetic oligomers were produced to represent an IGH rearrangement (Fusion, F) and an unrearranged IGHJ6 gene (Wild Type, W). PCR amplification of each template was conducted using inverse PCR primers in the presence or absence of a non-extendable blocking oligomer (denoted by +/−) capable of hybridizing to the W template but not the F template (as illustrated in FIG. 2A including the optional blocking element). Arrow indicates location of expected product. PCR amplification products were then visualized on an agarose gel.

FIG. 14 shows the results of a bioinformatic reconstruction of a detected breakpoint region within the BCL2 locus of chromosome 18 using the methods described herein. Each grey horizontal line represents a sequenced fragment, and a visual representation of the coverage is represented on the top.

FIG. 15 outlines a preferred method described herein. For example, the method utilizes inverse PCR for targeted detection of gene fusions with novel partners.

### DETAILED DESCRIPTION

Described herein are novel methods for detecting gene fusions within and across different, independent chromosomes.

### I. Definitions

The practice of the technology described herein will employ, unless indicated specifically to the contrary, conventional methods of chemistry, biochemistry, organic chemistry, molecular biology, microbiology, recombinant DNA techniques, genetics, immunology, and cell biology that are within the skill of the art, many of which are described below for the purpose of illustration. Examples of such techniques are available in the literature. Methods, devices and materials similar or equivalent to those described herein can be used in the practice of this invention.

All patents, patent applications, articles and publications mentioned herein, both supra and infra, are hereby expressly incorporated herein by reference in their entireties.

Unless defined otherwise herein, all technical and scientific terms used herein have the same meaning as commonly understood by one of ordinary skill in the art to which this disclosure belongs. Various scientific dictionaries that include the terms included herein are well known and available to those in the art. Although any methods and materials similar or equivalent to those described herein find use in the practice or testing of the disclosure, some preferred methods and materials are described. Accordingly, the terms defined immediately below are more fully described by reference to the specification as a whole. It is to be understood that this disclosure is not limited to the particular methodology, protocols, and reagents described, as these may vary, depending upon the context in which they are used by those of skill in the art. The following definitions are provided to facilitate understanding of certain terms used frequently herein and are not meant to limit the scope of the present disclosure.

As used herein, the singular terms “a”, “an”, and “the” include the plural reference unless the context clearly indicates otherwise. Reference throughout this specification to, for example, “one embodiment”, “an embodiment”, “another embodiment”, “a particular embodiment”, “a related embodiment”, “a certain embodiment”, “an additional embodiment”, or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

As used herein, the term “control” or “control experiment” is used in accordance with its plain and ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects.

As used herein, the term “complement” is used in accordance with its plain and ordinary meaning and refers to a nucleotide (e.g., RNA nucleotide or DNA nucleotide) or a sequence of nucleotides capable of base pairing with a complementary nucleotide or sequence of nucleotides. As described herein and commonly known in the art the complementary (matching) nucleotide of adenosine is thymidine in DNA, or alternatively in RNA the complementary (matching) nucleotide of adenosine is uracil, and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence. “Duplex” means at least two oligonucleotides and/or polynucleotides that are fully or partially complementary undergo Watson-Crick type base pairing among all or most of their nucleotides so that a stable complex is formed.

As described herein, the complementarity of sequences may be partial, in which only some of the nucleic acids match according to base pairing, or complete, where all the nucleic acids match according to base pairing. Thus, two sequences that are complementary to each other, may have a specified percentage of nucleotides that complement one another (e.g., about 60%, preferably 65%, 70%, 75%, 80%, 85%, 90%, 91%, 92%, 93%, 94%, 95%, 96%, 97%, 98%, 99%, or higher complementarity over a specified region). In embodiments, two sequences are complementary when they are completely complementary, having 100% complementarity. In embodiments, sequences in a pair of complementary sequences form portions of a single polynucleotide with non-base-pairing nucleotides (e.g., as in a hairpin structure, with or without an overhang) or portions of separate polynucleotides. In embodiments, one or both sequences in a pair of complementary sequences form portions of longer polynucleotides, which may or may not include additional regions of complementarity.

As used herein, the term “contacting” is used in accordance with its plain ordinary meaning and refers to the process of allowing at least two distinct species (e.g., chemical compounds including biomolecules or cells) to become sufficiently proximal to react, interact or physically touch. However, the resulting reaction product can be produced directly from a reaction between the added reagents or from an intermediate from one or more of the added reagents that can be produced in the reaction mixture. The term “contacting” may include allowing two species to react, interact, or physically touch, wherein the two species may be a compound, nucleic acid, a protein, or enzyme (e.g., a DNA polymerase).

As used herein, the term “nucleic acid” is used in accordance with its plain and ordinary meaning and refers to nucleotides (e.g., deoxyribonucleotides or ribonucleotides) and polymers thereof in either single-, double- or multiple-stranded form, or complements thereof. The terms “polynucleotide,” “oligonucleotide,” “oligo”, “oligomer” or the like refer, in the usual and customary sense, to a sequence of nucleotides. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA with linear or circular framework. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may include natural nucleic acid sequences and variants thereof, artificial nucleic acid sequences, or a combination of such sequences. A “nucleoside” is structurally similar to a nucleotide, but is missing the phosphate moieties. An example of a nucleoside analogue would be one in which the label is linked to the base and there is no phosphate group attached to the sugar molecule. As may be used herein, the terms “nucleic acid oligomer” and “oligonucleotide” are used interchangeably and are intended to include, but are not limited to, nucleic acids having a length of 200 nucleotides or less. In some embodiments, an oligonucleotide is a nucleic acid having a length of 2 to 200 nucleotides, 2 to 150 nucleotides, 5 to 150 nucleotides or 5 to 100 nucleotides. A “linear nucleic acid molecule” is nucleic acid (e.g. oligomer or polymer) in which that 5′ and 3′ termini are not joined. A “circular” nucleic acid (e.g. a “circular template polynucleotide”) is a nucleic in which the 5′ and 3′ termini are joined together.

The term “primer,” as used herein, is defined to be one or more nucleic acid fragments that may specifically hybridize to a nucleic acid template, be bound by a polymerase, and be extended in a template-directed process for nucleic acid synthesis. A primer can be of any length depending on the particular technique it will be used for. For example, PCR primers are generally between 10 and 40 nucleotides in length. In some embodiments, a primer has a length of 200 nucleotides or less. In certain embodiments, a primer has a length of 10 to 150 nucleotides, 15 to 150 nucleotides, 5 to 100 nucleotides, 5 to 50 nucleotides or 10 to 50 nucleotides. The length and complexity of the nucleic acid fixed onto the nucleic acid template is not critical. One of skill can adjust these factors to provide optimum hybridization and signal production for a given hybridization procedure, and to provide the desired resolution among different genes or genomic locations. The primer permits the addition of a nucleotide residue thereto, or oligonucleotide or polynucleotide synthesis therefrom, under suitable conditions known in the art. In an embodiment the primer is a DNA primer, i.e., a primer consisting of, or largely consisting of, deoxyribonucleotide residues. The primers are designed to have a sequence that is the complement of a region of template/target DNA to which the primer hybridizes. The addition of a nucleotide residue to the 3′ end of a primer by formation of a phosphodiester bond results in a DNA extension product. The addition of a nucleotide residue to the 3′ end of the DNA extension product by formation of a phosphodiester bond results in a further DNA extension product. In another embodiment the primer is an RNA primer. In embodiments, a primer is hybridized to a target polynucleotide. A “primer” includes a sequence that is complementary to a polynucleotide template, and complexes by hydrogen bonding or hybridization with the template to give a primer/template complex for initiation of synthesis by a polymerase, which is extended by the addition of covalently bonded bases linked at its 3′ end complementary to the template in the process of DNA synthesis.

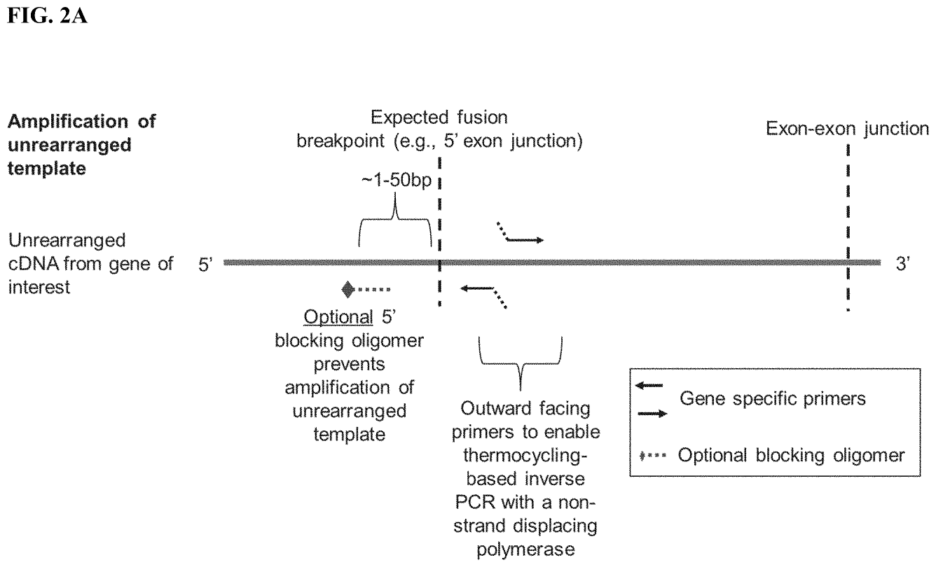

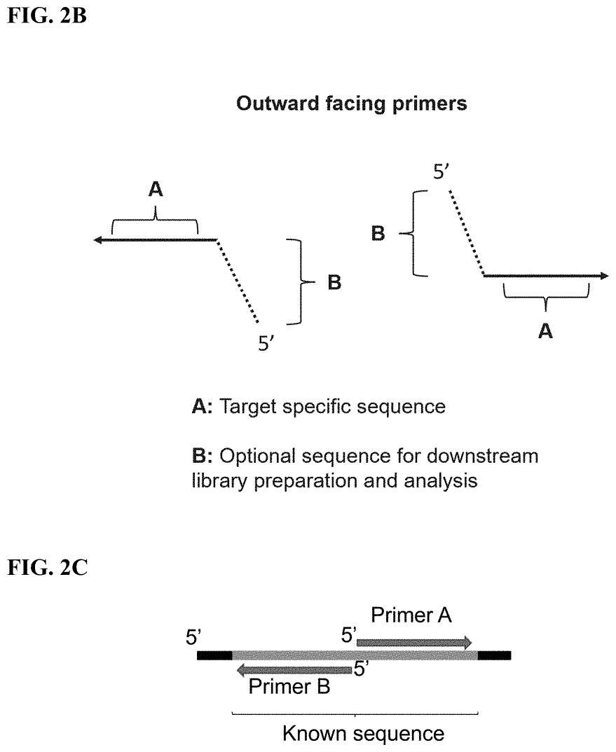

The term “inward facing primers” refers to a pair of primer oligonucleotides (e.g., a forward and reverse), wherein one primer is complementary to a first polynucleotide (i.e., the top strand), and a second primer is complementary to the complement of the first polynucleotide (i.e., the bottom strand). Inward facing primers bind on opposite ends of a linear dsDNA molecule, wherein the sequence to be amplified (and the complement thereof) is between the primer binding regions. Inward facing primers are commonly illustrated with their 3′-OH ends pointing towards each other. For example, a forward primer anneals to the bottom strand that runs 3′ to 5′, and the reverse primer anneals to the complementary bottom strand of DNA that runs 5′ to 3′. After extension, this results in two new strands of double-stranded DNA: one made from the forward primer; and the other made from the reverse primer. Inward facing primers may exponentially amplify a target DNA molecule if the target DNA molecule is in either linear or circular form. In contrast, outward facing primers only exponentially amplify a target polynucleotide molecule if the target polynucleotide molecule is in circular form. Outward facing primers are commonly illustrated with their 3′-OH ends pointing away from each other. The term “outward facing primers” refers to a pair of primer oligonucleotides that are inverted relative to conventional inward facing primers and will amplify around the circularized template polynucleotide. Each primer of the outward facing primer pair binds to a different circular DNA polynucleotide, and they both point away into the unknown DNA, for example illustrated in FIG. 2C. A pair of outward facing primers may include a pair of primer including sequences which lie end to end on a known sequence. In embodiments, the pair of outward facing primers are complementary to the same primer binding sequence, wherein one primer of the pair binds to a portion of the primer binding sequence and a second primer of the paid binds to a portion of the primer binding sequence. In embodiments, the first primer of an outward facing primer set binds 1 to 5 nucleotides upstream of the second primer of an outward facing primer set. In embodiments, the first primer of an outward facing primer set binds 1 to 5 nucleotides downstream of the second primer of an outward facing primer set. To prevent primer-dimer artefacts, however it is preferred to make the outward facing primers not substantially complementary to each other. For example, using the sense strand of the target region as reference and the 5′ and 3′ ends of that sense strand as “upstream” and “downstream” reference points, respectively, outward PCR primers are designed such that a first primer is complementary to a region of the sense strand, a second primer is complementary to a region of the antisense strand, and the region complementary to the first primer is upstream in relation to the region complementary to the second primer. Outward facing primers are useful for amplifying sequences that flank one end of a known DNA sequence. That is, the unknown sequence is amplified by two outward facing primers that bind to the known sequence and point in opposite directions of a circular polynucleotide.

The term “inverse PCR” is used in accordance with its ordinary meaning in the art and refers to a method of polymerase chain reaction (PCR) which utilizes outward facing primers to amplify a polynucleotide. In contrast, traditional PCR utilizes inward facing primers, that is, the primers are oriented such that extension proceeds inwards across the region between the two primers.

As used herein, the terms “solid support” and “substrate” and “solid surface” refers to discrete solid or semi-solid surfaces to which a plurality of primers may be attached. A solid support may encompass any type of solid, porous, or hollow sphere, ball, cylinder, or other similar configuration composed of plastic, ceramic, metal, or polymeric material (e.g., hydrogel) onto which a nucleic acid may be immobilized (e.g., covalently or non-covalently). A solid support may include a discrete particle that may be spherical (e.g., microspheres) or have a non-spherical or irregular shape, such as cubic, cuboid, pyramidal, cylindrical, conical, oblong, or disc-shaped, and the like. Solid supports in the form of discrete particles may be referred to herein as “beads,” which alone does not imply or require any particular shape. A bead can be non-spherical in shape. A solid support may further include a polymer or hydrogel on the surface to which the primers are attached (e.g., the splint primers are covalently attached to the polymer, wherein the polymer is in direct contact with the solid support). Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefin copolymers, polyimides etc.), nylon, ceramics, resins, Zeonor, silica or silica-based materials including silicon and modified silicon, carbon, metals, inorganic glasses, optical fiber bundles, photopatternable dry film resists, UV-cured adhesives and polymers. The solid supports for some embodiments have at least one surface located within a flow cell. The solid support, or regions thereof, can be substantially flat. The solid support can have surface features such as wells, pits, channels, ridges, raised regions, pegs, posts or the like. The term solid support is encompassing of a substrate (e.g., a flow cell) having a surface including a polymer coating covalently attached thereto. In embodiments, the solid support is a flow cell. The term “flow cell” as used herein refers to a chamber including a solid surface across which one or more fluid reagents can be flowed. Examples of flow cells and related fluidic systems and detection platforms that can be readily used in the methods of the present disclosure are described, for example, in Bentley et al., Nature. 456:53-59 (2008).

In some embodiments, a nucleic acid includes a capture nucleic acid. A capture nucleic acid refers to a nucleic acid that is attached to a substrate (e.g., covalently attached). In some embodiments, a capture nucleic acid includes a primer. In some embodiments, a capture nucleic acid is a nucleic acid configured to specifically hybridize to a portion of one or more nucleic acid templates (e.g., a template of a library). In some embodiments, a capture nucleic acid configured to specifically hybridize to a portion of one or more nucleic acid templates is substantially complementary to a suitable portion of a nucleic acid template, or an amplicon thereof. In some embodiments, a capture nucleic acid is configured to specifically hybridize to a portion of an adapter, or a portion thereof. In some embodiments a capture nucleic acid, or portion thereof, is substantially complementary to a portion of an adapter, or a complement thereof. In embodiments, a capture nucleic acid is a probe oligonucleotide. Typically, a probe oligonucleotide is complementary to a target polynucleotide or portion thereof, and further includes a label (such as a binding moiety) or is attached to a surface, such that hybridization to the probe oligonucleotide permits the selective isolation of probe-bound polynucleotides from unbound polynucleotides in a population. A probe oligonucleotide may or may not also be used as a primer.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amio acid on a protein or polypeptide through a covalent, non-covalent, or other interaction.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

As used herein, the term “template nucleic acid” refers to any polynucleotide molecule that may be bound by a polymerase and utilized as a template for nucleic acid synthesis. A template nucleic acid may be a target nucleic acid. In general, the term “target nucleic acid” refers to a nucleic acid molecule or polynucleotide in a starting population of nucleic acid molecules having a target sequence whose presence, amount, and/or nucleotide sequence, or changes in one or more of these, are desired to be determined. In general, the term “target sequence” refers to a nucleic acid sequence on a single strand of nucleic acid. The target sequence may be a portion of a gene, a regulatory sequence, genomic DNA, cDNA, RNA including mRNA, miRNA, rRNA, or others. The target sequence may be a target sequence from a sample or a secondary target such as a product of an amplification reaction. A target nucleic acid is not necessarily any single molecule or sequence. For example, a target nucleic acid may be any one of a plurality of target nucleic acids in a reaction, or all nucleic acids in a given reaction, depending on the reaction conditions. For example, in a nucleic acid amplification reaction with random primers, all polynucleotides in a reaction may be amplified. As a further example, a collection of targets may be simultaneously assayed using polynucleotide primers directed to a plurality of targets in a single reaction. As yet another example, all or a subset of polynucleotides in a sample may be modified by the addition of a primer-binding sequence (such as by the ligation of adapters containing the primer binding sequence), rendering each modified polynucleotide a target nucleic acid in a reaction with the corresponding primer polynucleotide(s). In the context of selective sequencing, “target nucleic acid(s)” refers to the subset of nucleic acid(s) to be sequenced from within a starting population of nucleic acids.

The term “polynucleotide fusion” is used in accordance with its plain and ordinary meaning and refers to a polynucleotide formed from the joining of two regions of a reference sequence (e.g., a reference genome) that are not so joined in the reference sequence, thereby creating a fusion junction between the two regions that does not exist in the reference sequence. Polynucleotide fusions can be formed by a number of processes, including interchromosomal translocation, intrachromosomal translocation, and other chromosomal rearrangements (e.g., inversion and duplication). A polynucleotide fusion can involve fusion between two gene sequences, referred to as a “gene fusion” and producing a “fusion gene.” In some cases, a fusion gene is expressed as a fusion transcript (e.g., a fusion mRNA transcript) including sequences of the two genes, or portions thereof.

A “fusion gene” is used in accordance with its ordinary meaning in the art and refers to a hybrid gene, or portion thereof, formed from two previously independent genes, or portions thereof (e.g., in a cell). A “fusion junction” is the point in the fusion gene sequence between the two previously independent genes, or portions thereof. The hybrid gene can result from a translocation, interstitial deletion, and/or chromosomal inversion of a gene or portion of a gene. Chromosomal rearrangements leading to the fusion of coding regions of two genes can result in expression of hybrid proteins. An “exon junction” is the point or location in the fusion gene sequence between the two previously independent exon sequences, or portions thereof.

A nucleic acid can be amplified by a suitable method. The term “amplified” as used herein refers to subjecting a target nucleic acid in a sample to a process that linearly or exponentially generates amplicon nucleic acids having the same or substantially the same (e.g., substantially identical) nucleotide sequence as the target nucleic acid, or segment thereof, and/or a complement thereof. In some embodiments an amplification reaction includes a suitable thermal stable polymerase. Thermal stable polymerases are known in the art and are stable for prolonged periods of time, at temperature greater than 80° C. when compared to common polymerases found in most mammals. In certain embodiments the term “amplified” refers to a method that includes a polymerase chain reaction (PCR). Conditions conducive to amplification (i.e., amplification conditions) often include at least a suitable polymerase, a suitable template, a suitable primer or set of primers, suitable nucleotides (e.g., dNTPs), a suitable buffer, and application of suitable annealing, hybridization and/or extension times and temperatures. In certain embodiments an amplified product (e.g., an amplicon) can contain one or more additional and/or different nucleotides than the template sequence, or portion thereof, from which the amplicon was generated (e.g., a primer can contain “extra” nucleotides (such as a 5′ portion that does not hybridize to the template), or one or more mismatched bases within a hybridizing portion of the primer).

As used herein, “differential amplification” or “differentially amplifying” refers to amplification of a gene of interest to a greater degree than amplification of a reference gene thereby resulting in a greater number of amplification products from the gene of interest relative to the number of amplification products from the reference gene. In embodiments, the gene of interest includes a polynucleotide sequence including a fusion gene and the gene of interest includes a polynucleotide not including the fusion gene.

As used herein, the term “ligase” refers to an enzyme that catalyzes the formation of a new phosphodiester bond as a result of joining the 5′-phosphoryl terminus of DNA or RNA to single-stranded 3′-hydroxyl terminus of DNA or RNA. Ligase enzymes can form circular DNA or RNA templates in a non-template driven reaction, and examples of ligase enzymes include, but are not limited to, as CircLigase, Taq DNA Ligase, HiFi Taq DNA Ligase, T4 ligase, or Ampligase® DNA Ligase.

As used herein, the term “rolling circle amplification (RCA)” refers to a nucleic acid amplification reaction that amplifies a circular nucleic acid template (e.g., single-stranded DNA circles) via a rolling circle mechanism. Rolling circle amplification reaction is initiated by the hybridization of a primer to a circular, often single-stranded, nucleic acid template. The nucleic acid polymerase then extends the primer that is hybridized to the circular nucleic acid template by continuously progressing around the circular nucleic acid template to replicate the sequence of the nucleic acid template over and over again (rolling circle mechanism). The rolling circle amplification typically produces concatemers including tandem repeat units of the circular nucleic acid template sequence. The rolling circle amplification may be a linear RCA (LRCA), exhibiting linear amplification kinetics (e.g., RCA using a single specific primer), or may be an exponential RCA (eRCA) exhibiting exponential amplification kinetics. Rolling circle amplification may also be performed using multiple primers (multiply primed rolling circle amplification or MPRCA) leading to hyper-branched concatemers. For example, in a double-primed RCA, one primer may be complementary, as in the linear RCA, to the circular nucleic acid template, whereas the other may be complementary to the tandem repeat unit nucleic acid sequences of the RCA product. Consequently, the double-primed RCA may proceed as a chain reaction with exponential (geometric) amplification kinetics featuring a ramifying cascade of multiple-hybridization, primer-extension, and strand-displacement events involving both the primers. This often generates a discrete set of concatemeric, double-stranded nucleic acid amplification products. The rolling circle amplification may be performed in vitro under isothermal conditions using a suitable nucleic acid polymerase such as Phi29 DNA polymerase. RCA may be performed by using any of the DNA polymerases that are known in the art (e.g., a Phi29 DNA polymerase, a Bst DNA polymerase, or SD polymerase).

A nucleic acid can be amplified by a thermocycling method or by an isothermal amplification method. In some embodiments, a rolling circle amplification method is used. In some embodiments, amplification takes place on a solid support (e.g., within a flow cell) where a nucleic acid, nucleic acid library or portion thereof is immobilized. In certain sequencing methods, a nucleic acid library is added to a flow cell and immobilized by hybridization to anchors under suitable conditions. This type of nucleic acid amplification is often referred to as solid phase amplification. In some embodiments of solid phase amplification, all or a portion of the amplified products are synthesized by an extension initiating from an immobilized primer. Solid phase amplification reactions are analogous to standard solution phase amplifications except that at least one of the amplification oligonucleotides (e.g., primers) is immobilized on a solid support.

In some embodiments, solid phase amplification includes a nucleic acid amplification reaction including only one species of oligonucleotide primer immobilized to a surface or substrate. In certain embodiments, solid phase amplification includes a plurality of different immobilized oligonucleotide primer species. In some embodiments, solid phase amplification may include a nucleic acid amplification reaction including one species of oligonucleotide primer immobilized on a solid surface and a second different oligonucleotide primer species in solution. Multiple different species of immobilized or solution-based primers can be used. Non-limiting examples of solid phase nucleic acid amplification reactions include interfacial amplification, bridge PCR amplification, emulsion PCR, WildFire amplification (e.g., US patent publication US20130012399), and the like, or combinations thereof.

In embodiments, a target nucleic acid is a cell-free nucleic acid. In general, the terms “cell-free,” “circulating,” and “extracellular” as applied to nucleic acids (e.g. “cell-free DNA” (cfDNA) and “cell-free RNA” (cfRNA)) are used interchangeably to refer to nucleic acids present in a sample from a subject or portion thereof that can be isolated or otherwise manipulated without applying a lysis step to the sample as originally collected (e.g., as in extraction from cells or viruses). Cell-free nucleic acids are thus unencapsulated or “free” from the cells or viruses from which they originate, even before a sample of the subject is collected. Cell-free nucleic acids may be produced as a byproduct of cell death (e.g., apoptosis or necrosis) or cell shedding, releasing nucleic acids into surrounding body fluids or into circulation. Accordingly, cell-free nucleic acids may be isolated from a non-cellular fraction of blood (e.g., serum or plasma), from other bodily fluids (e.g., urine), or from non-cellular fractions of other types of samples.

As used herein, the terms “analogue” and “analog”, in reference to a chemical compound, refers to compound having a structure similar to that of another one, but differing from it in respect of one or more different atoms, functional groups, or substructures that are replaced with one or more other atoms, functional groups, or substructures. In the context of a nucleotide, a “nucleotide analog” and “modified nucleotide” refer to a compound that, like the nucleotide of which it is an analog, can be incorporated into a nucleic acid molecule (e.g., an extension product) by a suitable polymerase, for example, a DNA polymerase in the context of a nucleotide analogue. The terms also encompass nucleic acids containing known nucleotide analogs or modified backbone residues or linkages, which are synthetic, naturally occurring, or non-naturally occurring, which have similar binding properties as the reference nucleic acid, and which are metabolized in a manner similar to the reference nucleotides. Examples of such analogs include, include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see, e.g., see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA)), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

As used herein, a “native” nucleotide is used in accordance with its plain and ordinary meaning and refers to a naturally occurring nucleotide that does not include an exogenous label (e.g., a fluorescent dye, or other label) or chemical modification such as those that may characterize a nucleotide analog (e.g., a reversible terminating moiety). Examples of native nucleotides useful for carrying out procedures described herein include: dATP (2′-deoxyadenosine-5′-triphosphate); dGTP (2′deoxyguanosine-5′-triphosphate); dCTP (2′-deoxycytidine-5′-triphosphate); dTTP (2′-deoxythymidine-5′-triphosphate); and dUTP (2′-deoxyuridine-5′-triphosphate).

As used herein, the term “modified nucleotide” refers to a nucleotide modified in some manner. Typically, a nucleotide contains a single 5-carbon sugar moiety, a single nitrogenous base moiety and 1 to three phosphate moieties. In embodiments, a nucleotide can include a blocking moiety (alternatively referred to herein as a reversible terminator moiety) and/or a label moiety. A blocking moiety on a nucleotide prevents formation of a covalent bond between the 3′ hydroxyl moiety of the nucleotide and the 5′ phosphate of another nucleotide. A blocking moiety on a nucleotide can be reversible, whereby the blocking moiety can be removed or modified to allow the 3′ hydroxyl to form a covalent bond with the 5′ phosphate of another nucleotide. A blocking moiety can be effectively irreversible under particular conditions used in a method set forth herein. In embodiments, the blocking moiety is attached to the 3′ oxygen of the nucleotide and is independently —NH₂, —CN, —CH₃, C₂-C₆ allyl (e.g., —CH₂—CH═CH₂), methoxyalkyl (e.g., —CH₂—O—CH₃), or —CH₂N₃. In embodiments, the blocking moiety is attached to the 3′ oxygen of the nucleotide and is independently

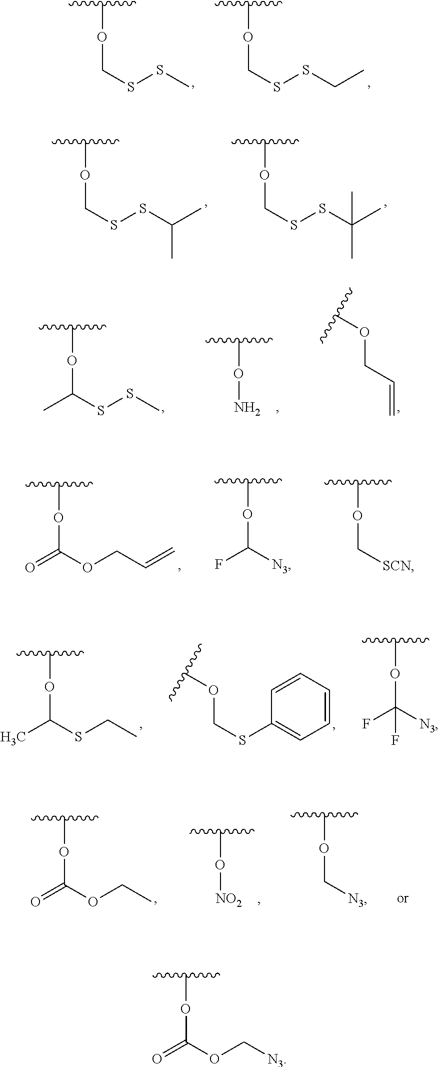

A label moiety of a nucleotide can be any moiety that allows the nucleotide to be detected, for example, using a spectroscopic method. Exemplary label moieties are fluorescent labels, mass labels, chemiluminescent labels, electrochemical labels, detectable labels and the like. One or more of the above moieties can be absent from a nucleotide used in the methods and compositions set forth herein. For example, a nucleotide can lack a label moiety or a blocking moiety or both. Examples of nucleotide analogues include, without limitation, 7-deaza-adenine, 7-deaza-guanine, the analogues of deoxynucleotides shown herein, analogues in which a label is attached through a cleavable linker to the 5-position of cytosine or thymine or to the 7-position of deaza-adenine or deaza-guanine, and analogues in which a small chemical moiety is used to cap the —OH group at the 3′-position of deoxyribose. Nucleotide analogues and DNA polymerase-based DNA sequencing are also described in U.S. Pat. No. 6,664,079, which is incorporated herein by reference in its entirety for all purposes.

In embodiments, the nucleotides of the present disclosure use a cleavable linker to attach the label to the nucleotide. The use of a cleavable linker ensures that the label can, if required, be removed after detection, avoiding any interfering signal with any labelled nucleotide incorporated subsequently. The use of the term “cleavable linker” is not meant to imply that the whole linker is required to be removed from the nucleotide base. The cleavage site can be located at a position on the linker that ensures that part of the linker remains attached to the nucleotide base after cleavage. The linker can be attached at any position on the nucleotide base provided that Watson-Crick base pairing can still be carried out. In the context of purine bases, it is preferred if the linker is attached via the 7-position of the purine or the preferred deazapurine analogue, via an 8-modified purine, via an N-6 modified adenosine or an N-2 modified guanine. For pyrimidines, attachment is preferably via the 5-position on cytidine, thymidine or uracil and the N-4 position on cytosine.

In embodiments, the nucleotides of the present disclosure use a cleavable linker to attach the label to the nucleotide. The use of a cleavable linker ensures that the label can, if required, be removed after detection, avoiding any interfering signal with any labelled nucleotide incorporated subsequently. The use of the term “cleavable linker” is not meant to imply that the whole linker is required to be removed from the nucleotide base. The cleavage site can be located at a position on the linker that ensures that part of the linker remains attached to the nucleotide base after cleavage. The linker can be attached at any position on the nucleotide base provided that Watson-Crick base pairing can still be carried out. In the context of purine bases, it is preferred if the linker is attached via the 7-position of the purine or the preferred deazapurine analogue, via an 8-modified purine, via an N-6 modified adenosine or an N-2 modified guanine. For pyrimidines, attachment is preferably via the 5-position on cytidine, thymidine or uracil and the N-4 position on cytosine. The term “cleavable linker” or “cleavable moiety” as used herein refers to a divalent or monovalent, respectively, moiety which is capable of being separated (e.g., detached, split, disconnected, hydrolyzed, a stable bond within the moiety is broken) into distinct entities. A cleavable linker is cleavable (e.g., specifically cleavable) in response to external stimuli (e.g., enzymes, nucleophilic/basic reagents, reducing agents, photo-irradiation, electrophilic/acidic reagents, organometallic and metal reagents, or oxidizing reagents). A chemically cleavable linker refers to a linker which is capable of being split in response to the presence of a chemical (e.g., acid, base, oxidizing agent, reducing agent, Pd(0), tris-(2-carboxyethyl)phosphine, dilute nitrous acid, fluoride, tris(3-hydroxypropyl)phosphine), sodium dithionite (Na₂S₂O₄), or hydrazine (N₂H₄)). A chemically cleavable linker is non-enzymatically cleavable. In embodiments, the cleavable linker is cleaved by contacting the cleavable linker with a cleaving agent. In embodiments, the cleaving agent is a phosphine containing reagent (e.g., TCEP or THPP), sodium dithionite (Na₂S₂O₄), weak acid, hydrazine (N₂H₄), Pd(0), or light-irradiation (e.g., ultraviolet radiation). In embodiments, cleaving includes removing. A “cleavable site” or “scissile linkage” in the context of a polynucleotide is a site which allows controlled cleavage of the polynucleotide strand (e.g., the linker, the primer, or the polynucleotide) by chemical, enzymatic, or photochemical means known in the art and described herein. A scissile site may refer to the linkage of a nucleotide between two other nucleotides in a nucleotide strand (i.e., an internucleosidic linkage). In embodiments, the scissile linkage can be located at any position within the one or more nucleic acid molecules, including at or near a terminal end (e.g., the 3′ end of an oligonucleotide) or in an interior portion of the one or more nucleic acid molecules. In embodiments, conditions suitable for separating a scissile linkage include a modulating the pH and/or the temperature. In embodiments, a scissile site can include at least one acid-labile linkage. For example, an acid-labile linkage may include a phosphoramidate linkage. In embodiments, a phosphoramidate linkage can be hydrolysable under acidic conditions, including mild acidic conditions such as trifluoroacetic acid and a suitable temperature (e.g., 30° C.), or other conditions known in the art, for example Matthias Mag, et al Tetrahedron Letters, Volume 33, Issue 48, 1992, 7319-7322. In embodiments, the scissile site can include at least one photolabile internucleosidic linkage (e.g., o-nitrobenzyl linkages, as described in Walker et al, J. Am. Chem. Soc. 1988, 110, 21, 7170-7177), such as o-nitrobenzyloxymethyl or p-nitrobenzyloxymethyl group(s). In embodiments, the scissile site includes at least one uracil nucleobase. In embodiments, a uracil nucleobase can be cleaved with a uracil DNA glycosylase (UDG) or formamidopyrimidine DNA glycosylase (Fpg). In embodiments, the scissile linkage site includes a sequence-specific nicking site having a nucleotide sequence that is recognized and nicked by a nicking endonuclease enzyme or a uracil DNA glycosylase.

As used herein, the term “removable” group, e.g., a label or a blocking group or protecting group, is used in accordance with its plain and ordinary meaning and refers to a chemical group that can be removed from a nucleotide analogue such that a DNA polymerase can extend the nucleic acid (e.g., a primer or extension product) by the incorporation of at least one additional nucleotide. Removal may be by any suitable method, including enzymatic, chemical, or photolytic cleavage. Removal of a removable group, e.g., a blocking group, does not require that the entire removable group be removed, only that a sufficient portion of it be removed such that a DNA polymerase can extend a nucleic acid by incorporation of at least one additional nucleotide using a nucleotide or nucleotide analogue.

As used herein, the terms “blocking moiety,” “reversible blocking group,” “reversible terminator” and “reversible terminator moiety” are used in accordance with their plain and ordinary meanings and refer to a cleavable moiety which does not interfere with incorporation of a nucleotide including it by a polymerase (e.g., DNA polymerase, modified DNA polymerase), but prevents further strand extension until removed (“unblocked”). For example, a reversible terminator may refer to a blocking moiety located, for example, at the 3′ position of the nucleotide and may be a chemically cleavable moiety such as an allyl group, an azidomethyl group or a methoxymethyl group, or may be an enzymatically cleavable group such as a phosphate ester. Suitable nucleotide blocking moieties are described in applications WO 2004/018497, U.S. Pat. Nos. 7,057,026, 7,541,444, WO 96/07669, U.S. Pat. Nos. 5,763,594, 5,808,045, 5,872,244 and 6,232,465 the contents of which are incorporated herein by reference in their entirety. The nucleotides may be labelled or unlabeled. The nucleotides may be modified with reversible terminators useful in methods provided herein and may be 3′-O-blocked reversible or 3′-unblocked reversible terminators. In nucleotides with 3′-O-blocked reversible terminators, the blocking group may be represented as —OR [reversible terminating (capping) group], wherein 0 is the oxygen atom of the 3′-OH of the pentose and R is the blocking group, while the label is linked to the base, which acts as a reporter and can be cleaved. 3′-O-blocked reversible terminators are known in the art, and may be, for instance, a 3′-ONH₂ reversible terminator, a 3′-O-allyl reversible terminator, or a 3′-O-azidomethyl reversible terminator. In embodiments, the reversible terminator moiety is

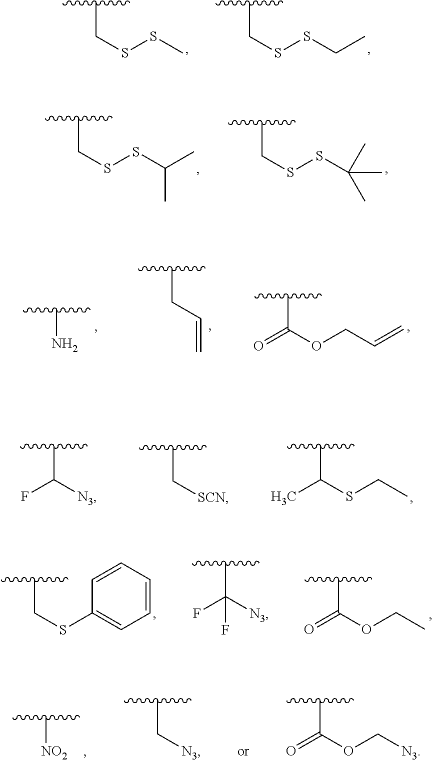

The term “allyl” as described herein refers to an unsubstituted methylene attached to a vinyl group (i.e., —CH═CH₂), having the formula

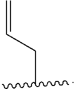 [SMILES: *CC=C]

In embodiments, the reversible terminator moiety is

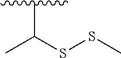 [SMILES: *C(C)SSC]

as described in U.S. Pat. No. 10,738,072, which is incorporated herein by reference for all purposes. For example, a nucleotide including a reversible terminator moiety may be represented by the formula:

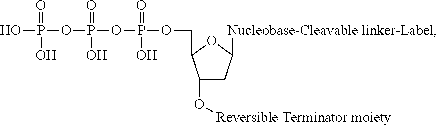

where the nucleobase is adenine or adenine analogue, thymine or thymine analogue, guanine or guanine analogue, or cytosine or cytosine analogue.

As used herein, the term “label” or “labels” is used in accordance with their plain and ordinary meanings and refer to molecules that can directly or indirectly produce or result in a detectable signal either by themselves or upon interaction with another molecule. Non-limiting examples of detectable labels include fluorescent dyes, biotin, digoxin, haptens, and epitopes. In general, a dye is a molecule, compound, or substance that can provide an optically detectable signal, such as a colorimetric, luminescent, bioluminescent, chemiluminescent, phosphorescent, or fluorescent signal. In embodiments, the label is a dye. In embodiments, the dye is a fluorescent dye. Non-limiting examples of dyes, some of which are commercially available, include CF dyes (Biotium, Inc.), Alexa Fluor dyes (Thermo Fisher), DyLight dyes (Thermo Fisher), Cy dyes (GE Healthscience), IRDyes (Li-Cor Biosciences, Inc.), and HiLyte dyes (Anaspec, Inc.). In embodiments, a particular nucleotide type is associated with a particular label, such that identifying the label identifies the nucleotide with which it is associated. In embodiments, the label is luciferin that reacts with luciferase to produce a detectable signal in response to one or more bases being incorporated into an elongated complementary strand, such as in pyrosequencing. In embodiment, a nucleotide includes a label (such as a dye). In embodiments, the label is not associated with any particular nucleotide, but detection of the label identifies whether one or more nucleotides having a known identity were added during an extension step (such as in the case of pyrosequencing).

In embodiments, the detectable label is a fluorescent dye. In embodiments, the detectable label is a fluorescent dye capable of exchanging energy with another fluorescent dye (e.g., fluorescence resonance energy transfer (FRET) chromophores).

In embodiments, the detectable moiety is a moiety of a derivative of one of the detectable moieties described immediately above, wherein the derivative differs from one of the detectable moieties immediately above by a modification resulting from the conjugation of the detectable moiety to a compound described herein.

The term “cyanine” or “cyanine moiety” as described herein refers to a detectable moiety containing two nitrogen groups separated by a polymethine chain. In embodiments, the cyanine moiety has 3 methine structures (i.e., cyanine 3 or Cy3). In embodiments, the cyanine moiety has 5 methine structures (i.e., cyanine 5 or Cy5). In embodiments, the cyanine moiety has 7 methine structures (i.e., cyanine 7 or Cy7).

As used herein, the term “DNA polymerase” and “nucleic acid polymerase” are used in accordance with their plain ordinary meanings and refer to enzymes capable of synthesizing nucleic acid molecules from nucleotides (e.g., deoxyribonucleotides). Typically, a DNA polymerase adds nucleotides to the 3′-end of a DNA strand, one nucleotide at a time. In embodiments, the DNA polymerase is a Pol I DNA polymerase, Pol II DNA polymerase, Pol III DNA polymerase, Pol IV DNA polymerase, Pol V DNA polymerase, Pol β DNA polymerase, Pol μ DNA polymerase, Pol λ DNA polymerase, Pol σ DNA polymerase, Pol α DNA polymerase, Pol δ DNA polymerase, Pol ε DNA polymerase, Pol η DNA polymerase, Pol ι DNA polymerase, Pol κ DNA polymerase, Pol ζ DNA polymerase, Pol γ DNA polymerase, Pol θ DNA polymerase, Pol ν DNA polymerase, or a thermophilic nucleic acid polymerase (e.g. Therminator γ, 9° N polymerase (exo-), Therminator II, Therminator III, or Therminator IX). In embodiments, the DNA polymerase is a modified archaeal DNA polymerase. In embodiments, the polymerase is a reverse transcriptase. In embodiments, the polymerase is a mutant P. abyssi polymerase (e.g., such as a mutant P. abyssi polymerase described in WO 2018/148723 or WO 2020/056044).

As used herein, the term “exonuclease activity” is used in accordance with its ordinary meaning in the art, and refers to the removal of a nucleotide from a nucleic acid by a DNA polymerase. For example, during polymerization, nucleotides are added to the 3′ end of the primer strand. Occasionally a DNA polymerase incorporates an incorrect nucleotide to the 3′-OH terminus of the primer strand, wherein the incorrect nucleotide cannot form a hydrogen bond to the corresponding base in the template strand. Such a nucleotide, added in error, is removed from the primer as a result of the 3′ to 5′ exonuclease activity of the DNA polymerase. In embodiments, exonuclease activity may be referred to as “proofreading.” When referring to 3′-5′ exonuclease activity, it is understood that the DNA polymerase facilitates a hydrolyzing reaction that breaks phosphodiester bonds at either the 3′ end of a polynucleotide chain to excise the nucleotide. In embodiments, 3′-5′ exonuclease activity refers to the successive removal of nucleotides in single-stranded DNA in a 3′ →5′ direction, releasing deoxyribonucleoside 5′-monophosphates one after another. Methods for quantifying exonuclease activity are known in the art, see for example Southworth et al, PNAS Vol 93, 8281-8285 (1996).

As used herein, the term “incorporating” or “chemically incorporating,” when used in reference to a primer and cognate nucleotide, refers to the process of joining the cognate nucleotide to the primer or extension product thereof by formation of a phosphodiester bond.

As used herein, the term “selective” or “selectivity” or the like of a compound refers to the compound's ability to discriminate between molecular targets. When used in the context of sequencing, such as in “selectively sequencing,” this term refers to sequencing one or more target polynucleotides from an original starting population of polynucleotides, and not sequencing non-target polynucleotides from the starting population. Typically, selectively sequencing one or more target polynucleotides involves differentially manipulating the target polynucleotides based on known sequence. For example, target polynucleotides may be hybridized to a probe oligonucleotide that may be labeled (such as with a member of a binding pair) or bound to a surface. In embodiments, hybridizing a target polynucleotide to a probe oligonucleotide includes the step of displacing one strand of a double-stranded nucleic acid. Probe-hybridized target polynucleotides may then be separated from non-hybridized polynucleotides, such as by removing probe-bound polynucleotides from the starting population or by washing away polynucleotides that are not bound to a probe. The result is a selected subset of the starting population of polynucleotides, which is then subjected to sequencing, thereby selectively sequencing the one or more target polynucleotides.

As used herein, the terms “specific”, “specifically”, “specificity”, or the like of a compound refers to the compound's ability to cause a particular action, such as binding, to a particular molecular target with minimal or no action to other proteins in the cell.

As used herein, the terms “bind” and “bound” are used in accordance with their plain and ordinary meanings and refer to an association between atoms or molecules. The association can be direct or indirect. For example, bound atoms or molecules may be directly bound to one another, e.g., by a covalent bond or non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). As a further example, two molecules may be bound indirectly to one another by way of direct binding to one or more intermediate molecules, thereby forming a complex.

As used herein, the terms “sequencing”, “sequence determination”, “determining a nucleotide sequence”, and the like include determination of partial as well as full sequence information, including the identification, ordering, or locations of the nucleotides that include the polynucleotide being sequenced, and inclusive of the physical processes for generating such sequence information. That is, the term includes sequence comparisons, fingerprinting, and like levels of information about a target polynucleotide, as well as the express identification and ordering of nucleotides in a target polynucleotide. The term also includes the determination of the identification, ordering, and locations of one, two, or three of the four types of nucleotides within a target polynucleotide. Sequencing methods, such as those outlined in U.S. Pat. No. 5,302,509 can be carried out using the nucleotides described herein. The sequencing methods are preferably carried out with the target polynucleotide arrayed on a solid substrate. Multiple target polynucleotides can be immobilized on the solid support through linker molecules, or can be attached to particles, e.g., microspheres, which can also be attached to a solid substrate. The solid substrate is in the form of a chip, a bead, a well, a capillary tube, a slide, a wafer, a filter, a fiber, a porous media, or a column. In embodiments, the solid substrate is gold, quartz, silica, plastic, glass, diamond, silver, metal, or polypropylene. In embodiments, the solid substrate is porous.

As used herein, the term “sequencing reaction mixture” is used in accordance with its plain and ordinary meaning and refers to an aqueous mixture that contains the reagents sufficient to allow a dNTP or dNTP analogue to add a nucleotide to a DNA strand by a DNA polymerase. In embodiments, the sequencing reaction mixture includes a buffer. In embodiments, the buffer includes an acetate buffer, 3-(N-morpholino) propanesulfonic acid (MOPS) buffer, N-(2-Acetamido)-2-aminoethanesulfonic acid (ACES) buffer, phosphate-buffered saline (PBS) buffer, 4-(2-hydroxyethyl)-1-piperazineethanesulfonic acid (HEPES) buffer, N-(1,1-Dimethyl-2-hydroxyethyl)-3-amino-2-hydroxypropanesulfonic acid (AMPSO) buffer, borate buffer (e.g., borate buffered saline, sodium borate buffer, boric acid buffer), 2-Amino-2-methyl-1,3-propanediol (AMPD) buffer, N-cyclohexyl-2-hydroxyl-3-aminopropanesulfonic acid (CAPSO) buffer, 2-Amino-2-methyl-1-propanol (AMP) buffer, 4-(Cyclohexylamino)-1-butanesulfonic acid (CABS) buffer, glycine-NaOH buffer, N-Cyclohexyl-2-aminoethanesulfonic acid (CHES) buffer, tris(hydroxymethyl)aminomethane (Tris) buffer, or a N-cyclohexyl-3-aminopropanesulfonic acid (CAPS) buffer. In embodiments, the buffer is a borate buffer. In embodiments, the buffer is a CHES buffer. In embodiments, the sequencing reaction mixture includes nucleotides, wherein the nucleotides include a reversible terminating moiety and a label covalently linked to the nucleotide via a cleavable linker. In embodiments, the sequencing reaction mixture includes a buffer, DNA polymerase, detergent (e.g., Triton X), a chelator (e.g., EDTA), or salts (e.g., ammonium sulfate, magnesium chloride, sodium chloride, or potassium chloride).

As used herein, the term “sequencing cycle” is used in accordance with its plain and ordinary meaning and refers to incorporating one or more nucleotides (e.g., nucleotide analogues) to the 3′ end of a polynucleotide with a polymerase, and detecting one or more labels that identify the one or more nucleotides incorporated. The sequencing may be accomplished by, for example, sequencing by synthesis, pyrosequencing, and the like. In embodiments, a sequencing cycle includes extending a complementary polynucleotide by incorporating a first nucleotide using a polymerase, wherein the polynucleotide is hybridized to a template nucleic acid, detecting the first nucleotide, and identifying the first nucleotide. In embodiments, to begin a sequencing cycle, one or more differently labeled nucleotides and a DNA polymerase can be introduced. Following nucleotide addition, signals produced (e.g., via excitation and emission of a detectable label) can be detected to determine the identity of the incorporated nucleotide (based on the labels on the nucleotides). Reagents can then be added to remove the 3′ reversible terminator and to remove labels from each incorporated base. Reagents, enzymes and other substances can be removed between steps by washing. Cycles may include repeating these steps, and the sequence of each cluster is read over the multiple repetitions.

“Hybridize” shall mean the annealing of one single-stranded nucleic acid sequence (such as a primer) to another nucleic acid sequence based on the well-understood principle of sequence complementarity. In an embodiment the other nucleic acid sequence is a single-stranded nucleic acid. The propensity for hybridization between nucleic acid sequences depends on the temperature and ionic strength of their milieu, the length of the nucleic acids and the degree of complementarity. The effect of these parameters on hybridization is described in, for example, Sambrook J., Fritsch E. F., Maniatis T., Molecular cloning: a laboratory manual, Cold Spring Harbor Laboratory Press, New York (1989). As used herein, hybridization of a primer, or of a DNA extension product, respectively, is extendable by creation of a phosphodiester bond with an available nucleotide or nucleotide analogue capable of forming a phosphodiester bond, therewith. For example, hybridization can be performed at a temperature ranging from 15° C. to 95° C. In some embodiments, the hybridization is performed at a temperature of about 20° C., about 25° C., about 30° C., about 35° C., about 40° C., about 45° C., about 50° C., about 55° C., about 60° C., about 65° C., about 70° C., about 75° C., about 80° C., about 85° C., about 90° C., or about 95° C. In other embodiments, the stringency of the hybridization can be further altered by the addition or removal of components of the buffered solution. In some embodiments, nucleic acids, or portions thereof, that are configured to hybridize are often about 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, 99% or more or 100% complementary to each other over a contiguous portion of nucleic acid sequence. A specific hybridization discriminates over non-specific hybridization interactions (e.g., two nucleic acids that a not configured to specifically hybridize, e.g., two nucleic acids that are 80% or less, 70% or less, 60% or less or 50% or less complementary) by about 2-fold or more, often about 10-fold or more, and sometimes about 100-fold or more, 1000-fold or more, 10,000-fold or more, 100,000-fold or more, or 1,000,000-fold or more. Two nucleic acid strands that are hybridized to each other can form a duplex which includes a double-stranded portion of nucleic acid.

As used herein, the term “extension” or “elongation” is used in accordance with their plain and ordinary meanings and refer to synthesis by a polymerase of a new polynucleotide strand complementary to a template strand by adding free nucleotides (e.g., dNTPs) from a reaction mixture that are complementary to the template in the 5′-to-3′ direction. Extension includes condensing the 5′-phosphate group of the dNTPs with the 3′-hydroxy group at the end of the nascent (elongating) DNA strand.

As used herein, the term “sequencing read” is used in accordance with its plain and ordinary meaning and refers to an inferred sequence of base pairs (or base pair probabilities) corresponding to all or part of a single DNA fragment. Sequencing technologies vary in the length of reads produced. A sequencing read may include 10, 20, 30, 40, 50, 60, 70, 80, 90, 100, 150, 200, 250, or more nucleotide bases. Reads of length 20-40 base pairs (bp) are referred to as ultra-short. Typical sequencers produce read lengths in the range of 100-500 bp. Read length is a factor which can affect the results of biological studies. For example, longer read lengths improve the resolution of de novo genome assembly and detection of structural variants. In some embodiments, a sequencing read may include 500, 600, 700, 800, 900, 1000, 1100, 1200, 1300, 1400, 1500, or more nucleotide bases.

As used herein, the term “k-mer” is used in accordance with its plain and ordinary meaning and refers to subsequences of a larger sequence string, wherein each k-mer is of length k. Algorithms for determining overlaps between sequence data may involve identification of k-mers between reads. Without being bound by theory, sequences that share a large number of k-mers are likely to come from the same region of the sequence to be identified, e.g., a genomic sequence. The value of k is the length of the matched region and is typically on the order of 10-30 base pairs. These regions can be found rapidly using data structures such as suffix trees or hash tables. For two overlapping reads to share a k-mer, the two reads will typically have either low error rates or be sufficiently long to compensate for a high chance of errors. However, for sequencing reads having relatively frequent errors, the method can be modified to allow errors in the k-mers. For example, previously developed algorithms have used spaced k-mers with “don't care” positions to allow for substitutions as well as to increase sensitivity over contiguous k-mers. Algorithms having such spaced k-mers are described in for example, Navarro, G. (2001) ACM Computing Surveys 33:31-88; and Farach-Colton, et al. (2007) J. Computer and Sys. Sci. 73:1035-1044, the disclosures of which are incorporated herein by reference in their entireties for all purposes.

As used herein, a “single cell” refers to one cell. Single cells useful in the methods described herein can be obtained from a tissue of interest, or from a biopsy, blood sample, or cell culture. Additionally, cells from specific organs, tissues, tumors, neoplasms, or the like can be obtained and used in the methods described herein. In general, cells from any population can be used in the methods, such as a population of prokaryotic or eukaryotic organisms, including bacteria or yeast.

The terms “cellular component” is used in accordance with its ordinary meaning in the art and refers to any organelle, nucleic acid, protein, or analyte that is found in a prokaryotic, eukaryotic, archaeal, or other organismic cell type. Examples of cellular components (e.g., a component of a cell) include RNA transcripts, proteins, membranes, lipids, and other analytes.

A “gene” refers to a polynucleotide sequence that is capable of conferring biological function after being transcribed and/or translated. Functionally, a genome is subdivided into genes. Each gene is a nucleic acid sequence that encodes an RNA or polypeptide. A gene is transcribed from DNA into RNA, which can either be non-coding (ncRNA) with a direct function, or an intermediate messenger (mRNA) that is then translated into protein. Typically a gene includes multiple sequence elements, such as for example, a coding element (i.e., a sequence that encodes a functional protein), non-coding element, and regulatory element. Each element may be as short as a few bp to 5 kb. In embodiments, the gene is the protein coding sequence of RNA. Non-limiting examples of genes include developmental genes (e.g., adhesion molecules, cyclin kinase inhibitors, Wnt family members, Pax family members, Winged helix family members, Hox family members, cytokines/lymphokines and their receptors, growth/differentiation factors and their receptors, neurotransmitters and their receptors); oncogenes (e.g., ABL1, BCL1, BCL2, BCL6, CBFA2, CBL, CSF1R, ERBA, ERBB, ERBB2, ETS1, ETS1, ETV6, FGR, FOS, FYN, HCR, HRAS, JUN, KRAS, LCK, LYN, MDM2, MLL, MYB, MYC, MYCL1, MYCN, NRAS, PIM1, PML, RET, SRC, TALL TCL3, and YES); tumor suppressor genes (e.g., APC, BRCA1, BRCA2, MADH4, MCC, NF1, NF2, RB1, TP53, and WT1); and enzymes (e.g., ACC synthases and oxidases, ACP desaturases and hydroxylases, ADP-glucose pyrophorylases, ATPases, alcohol dehydrogenases, amylases, amyloglucosidases, catalases, cellulases, chalcone synthases, chitinases, cyclooxygenases, decarboxylases, dextrinases, DNA and RNA polymerases, galactosidases, glucanases, glucose oxidases, granule-bound starch synthases, GTPases, helicases, hemicellulases, integrases, inulinases, invertases, isomerases, kinases, lactases, lipases, lipoxygenases, lysozymes, nopaline synthases, octopine synthases, pectinesterases, peroxidases, phosphatases, phospholipases, phosphorylases, phytases, plant growth regulator synthases, polygalacturonases, proteinases and peptidases, pullanases, recombinases, reverse transcriptases, RUBISCOs, topoisomerases, and xylanases). In embodiments, a gene includes at least one mutation associated with a disease or condition mediated by a mutant form of the gene.

Provided herein are methods and compositions for analyzing a sample (e.g., sequencing nucleic acids within a sample). A sample (e.g., a sample including nucleic acid) can be obtained from a suitable subject. A sample can be isolated or obtained directly from a subject or part thereof. In some embodiments, a sample is obtained indirectly from an individual or medical professional. A sample can be any specimen that is isolated or obtained from a subject or part thereof. A sample can be any specimen that is isolated or obtained from multiple subjects. Non-limiting examples of specimens include fluid or tissue from a subject, including, without limitation, blood or a blood product (e.g., serum, plasma, platelets, buffy coats, or the like), umbilical cord blood, chorionic villi, amniotic fluid, cerebrospinal fluid, spinal fluid, lavage fluid (e.g., lung, gastric, peritoneal, ductal, ear, arthroscopic), a biopsy sample, celocentesis sample, cells (blood cells, lymphocytes, placental cells, stem cells, bone marrow derived cells, embryo or fetal cells) or parts thereof (e.g., mitochondrial, nucleus, extracts, or the like), urine, feces, sputum, saliva, nasal mucous, prostate fluid, lavage, semen, lymphatic fluid, bile, tears, sweat, breast milk, breast fluid, the like or combinations thereof. A fluid or tissue sample from which nucleic acid is extracted may be acellular (e.g., cell-free). Non-limiting examples of tissues include organ tissues (e.g., liver, kidney, lung, thymus, adrenals, skin, bladder, reproductive organs, intestine, colon, spleen, brain, the like or parts thereof), epithelial tissue, hair, hair follicles, ducts, canals, bone, eye, nose, mouth, throat, ear, nails, the like, parts thereof or combinations thereof. A sample may include cells or tissues that are normal, healthy, diseased (e.g., infected), and/or cancerous (e.g., cancer cells). A sample obtained from a subject may include cells or cellular material (e.g., nucleic acids) of multiple organisms (e.g., virus nucleic acid, fetal nucleic acid, bacterial nucleic acid, parasite nucleic acid).

In some embodiments, a sample includes nucleic acid, or fragments thereof. A sample can include nucleic acids obtained from one or more subjects. In some embodiments a sample includes nucleic acid obtained from a single subject. In some embodiments, a sample includes a mixture of nucleic acids. A mixture of nucleic acids can include two or more nucleic acid species having different nucleotide sequences, different fragment lengths, different origins (e.g., genomic origins, cell or tissue origins, subject origins, the like or combinations thereof), or combinations thereof. A sample may include synthetic nucleic acid.

A subject can be any living or non-living organism, including but not limited to a human, non-human animal, plant, bacterium, fungus, virus or protist. A subject may be any age (e.g., an embryo, a fetus, infant, child, adult). A subject can be of any sex (e.g., male, female, or combination thereof). A subject may be pregnant. In some embodiments, a subject is a mammal. In some embodiments, a subject is a human subject. A subject can be a patient (e.g., a human patient). In some embodiments a subject is suspected of having a genetic variation or a disease or condition associated with a genetic variation.

As used herein, the term “consensus sequence” refers to a sequence that shows the nucleotide most commonly found at each position within the nucleic acid sequences of group of sequences (e.g., a group of sequencing reads) aligned at that position. A consensus sequence is often “assembled” from shorter sequence reads that are at least partially overlapping. Where two sequences contain overlapping sequence information aligned at one end and non-overlapping sequence information at opposite ends, the consensus sequence formed from the two sequences will be longer than either sequence individually. Aligning multiple such sequences allows for assembly of many short sequences into much longer consensus sequences representative of a longer sample polynucleotide. In embodiments, aligned sequences used to generate a consensus sequence may contain gaps (e.g., representative of nucleotides not appearing in a given read because they were extended during a dark cycle and not identified).

In some embodiments, a nucleic acid (e.g., an adapter, linear nucleic acid molecule, or a primer) includes a molecular identifier or a molecular barcode. As used herein, the term “molecular barcode” (which may be referred to as a “tag”, a “barcode”, a “molecular identifier”, an “identifier sequence” or a “unique molecular identifier” (UMI)) refers to any material (e.g., a nucleotide sequence, a nucleic acid molecule feature) that is capable of distinguishing an individual molecule in a large heterogeneous population of molecules. In embodiments, a barcode is unique in a pool of barcodes that differ from one another in sequence, or is uniquely associated with a particular sample polynucleotide in a pool of sample polynucleotides. In embodiments, every barcode in a pool of adapters is unique, such that sequencing reads including the barcode can be identified as originating from a single sample polynucleotide molecule on the basis of the barcode alone. In other embodiments, individual barcode sequences may be used more than once, but adapters including the duplicate barcodes are associated with different sequences and/or in different combinations of barcoded adaptors, such that sequence reads may still be uniquely distinguished as originating from a single sample polynucleotide molecule on the basis of a barcode and adjacent sequence information (e.g., sample polynucleotide sequence, and/or one or more adjacent barcodes). In embodiments, barcodes are about or at least about 5, 6, 7, 8, 9, 10, 15, 20, 25, 30, 40, 50, 75 or more nucleotides in length. In embodiments, barcodes are shorter than 20, 15, 10, 9, 8, 7, 6, or 5 nucleotides in length. In embodiments, barcodes are about 10 to about 50 nucleotides in length, such as about 15 to about 40 or about 20 to about 30 nucleotides in length. In a pool of different barcodes, barcodes may have the same or different lengths. In general, barcodes are of sufficient length and include sequences that are sufficiently different to allow the identification of sequencing reads that originate from the same sample polynucleotide molecule. In embodiments, each barcode in a plurality of barcodes differs from every other barcode in the plurality by at least three nucleotide positions, such as at least 3, 4, 5, 6, 7, 8, 9, 10, or more nucleotide positions. In some embodiments, substantially degenerate barcodes may be known as random. In some embodiments, a barcode may include a nucleic acid sequence from within a pool of known sequences. In some embodiments, the barcodes may be pre-defined.

In embodiments, a nucleic acid (e.g., an adapter, linear nucleic acid molecule, or primer) includes a sample barcode. In general, a “sample barcode” is a nucleotide sequence that is sufficiently different from other sample barcodes to allow the identification of the sample source based on sample barcode sequence(s) with which they are associated. In embodiments, a plurality of nucleotides (e.g., all nucleotides from a particular sample source, or sub-sample thereof) are joined to a first sample barcode, while a different plurality of nucleotides (e.g., all nucleotides from a different sample source, or different subsample) are joined to a second sample barcode, thereby associating each plurality of polynucleotides with a different sample barcode indicative of sample source. In embodiments, each sample barcode in a plurality of sample barcodes differs from every other sample barcode in the plurality by at least three nucleotide positions, such as at least 3, 4, 5, 6, 7, 8, 9, 10, or more nucleotide positions. In some embodiments, substantially degenerate sample barcodes may be known as random. In some embodiments, a sample barcode may include a nucleic acid sequence from within a pool of known sequences. In some embodiments, the sample barcodes may be pre-defined. In embodiments, the sample barcode includes about 1 to about 10 nucleotides. In embodiments, the sample barcode includes about 3, 4, 5, 6, 7, 8, 9, or about 10 nucleotides. In embodiments, the sample barcode includes about 3 nucleotides. In embodiments, the sample barcode includes about 5 nucleotides. In embodiments, the sample barcode includes about 7 nucleotides. In embodiments, the sample barcode includes about 10 nucleotides. In embodiments, the sample barcode includes about 6 to about 10 nucleotides.

Where a range of values is provided, it is understood that each intervening value, to the tenth of the unit of the lower limit unless the context clearly indicates otherwise, between the upper and lower limit of that range, and any other stated or unstated intervening value in, or smaller range of values within, that stated range is encompassed within the invention. The upper and lower limits of any such smaller range (within a more broadly recited range) may independently be included in the smaller ranges, or as particular values themselves, and are also encompassed within the invention, subject to any specifically excluded limit in the stated range. Where the stated range includes one or both of the limits, ranges excluding either or both of those included limits are also included in the invention.

The term “kit” is used in accordance with its plain ordinary meaning and refers to any delivery system for delivering materials or reagents for carrying out a method of the invention. Such delivery systems include systems that allow for the storage, transport, or delivery of reaction reagents (e.g., nucleotides, enzymes, nucleic acid templates, etc. in the appropriate containers) and/or supporting materials (e.g., buffers, written instructions for performing the reaction, etc.) from one location to another location. For example, kits include one or more enclosures (e.g., boxes) containing the relevant reaction reagents and/or supporting materials. Such contents may be delivered to the intended recipient together or separately. For example, a first container may contain an enzyme, while a second container contains nucleotides. In embodiments, the kit includes vessels containing one or more enzymes, primers, adaptors, or other reagents as described herein. Vessels may include any structure capable of supporting or containing a liquid or solid material and may include, tubes, vials, jars, containers, tips, etc. In embodiments, a wall of a vessel may permit the transmission of light through the wall. In embodiments, the vessel may be optically clear. The kit may include the enzyme and/or nucleotides in a buffer.

The methods and kits of the present disclosure may be applied, mutatis mutandis, to the sequencing of RNA, or to determining the identity of a ribonucleotide.

By aqueous solution herein is meant a liquid including at least 20 vol % water. In embodiments, aqueous solution includes at least 50%, for example at least 75 vol %, at least 95 vol %, above 98 vol %, or 100 vol % of water as the continuous phase.

The term “nucleic acid sequencing device” and the like means an integrated system of one or more chambers, ports, and channels that are interconnected and in fluid communication and designed for carrying out an analytical reaction or process, either alone or in cooperation with an appliance or instrument that provides support functions, such as sample introduction, fluid and/or reagent driving means, temperature control, detection systems, data collection and/or integration systems, for the purpose of determining the nucleic acid sequence of a template polynucleotide. Nucleic acid sequencing devices may further include valves, pumps, and specialized functional coatings on interior walls. Nucleic acid sequencing devices may include a receiving unit, or platen, that orients the flow cell such that a maximal surface area of the flow cell is available to be exposed to an optical lens. Other nucleic acid sequencing devices include those provided by Illumina™, Inc. (e.g., HiSeg™, MiSeg™, NextSeg™, or NovaSeg™ systems), Life Technologies™ (e.g., ABI PRISM™, or SOLiD™ systems), Pacific Biosciences (e.g., systems using SMRT™ Technology such as the Sequel™ or RS II™ systems), or Qiagen (e.g., Genereader™ system).

“Disease” or “condition” or “disease state” refers to any abnormal biological or aberrant condition of a cell, tissue, or organism. A disease may refer to a state of being or health status of a patient or subject. In some embodiments, the disease is a disease related to (e.g. caused by) an activated or overactive kinase or aberrant kinase activity. A disease state may be a consequence of, inter alia, an environmental pathogen, for example a viral infection (e.g., HIV/AIDS, hepatitis B, hepatitis C, influenza, measles, etc.), a bacterial infection, a parasitic infection, a fungal infection, or infection by some other organism. A disease state may also be the consequence of some other environmental agent, such as a chemical toxin or a chemical carcinogen. As used herein, a disease state further includes genetic disorders wherein one or more copies of a gene is altered or disrupted, thereby affecting its biological function. Exemplary genetic diseases include, but are not limited to polycystic kidney disease, familial multiple endocrine neoplasia type I, neurofibromatoses, Tay-Sachs disease, Huntington's disease, sickle cell anemia, thalassemia, and Down's syndrome, as well as others (see, e.g., The Metabolic and Molecular Bases of Inherited Diseases, 7th ed., McGraw-Hill Inc., New York). Other exemplary diseases include, but are not limited to, cancer, hypertension, Alzheimer's disease, neurodegenerative diseases, and neuropsychiatric disorders such as bipolar affective disorders or paranoid schizophrenic disorders. Disease states are monitored to determine the level or severity (e.g., the stage or progression) of one or more disease states of a subject and, more specifically, detect changes in the biological state of a subject which are correlated to one or more disease states (see, e.g., U.S. Pat. No. 6,218,122, which is incorporated by reference herein in its entirety). In embodiments, methods provided herein are also applicable to monitoring the disease state or states of a subject undergoing one or more therapies. Thus, the present disclosure also provides, in some embodiments, methods for determining or monitoring efficacy of a therapy or therapies (i.e., determining a level of therapeutic effect) upon a subject. In embodiments, methods of the present disclosure can be used to assess therapeutic efficacy in a clinical trial, e.g., as an early surrogate marker for success or failure in such a clinical trial. Within eukaryotic cells, there are hundreds to thousands of signaling pathways that are interconnected. For this reason, perturbations in the function of proteins within a cell have numerous effects on other proteins and the transcription of other genes that are connected by primary, secondary, and sometimes tertiary pathways. This extensive interconnection between the function of various proteins means that the alteration of any one protein is likely to result in compensatory changes in a wide number of other proteins. In particular, the partial disruption of even a single protein within a cell, such as by exposure to a drug or by a disease state which modulates the gene copy number (e.g., a genetic mutation), results in characteristic compensatory changes in the transcription of enough other genes that these changes in transcripts can be used to define a “signature” of particular transcript alterations which are related to the disruption of function, e.g., a particular disease state or therapy, even at a stage where changes in protein activity are undetectable.

As used herein, the term “neurodegenerative disease” refers to a disease or condition in which the function of a subject's nervous system becomes impaired. Examples of neurodegenerative diseases that may be detected method described herein include Alexander's disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, Cockayne syndrome, Corticobasal degeneration, Creutzfeldt-Jakob disease, frontotemporal dementia, Gerstmann-Straussler-Scheinker syndrome, Huntington's disease, HIV-associated dementia, Kennedy's disease, Krabbe's disease, kuru, Lewy body dementia, Machado-Joseph disease (Spinocerebellar ataxia type 3), Multiple sclerosis, Multiple System Atrophy, Narcolepsy, Neuroborreliosis, Parkinson's disease, Pelizaeus-Merzbacher Disease, Pick's disease, Primary lateral sclerosis, Prion diseases, Refsum's disease, Sandhoff s disease, Schilder's disease, Subacute combined degeneration of spinal cord secondary to Pernicious Anaemia, Schizophrenia, Spinocerebellar ataxia (multiple types with varying characteristics), Spinal muscular atrophy, Steele-Richardson-Olszewski disease, or Tabes dorsalis.

As used herein, the term “autoimmune disease” refers to a disease or condition in which a subject's immune system irregularly responds to one or more components (e.g. biomolecule, protein, cell, tissue, organ, etc.) of the subject. In some embodiments, an autoimmune disease is a condition in which the subject's immune system irregularly reacts to one or more components of the subject as if such components were not self. Exemplary autoimmune diseases that may be detected with a method provided herein include Acute Disseminated Encephalomyelitis (ADEM), Acute necrotizing hemorrhagic leukoencephalitis, Addison's disease, Agammaglobulinemia, Asthma, Allergic asthma, Allergic rhinitis, Alopecia areata, Amyloidosis, Ankylosing spondylitis, Anti-GBM/Anti-TBM nephritis, Antiphospholipid syndrome (APS), Arthritis, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune pancreatitis, Autoimmune retinopathy, Autoimmune thrombocytopenic purpura (ATP), Autoimmune thyroid disease, Axonal & neuronal neuropathies, Balo disease, Behcet's disease, Bullous pemphigoid, Cardiomyopathy, Castleman disease, Celiac sprue, Chagas disease, Chronic inflammatory demyelinating polyneuropathy (CIDP), Chronic recurrent multifocal osteomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Discoid lupus, Dressler's syndrome, Endometriosis, Eosinophilic fasciitis, Erythema nodosum, Experimental allergic encephalomyelitis, Evans syndrome, Fibrosing alveolitis, Giant cell arteritis (temporal arteritis), Glomerulonephritis, Goodpasture's syndrome, Graves' disease, Grave's ophthalmopathy, Guillain-Barre syndrome, Hashimoto's encephalitis, Hashimoto's thyroiditis, Hemolytic anemia, Henoch-Schonlein purpura, Herpes gestationis, Hypogammaglobulinemia, Ichthyosis, Idiopathic thrombocytopenic purpura (ITP), IgA nephropathy, IgG4-related sclerosing disease, Immunoregulatory lipoproteins, Inclusion body myositis, Inflammatory bowel disease, Insulin-dependent diabetes (type1), Interstitial cystitis, Juvenile arthritis, Juvenile diabetes, Kawasaki syndrome, Lambert-Eaton syndrome, Leukocytoclastic vasculitis, Lichen planus, Lichen sclerosus, Ligneous conjunctivitis, Linear IgA disease (LAD), Lupus (SLE), Lyme disease, chronic, Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neuromyelitis optica (Devic's), Neutropenia, Ocular cicatricial pemphigoid, Optic neuritis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatic, Polymyositis, Postmyocardial infarction syndrome, Postpericardiotomy syndrome, Progesterone dermatitis, Primary biliary cirrhosis, Primary sclerosing cholangitis, Psoriasis, Psoriatic, arthritis, Idiopathic pulmonary fibrosis, Pyoderma gangrenous, Pure red cell aplasia, Raynauds phenomenon, Reflex sympathetic dystrophy, Reiter's syndrome, Relapsing polychondritis, Restless legs syndrome, Retroperitoneal Fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopenic purpura (TTP), Tolosa-Hunt syndrome, Transverse myelitis, Ulcerative colitis, Undifferentiated connective tissue disease (UCTD), Uveitis, Vasculitis, Vesiculobullous dermatosis, Vitiligo, or Wegener's granulomatosis.

A primary immune deficiency disease (PIDDs) include rare, genetic disorders that impair the immune system. Without a functional immune response, people with PIDDs may be subject to chronic, debilitating infections, such as Epstein-Barr virus (EBV), which can increase the risk of developing cancer. Non-limiting examples of primary immunodeficiency diseases include Autoimmune Lymphoproliferative Syndrome (ALPS), APS-1 (APECED), BENTA Disease, Caspase Eight Deficiency State (CEDS), CARDS Deficiency and Other Syndromes of Susceptibility to Candidiasis, Chronic Granulomatous Disease (CGD), Common Variable Immunodeficiency (CVID), Congenital Neutropenia Syndromes, CTLA4 Deficiency, DOCK8 Deficiency, GATA2 Deficiency, Glycosylation Disorders with Immunodeficiency, Hyper-Immunoglobulin E Syndromes (HIES), Hyper-Immunoglobulin M Syndromes, Interferon Gamma, Interleukin 12 and Interleukin 23 Deficiencies, Leukocyte Adhesion Deficiency (LAD), LRBA Deficiency, PI3 Kinase Disease, PLCG2-associated Antibody Deficiency and Immune Dysregulation (PLAID), Severe Combined Immunodeficiency (SCID), STAT3 Dominant-Negative Disease, STAT3 Gain-of-Function Disease, Warts, Hypogammaglobulinemia, Infections, and Myelokathexis (WHIM) Syndrome, Wiskott-Aldrich Syndrome (WAS), X-Linked Agammaglobulinemia (XLA), X-Linked Lymphoproliferative Disease (XLP), and XMEN Disease.

As used herein, the term “cardiovascular disease” refers to a disease or condition affecting the heart or blood vessels. In embodiments, cardiovascular disease includes diseases caused by or exacerbated by atherosclerosis. Exemplary cardiovascular diseases that may be detected with a method provided herein include Alcoholic cardiomyopathy, Coronary artery disease, Congenital heart disease, Arrhythmogenic right ventricular cardiomyopathy, Restrictive cardiomyopathy, Noncompaction Cardiomyopathy, diabetes mellitus, hypertension, hyperhomocysteinemia, hypercholesterolemia, Atherosclerosis, Ischemic heart disease, Heart failure, Cor pulmonale, Hypertensive heart disease, Left ventricular hypertrophy, Coronary heart disease, (Congestive) heart failure, Hypertensive cardiomyopathy, Cardiac arrhythmias, Inflammatory heart disease, Endocarditis, Inflammatory cardiomegaly, Myocarditis, Valvular heart disease, stroke, or myocardial infarction. In embodiments, the disease is a cardiovascular disease associated with a gene fusion. Genome-wide association (GWA) studies revealed numerous potentially disease modifying genetic fusion events; see for example, Paone et al Front. Cardiovasc. Med., 1 Jun. 2018, which is incorporated herein by reference.

As used herein, the term “cancer” refers to all types of cancer, neoplasm or malignant tumors found in mammals, including leukemia, carcinomas and sarcomas. Exemplary cancers that may be detected with a method provided herein include cancer of the thyroid, endocrine system, brain, breast, cervix, colon, head & neck, liver, kidney, lung, non-small cell lung, melanoma, mesothelioma, ovary, pancreas, sarcoma, stomach, uterus or Medulloblastoma. Additional examples include, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be detected with a method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

The term “sarcoma” generally refers to a tumor which is made up of a substance like the embryonic connective tissue and is generally composed of closely packed cells embedded in a fibrillar or homogeneous substance. Sarcomas that may be detected with a method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be detected with a method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be detected with a method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

The term “aberrant” as used herein refers to different from normal. When used to described enzymatic activity, aberrant refers to activity that is greater or less than a normal control or the average of normal non-diseased control samples. Aberrant activity may refer to an amount of activity that results in a disease, wherein returning the aberrant activity to a normal or non-disease-associated amount (e.g. by administering a compound), results in reduction of the disease or one or more disease symptoms.

A “blocking element” refers to an agent (e.g., polynucleotide, protein, nucleotide) that reduces and/or inhibits nucleotide incorporation (i.e., extension of a primer) relative to the absence of the blocking element. In embodiments, the blocking element is a non-extendable oligomer (e.g., a 3′-blocked oligo). A blocking element on a nucleotide can be reversible, whereby the blocking moiety can be removed or modified to allow the 3′ hydroxyl to form a covalent bond with the 5′ phosphate of another nucleotide. For example, a reversible terminator may refer to a blocking moiety located, for example, at the 3′ position of the nucleotide and may be a chemically cleavable moiety such as an allyl group, an azidomethyl group or a methoxymethyl group. In embodiments the blocking moiety is not reversible (e.g., the blocking element including a blocking moiety irreversibly prevents extension). In embodiments, the blocking element includes an oligo having a 3′ dideoxynucleotide or similar modification to prevent extension by a polymerase and is used in conjunction with a non-strand displacing polymerase. In another example implementation, the blocking element includes one or more modified nucleotides including a cleavable linker (e.g., linked to the 5′, 3′, or the nucleobase) containing PEG, thereby blocking the extension. In another example implementation, the blocking element includes one or more modified nucleotides linked to biotin, to which a protein (e.g., streptavidin) can be bound, thereby blocking polymerase extension. In another example implementation, the blocking element includes a modified nucleotide, such as iso dGTP or iso dCTP, which are complementary to each other. In a reaction of polymerization lacking the appropriate complementary modified nucleotides, the extension of a primer is halted. In another example implementation, the blocking element includes one or more sequences which is recognized and bound by one or more single-stranded DNA-binding proteins, thereby blocking polymerase extension at the bound site. In another example implementation, the blocking element includes one or more sequences which are recognized and bound by one or more short RNA or PNA oligos, thereby blocking the extension by a DNA polymerase that cannot strand displace RNA or PNA.

The term “clonotype” is used in accordance with its ordinary meaning in the art and refers to a recombined nucleic acid which encodes an immune receptor or a portion thereof. For example, a clonotype refers to a recombined nucleic acid, usually extracted from a T cell or B cell, but which may also be from a cell-free source, which encodes a T cell receptor (TCR) or B cell receptor (BCR), or a portion thereof. In embodiments, clonotypes may encode all or a portion of a VDJ rearrangement of IgH, a DJ rearrangement of IgH, a VJ rearrangement of IgK, a VJ rearrangement of IgL, a VDJ rearrangement of TCR β, a DJ rearrangement of TCR β, a VJ rearrangement of TCR α, a VJ rearrangement of TCRγ, a VDJ rearrangement of TCR δ, a VD rearrangement of TCR δ, a Kde-V rearrangement, or the like. Clonotypes may also encode translocation breakpoint regions involving immune receptor genes, such as Bcl1-JH or Bcl2-JH. In one aspect, clonotypes have sequences that are sufficiently long to represent or reflect the diversity of the immune molecules that they are derived from consequently, clonotypes may vary widely in length. In some embodiments, clonotypes have lengths in the range of from 25 to 400 nucleotides; in other embodiments, clonotypes have lengths in the range of from 25 to 200 nucleotides.

A “immune repertoire” refers to the collection of T cell receptors and B cell receptors (e.g., immunoglobulin) that constitutes an organism's adaptive immune system.

A “convergence frequency” refers to the aggregate frequency of clones sharing a variable gene (excluding allele information).

A “convergent TCR group” is a set of T cell receptors (TCRs) that are similar in amino acid sequence and functionally equivalent, or are identical or assumed to be identical in amino acid sequence. It is generally assumed, owing to the amino acid similarity, that a convergent TCR group recognizes the same antigen. In some embodiments, convergent TCR group members are identical or assumed to be identical in the variable gene and CDR3 amino acid sequence despite having a different nucleotide sequence. Convergent TCR group members may result from differences in non-templated nucleotide bases at the VDJ junction that arise during the generation of a productive TCR gene rearrangement. To evaluate TCR convergence, for example, instances where TCRβ chains are identical in amino acid sequence but have distinct nucleotide sequences are determined.

The term “novel” as used herein is used in accordance with its ordinary meaning and refers to something new or having no precedent. Many gene fusions exist from two known genes, for example the BCR-ABL1 gene fusion is the result of a fusion of the ABL1 gene of chromosome 9 and the breakpoint cluster region BCR gene. The BCR-ABL1 fusion gene encodes for an active tyrosine kinase with transforming capacity, is prevalent in about 5% of pediatric acute lymphoblastic leukemia (ALL) cases and it is associated with poor prognosis. In cancerous cells, genetic rearrangements are more prevalent, and the probability one known gene (e.g., ABL1) may fuse with other previously unknown partner genes is increased. Thus, a novel fusion gene refers to a fusion gene wherein one gene, or portion thereof, is attached to a second gene, or portion thereof, wherein the novel fusion gene was not a priori known prior to examination.

The term “limit of detection” is used in accordance with its ordinary meaning and refers to the lowest quantity or concentration of a component that can be reliably detected (i.e., to a sufficient statistical significance).

A “locus” is used in accordance with its ordinary meaning and refers to a location of a gene or other DNA sequence on a chromosome. The Immunoglobulin Heavy (IGH) locus refers to a collection of located on chromosome 14 and is responsible for the production of heavy chain immunoglobulins, composed of several sub-loci, including V, D, J, C and S regions, which are involved in the process of antibody diversity. The IGH locus is responsible for the production of IgM, IgD, IgG, IgE, and IgA. The Immunoglobulin Kappa (IGK) locus refers to a collection of genes located on chromosome 2 and is responsible for the production of kappa light chain immunoglobulins, composed of V, J, and C regions, which are involved in the process of antibody diversity. The IGK locus is responsible for the production of IgM, IgD, IgG, IgE, and IgA. The Immunoglobulin Lambda (IGL) locus refers to a collection of genes located on chromosome 22 and is responsible for the production of lambda light chain immunoglobulins, composed of V, J, and C regions, which are involved in the process of antibody diversity.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### II. Methods

In an aspect is provided a method for detecting a genetic aberration in a sample from a subject, the method including: a) circularizing a plurality of linear nucleic acid molecules of the sample to form a plurality of circular template polynucleotides, wherein one or more of the linear nucleic acid molecules includes the genetic aberration, thereby forming one or more aberrant circular template polynucleotides; b) hybridizing a first primer and a second primer to the one or more aberrant circular template polynucleotides and extending with a polymerase to generate aberrant polynucleotide amplification products; and c) detecting the aberrant polynucleotide amplification products, wherein detecting includes hybridizing one or more sequencing primers to the aberrant polynucleotide amplification products and sequencing the aberrant polynucleotide amplification products. In embodiments, the genetic aberration is a single nucleotide variation (SNV), copy number variation (CNV), insertion or deletion (indel), internal tandem duplication, or a gene fusion (i.e., a fusion gene). In embodiments, the genetic feature is a clonotype. In embodiments, the genetic feature is a polynucleotide fusion (e.g., a fusion gene).

In an aspect is provided a method for detecting a fusion gene in a sample from a subject, the method including: a) circularizing a plurality of linear nucleic acid molecules of the sample to form a plurality of circular template polynucleotides, wherein one or more of the linear nucleic acid molecules include a fusion gene, thereby forming one or more fusion gene circular template polynucleotides; b) hybridizing a first primer and a second primer to the one or more fusion circular template polynucleotides and extending with a polymerase to generate fusion polynucleotide amplification products; and c) detecting the fusion polynucleotide amplification products, wherein detecting includes hybridizing one or more sequencing primers to the fusion polynucleotide amplification products and sequencing the fusion polynucleotide amplification products. In embodiments, the fusion gene is associated with cancer.

In another aspect is provided a method for detecting a fusion gene in a sample from a subject, the method including: circularizing a fusion linear nucleic acid molecule of the sample to form a fusion gene circular template polynucleotide, wherein the fusion linear nucleic acid molecule includes a fusion gene; circularizing a non-fusion linear nucleic acid molecule of the sample to form a non-fusion circular template polynucleotide, wherein the non-fusion linear nucleic acid molecule does not include the fusion gene; binding a blocking element to the non-fusion circular template polynucleotide; hybridizing a first primer to the fusion gene circular template polynucleotide and extending the first primer with a polymerase thereby generating a first fusion extension product; hybridizing a second primer to the first extension product and extending the second primer with a polymerase thereby generating a second extension product; sequencing the second extension product, or a complement thereof, thereby detecting the fusion gene.

In embodiments, detecting includes generating one or more sequencing reads via a next-generation sequencing method (e.g., sequencing by synthesis, sequencing-by-binding, sequencing by hybridization, sequencing by ligation, or pyrosequencing). In embodiments, detecting includes quantifying the number (i.e., counting) of amplification products (e.g., the number of fusion polynucleotide amplification products and/or non-fusion polynucleotide amplification products). In embodiments, the method further includes counting the number of unique amplification products containing the fusion gene. In embodiments, the method further includes counting the number of unique amplification products containing the fusion gene, counting the total number of amplification products containing a fusion gene, and calculating an amplification factor by dividing the number of unique amplification products containing fusion gene by the total number of amplification products containing a fusion gene. In embodiments, the method further includes dividing the total number of amplification products containing a fusion gene by the amplification factor and/or dividing the number of unique amplification products containing a fusion gene by the amplification factor. In embodiments, sequencing includes sequencing by synthesis, sequencing by binding, sequencing by ligation, or pyrosequencing. In embodiments, sequencing includes hybridizing a sequencing primer to said second extension product, or a complement thereof (a) extending the sequencing primer by incorporating a labeled nucleotide or labeled nucleotide analogue and (b) detecting the label to generate a signal for each incorporated nucleotide or nucleotide analogue.

In an aspect is provided a method for detecting cancer in a sample, the method including: circularizing a plurality of linear nucleic acid molecules of the sample to form a plurality of circular template polynucleotides, wherein one or more of the linear nucleic acid molecules include a fusion gene thereby forming one or more fusion gene circular template polynucleotides; and wherein one or more of the linear nucleic acid molecules do not include the fusion gene thereby forming one or more non-fusion gene circular template polynucleotides; hybridizing a first primer and a second primer to the one or more non-fusion circular template polynucleotides and the one or more fusion circular template polynucleotides and extending with a polymerase to generate a first number of non-fusion polynucleotide amplification products and a second number of fusion polynucleotide amplification products; detecting the fusion polynucleotide amplification products and detecting cancer in the sample when one or more of the fusion polynucleotide amplification products include a fusion gene associated with cancer.

In embodiments, generating amplification products of one or more non-fusion circular template polynucleotides includes hybridizing a third primer and a fourth primer to one or more non-fusion circular template polynucleotides and extending each primer with a polymerase. In embodiments, generating amplification products of one or more fusion circular template polynucleotides includes hybridizing a third primer and a fourth primer to one or more fusion circular template polynucleotides and extending each primer with a polymerase.

In embodiments, generating amplification products of one or more fusion and non-fusion circular template polynucleotides includes generating a third number of non-fusion polynucleotide amplification products and a fourth number of fusion polynucleotide amplification products, wherein the third number and the fourth number are substantially the same.

In embodiments, the third primer hybridizes upstream of a target sequence, and the fourth primer hybridizes downstream of a target sequence, wherein said target sequence includes a single-nucleotide variant, an insertion, a deletion, an internal tandem duplication, or a copy number variant.

In embodiments, the method includes amplifying the first extension product and/or the second extension product prior to sequencing, wherein amplifying comprises thermal bridge polymerase chain reaction (t-bPCR) amplification, chemical bridge polymerase chain reaction (c-bPCR) amplification, isothermal bridge amplification, chemical-thermal bridge polymerase chain reaction (cT-bPCR) amplification), rolling circle amplification (RCA), exponential rolling circle amplification (eRCA), recombinase polymerase amplification (RPA), or helicase dependent amplification (HDA).

In another aspect is provided a method of amplifying a plurality of polynucleotides, the method including, circularizing a plurality of linear nucleic acid molecules to form a plurality of circular template polynucleotides, wherein one or more of the linear nucleic acid molecules include a target sequence (e.g., a sequence of interest, such as a gene, SNV, CNV, indel, or a fusion gene); optionally binding a blocking element to one or more circular template polynucleotides that do not contain the target sequence; and hybridizing a first primer and a second primer to the circular template polynucleotides and extending with a polymerase amplification products, wherein the amount of amplification products including the target sequence are greater than the amount of amplification products that do not include the target sequence. In embodiments, the target sequence includes cancer somatic mutations, copy number variations, and gene fusions, including those involving novel partners or breakpoints.

In an aspect is provided a method of identifying the convergence frequency of a subject's immune repertoire (e.g., for predicting the clinical response of a subject to a therapy by identifying the convergence frequency of the subject's immune repertoire prior to receiving the therapy). In embodiments, the method further includes: a) obtaining from the subject a sample including one or more linear nucleic acid molecules including immune receptor sequences (e.g., T cell receptor (TCR), B cell receptor (BCR or Ab) targets); b) circularizing one or more linear nucleic acid molecules to form circular template polynucleotides including a continuous strand lacking free 5′ and 3′ ends and amplifying one or more circular template polynucleotides to generate a plurality of amplification products including the immune receptor sequences; c) sequencing the plurality of amplification products to generate a plurality of sequencing reads; d) identifying immune receptor clones by analyzing the plurality of sequencing reads; and e) detecting convergent immune receptor clones among the immune receptor clones, wherein the convergent immune receptor clones have a similar or identical amino acid sequence and a different nucleotide sequence. In embodiments, the method includes hybridizing a blocking element to the one or more circular template polynucleotides prior to amplifying. In embodiments, the method does not include hybridizing a blocking element to the one or more circular template polynucleotides. In embodiments, the method further includes determining the frequency of convergent immune receptor clones in the sample. In embodiments, the method further includes treating the subject with an immunotherapy when the frequency of convergent immune receptor clones in the sample is greater than a convergent frequency cutoff wherein sequences identifying the convergent immune receptor clones include CDR3 sequences.

As used herein, the term “immune repertoire” refers to the collection of T cell receptors and B cell receptors (e.g., immunoglobulin) that constitutes an organism's adaptive immune system. As used herein, the “convergence frequency” refers to the aggregate frequency of clones sharing a variable gene (excluding allele information).

In embodiments, the amplifying includes a multiplex amplification reaction including a plurality of amplification primer pairs including a plurality of j oining (J) gene primers directed to a majority of J genes of the target immune receptor (i.e., the primer pairs include complementary sequences to the J genes. The methods described herein permit targeting the joining genes with outward facing primers and thereby detect the V(D)J region, as opposed to directly target each V gene. In embodiments, the convergent immune receptor clones are identified using V gene identity and sequences including CDR3 amino acid sequences. In embodiment, the sequences identifying the convergent immune receptor clones include CDR1 and CDR3 sequences or CDR2 and CDR3 sequences. In embodiments, the convergent immune receptor clones have identical CDR3 amino acid sequences. In embodiments, the target immune receptor nucleic acid molecules include the FR1, CDR1, FR2, CDR2, FR3, and CDR3 coding regions of the target immune receptor.

As used herein, a “convergent TCR group” is a set of T cell receptors (TCRs) that are similar in amino acid sequence and functionally equivalent, or are identical or assumed to be identical in amino acid sequence. It is generally assumed, owing to the amino acid similarity, that a convergent TCR group recognizes the same antigen. In some embodiments, convergent TCR group members are identical or assumed to be identical in the variable gene and CDR3 amino acid sequence despite having a different nucleotide sequence. Convergent TCR group members may result from differences in non-templated nucleotide bases at the VDJ junction that arise during the generation of a productive TCR gene rearrangement. To evaluate TCR convergence, for example, instances where TCRβ chains are identical in amino acid sequence but have distinct nucleotide sequences are determined.

In some embodiments, the subject is treated with a therapy in a manner dependent on the frequency of the convergent immune receptor clones. For example, in some embodiments, a subject having a convergent immune receptor clone frequency greater than a convergent frequency cutoff indicates that the subject is candidate for the therapy whereas a subject having a convergent immune receptor clone frequency less than a convergent frequency cutoff indicates that the subject is not candidate for the therapy. In some embodiments, provided methods include identifying convergent immune receptor clones from the immune receptor clones present in the sample at a frequency of greater than 1 in 50,000. In some embodiments, the convergent frequency cutoff is a frequency of greater than 0.01. In some embodiments, the subject has cancer and is a candidate for an immunotherapy. In other embodiments, the subject is a candidate for a vaccination against an infectious agent or disease. In other embodiments, the subject is a candidate for autoimmune suppressant treatment.

In some embodiments, provided methods include identifying convergent immune receptor clones using V gene identity and sequences including CDR3 amino acid sequences. In some embodiments, provided methods include identifying convergent immune receptor clone using sequences that include CDR3 sequences, CDR1 and CDR3 sequences, or CDR2 and CDR3 sequences.

In some embodiments, provided methods include identifying convergent TCR clones as those including TCR variable and CDR3 rearrangements that are similar or identical in amino acid sequence but different in nucleotide sequence. For example, a significant fraction of the TCRs that differ from one another by one amino acid residue may nonetheless have similar or identical specificity for an antigen and so such TCRs may be considered convergent.

In some embodiments, a change in convergent TCR clone frequency over the course of a therapy treatment may be used as a predictor of response to the therapy. In a manner dependent on disease type and treatment, in some embodiments, responders may be distinguished from non-responders by an increase in the frequency of convergent TCR clones over the course of a therapy. For example, in cancers (or chronic viral infections) in which convergent TCR clones of the T cell population primarily consist of effector T cells of a progenitor exhausted T cell phenotype, a terminally exhausted phenotype or an effector phenotype among other T cell phenotypes, an increase in the frequency of convergent TCR clones over the course of a treatment may be indicative of an increase in the activity of anti-cancer (or anti-viral) T cells. In other cancers, convergent TCR clones may primarily be of T regulatory phenotype and an increase in the frequency of convergent TCR clones over the course of a therapy may indicate a poor prognosis.

In some embodiments, the measurement or determination of the frequency of convergent TCR clones is combined with other T cell repertoire features, such as for example, measurements of T cell clonal expansion, to improve the prediction of clinical responsiveness. In some embodiments, the measurement or determination of the frequency of convergent TCR clones is combined with B cell repertoire features, such as for example, measurements of B cell clonal expansion, to improve the prediction of clinical responsiveness. In some embodiments, the measurement or determination of the frequency of convergent TCR clones is combined with measurement or detection of expression of one or more genes relevant to immune response to improve the prediction of clinical responsiveness. Such immune response relevant genes include without limitation PD-1 and/or PD-L1 genes, interferon gamma pathway genes, and myeloid derived suppressor cell related genes. Procedures and reagents for detecting or measuring such gene expression are known in the art and include without limitation quantitative or semi-quantitative PCR analysis, comparative hybridization methods, or sequencing procedures and reagents and kits for use in same including without limitation TaqMan™ assays and the Oncomine™ Immune Response Research Assay (Thermo Fisher Scientific).

In embodiments, the method further includes identifying the clonotype. In embodiments, the method further includes quantifying the clonotypes present in a sample (e.g., rendering a clonotype profile). A “clonotype profile” refers to a collection of distinct clonotypes and their relative abundances derived from a population of lymphocytes, where, for example, relative abundance may be expressed as a frequency in a given population (i.e., a value between 0 and 1). Typically, the population of lymphocytes are obtained from a tissue sample. The term “clonotype profile” is related to, but more general than, the immunology concept of immune “repertoire” as described in Arstila et al., Science, 280: 958-961 (1999); and Kedzierska et al., Mol. Immunol., 45(3): 607-618 (2008).

In embodiments, clonotype profiles include at least 103 distinct clonotypes. In embodiments, clonotype profiles include at least 108 distinct clonotypes. In embodiments, clonotype profiles include at least 105 distinct clonotypes. In embodiments, clonotype profiles include at least 106 distinct clonotypes. In embodiments, such clonotype profiles may further include abundances (i.e., a quantification) or relative frequencies of each of the distinct clonotypes. In embodiments, a clonotype profile is a set of distinct recombined nucleotide sequences (with their abundances) that encode T receptors (TCRs) or B cell receptors (BCRs), or fragments thereof, respectively, in a population of lymphocytes of an individual, wherein the nucleotide sequences of the set have a correspondence (e.g., a 1:1 correspondence) with distinct lymphocytes or their clonal sub populations for substantially all of the lymphocytes of the population.

In embodiments, the fusion gene is associated with cancer when the fusion gene occurs at a relative frequency of at least 15% of the total number of fusion polynucleotide amplification products. In embodiments, the fusion gene is associated with cancer when the fusion gene occurs at a relative frequency of at least 20% of the total number of fusion polynucleotide amplification products. In embodiments, the fusion gene is associated with cancer when the fusion gene occurs at a relative frequency of at least 10% of the total number of fusion polynucleotide amplification products. In embodiments, the fusion gene is associated with cancer when the fusion gene occurs at a relative frequency of at least 5% of the total number of fusion polynucleotide amplification products. In embodiments, the fusion gene is associated with cancer when the fusion gene occurs at a relative frequency of at least 1% of the total number of fusion polynucleotide amplification products.

In embodiments, the fusion gene is associated with cancer when the fusion gene occurs at a relative frequency of at least 10⁻⁵ of the total number of fusion polynucleotide amplification products. In embodiments, the fusion gene is associated with cancer when the fusion gene occurs at a relative frequency of at least 10⁻⁴ of the total number of fusion polynucleotide amplification products. In embodiments, the fusion gene is associated with cancer when the fusion gene occurs at a relative frequency of at least 10⁻³ of the total number of fusion polynucleotide amplification products. In embodiments, the fusion gene is associated with cancer when the fusion gene occurs at a relative frequency of at least 10⁻⁶ of the total number of fusion polynucleotide amplification products.

In embodiments, the fusion gene is associated with cancer at a time prior to circularizing a plurality of linear nucleic acid molecules. For example, a subject has already been identified as having characteristic rearrangements and/or fusion genes associated with the malignancy from the diagnostic sample. In this case, the subject then wishes to determine whether they are still present and at what frequency (e.g., for post-treatment monitoring, where the malignancy is typically present at a low frequency; typical minimal residual disease (MRD) testing is performed to a limit of 10⁻⁴ or 10⁻⁵, though 10⁻⁶ is also possible). In embodiments, a plurality of samples is obtained at two or more time points, wherein each time point is at a fixed interval at least 7 days apart, at least 14 days apart, at least 30 days apart, at least 60 days apart, or at least 90 days apart. In embodiments, each time point is at least 1 month, at least 2 months, at least 3 months, at least 6 months, at least 9 months, at least 1 year, at least 2 years, or at least 3 years apart. In embodiments, each time point is about 1 month, about 2 months, about 3 months, about 6 months, about 9 months, about 1 year, about 2 years, or about 3 years apart. In embodiments, a first sample is obtained at a first time and a fusion gene (e.g., a novel fusion gene) is detected; and a second sample is obtained at a second, different, time and the fusion gene (e.g., the same previously novel fusion gene) is detected. In embodiments, the second time is after said subject received treatment.

In embodiments, the sample includes between about 20 ng to about 20 μg of linear nucleic acid molecules. In embodiments, the sample includes about 20 ng, about 30 ng, about 40 ng, about 50 ng, about 60 ng, about 80 ng, about 100 ng, about 200 ng, about 300 ng, about 400 ng, about 500 ng, about 1 μg, about 2 μg, about 3 μg, about 4 μg, about 5 μg, about 10 μs, about 15 μg, about 20 μg, or more of linear nucleic acid molecules. In embodiments, the sample includes at least 20 ng, at least 30 ng, at least 40 ng, at least 50 ng, at least 60 ng, at least 80 ng, at least 100 ng, at least 200 ng, at least 300 ng, at least 400 ng, at least 500 ng, at least 1 μg, at least 2 μg, at least 3 μg, at least 4 μg, at least 5 μg, at least 10 μg, at least 15 μg, at least 20 μg, or more of linear nucleic acid molecules.

In embodiments, circularizing includes contacting the plurality of linear nucleic acid molecules with a ligase capable of template-independent, intra-molecular ligation of linear nucleic acid molecules. In embodiments, circularizing includes contacting the plurality of linear nucleic acid molecules with a ligase capable of intra-molecular ligation of linear 500 bp or less nucleic acid molecules. In embodiments, the ligase is capable of intra-molecular ligation of linear 400 bp or less nucleic acid molecules. In embodiments, the ligase is capable of intra-molecular ligation of linear 300 bp or less nucleic acid molecules. In embodiments, the ligase is capable of intra-molecular ligation of linear 200 bp or less nucleic acid molecules. In embodiments, the ligase is capable of intra-molecular ligation of linear 100 bp or less nucleic acid molecules.

In embodiments, the ligase is a pre-adenylated ligase. In embodiments, the ligase a TS2126 RNA ligase. A TS2126 RNA ligase (commercially available under the trademarks THERMOPHAGE™ RNA ligase II or THERMOPHAGE™ ssDNA ligase or CircLigase™ ssDNA ligase (Epicenter Biotechnologies, Wisconsin, USA). CircLigase I™ has a low degree (about 30%) of adenylation, whereas CircLigase II™ includes a substantially adenylated form of TS2126 RNA ligase.

In embodiments, detecting the fusion polynucleotide amplification products includes contacting the fusion polynucleotide amplification products with one or more detection agent(s) and detecting the detection agent(s).

In embodiments, detecting the fusion polynucleotide amplification products includes contacting the fusion polynucleotide amplification products with one or more labeled-probe(s) and detecting the labeled oligonucleotide, wherein the labeled-probe hybridizes to a sequence in the fusion gene.

In embodiments, detecting the fusion polynucleotide amplification products includes hybridizing one or more sequencing primers to the fusion polynucleotide amplification products and sequencing the amplification products. In embodiments, sequencing includes (a) extending a sequencing primer by incorporating a labeled nucleotide or labeled nucleotide analogue and (b) detecting the label to generate a signal for each incorporated nucleotide or nucleotide analogue. In embodiments, sequencing the amplification products produces sequencing reads.

In embodiments, detecting further includes comparing the number of fusion gene amplification products and the number of non-fusion polynucleotide amplification products. In embodiments, one or more of the linear nucleic acid molecules do not include the fusion gene and circularizing forms one or more non-fusion circular template polynucleotides. In embodiments, the method further includes hybridizing a third primer and a fourth primer to the one or more non-fusion circular template polynucleotides and extending with a polymerase to generate non-fusion polynucleotide amplification products. In embodiments, detecting further includes comparing the quantity of fusion gene amplification products and the quantity of non-fusion polynucleotide amplification products.

In embodiments, binding the blocking element comprises binding the blocking element upstream of the first primer, wherein the blocking element binds about 1 to 150 nucleotides upstream relative to the first primer. In embodiments, the blocking element is an oligonucleotide.

In embodiments, the method includes binding a blocking element to the one or more non-fusion circular template polynucleotides. In embodiments, binding the blocking element includes binding the blocking element upstream of the first primer. In embodiments, the blocking element binds about 1 to 150 nucleotides upstream relative to the first primer. In embodiments, the blocking element includes an oligo, a protein, or a combination thereof. In embodiments, the blocking element includes an oligo. In embodiments, the blocking element is an oligo. In embodiments, the blocking element is an oligonucleotide having 5-25 nucleotides. In embodiments, the blocking element is an oligonucleotide having 10-50 nucleotides. In embodiments, the blocking element is an oligonucleotide having 20-75 nucleotides. In embodiments, the blocking element is an oligonucleotide having about 5, about 10, about 20, about 25, about 50, or about 75 nucleotides. In embodiments, the blocking element is a non-extendable oligomer. In embodiments, the blocking element includes two or more tandemly arranged oligos. In embodiments, the blocking element includes an oligonucleotide and an oligonucleotide that is the reverse complement of that oligonucleotide, or the partial reverse complement (e.g. creating a pair of partially overlapping oligonucleotides). In embodiments, the blocking element is a single-stranded oligonucleotide having a 5′ end and a 3′ end. In embodiments, the blocking element includes a 3′-blocked oligo. In embodiments, the blocking element includes a blocking moiety on the 3′ nucleotide. A blocking moiety on a nucleotide can be reversible, whereby the blocking moiety can be removed or modified to allow the 3′ hydroxyl to form a covalent bond with the 5′ phosphate of another nucleotide. For example, a reversible terminator may refer to a blocking moiety located, for example, at the 3′ position of the nucleotide and may be a chemically cleavable moiety such as an allyl group, an azidomethyl group or a methoxymethyl group, or may be an enzymatically cleavable group such as a phosphate ester. In embodiments, the blocking moiety is not reversible (e.g., the blocking element including a blocking moiety irreversibly prevents extension).

In embodiments, the blocking element is a non-extendable oligonucleotide. As described in US2010/0167353, blocking groups are known in the art that can be placed at or near the 3′ end of the oligonucleotide (e.g., a primer) to prevent extension. A primer or other oligonucleotide may be modified at the 3′-terminal nucleotide to prevent or inhibit initiation of DNA synthesis by, for example, the addition of a 3′ deoxyribonucleotide residue (e.g., cordycepin), a 2′,3′-dideoxyribonucleotide residue, non-nucleotide linkages or alkane-diol modifications (see, for example, U.S. Pat. No. 5,554,516). Alkane diol modifications which can be used to inhibit or block primer extension have also been described by Wilk et al., (1990 Nucleic Acids Res. 18 (8):2065), and by Arnold et al. (U.S. Pat. No. 6,031,091). Additional examples of suitable blocking groups include 3′ hydroxyl substitutions (e.g., 3′-phosphate, 3′-triphosphate or 3′-phosphate diesters with alcohols such as 3-hydroxypropyl), 2′,3′-cyclic phosphate, 2′ hydroxyl substitutions of a terminal RNA base (e.g., phosphate or sterically bulky groups such as triisopropyl silyl (TIPS) or tert-butyl dimethyl silyl (TBDMS)). 2′-alkyl silyl groups such as TIPS and TBDMS substituted at the 3′-end of an oligonucleotide are described in US 2007/0218490, which is incorporated herein by reference. Bulky substituents can also be incorporated on the base of the 3′-terminal residue of the oligonucleotide to block primer extension.

In embodiments, the blocking element includes an oligo having a 3′ dideoxynucleotide or similar modification to prevent extension by a polymerase and is used in conjunction with a non-strand displacing polymerase. In some embodiments, the blocking oligomer contains one or more non-natural bases that facilitate hybridization of the blocker to the target sequence (e.g., LNA bases). In some embodiments, the blocking oligomer contains other modified bases to increase resistance to exonuclease digestion (e.g., one or more phosphorothioate bonds). In embodiments, the blocking element is an oligonucleotide including one or more modified nucleotides, such as iso dGTP or iso dCTP, which are complementary to each other. In a reaction of polymerization lacking the complementary modified nucleotides, extension is blocked. In another embodiment, the blocking element is an oligonucleotide including a 3′ cleavable linker containing PEG, thereby blocking extension. In another embodiment, the blocking element is an oligonucleotide including one or more sequences which are recognized and bound by one or more short RNA or PNA oligos, thereby blocking the extension by a strand displacing DNA polymerase that cannot strand displace RNA or PNA. In embodiments, the blocking element is a modified nucleotide (e.g., a nucleotide including a reversible terminator, such as a 3′-reversible terminating moiety).

In embodiments, the blocking element includes an oligo, a protein, or a combination thereof. In embodiments, the blocking element includes a protein. In embodiments, the blocking element includes one or more proteins. The blocking element need not be an oligomer; in some embodiments, for example, the blocking element is a protein that selectively binds to the target sequence and prevents polymerase extension. In embodiments, the blocking element is an oligonucleotide including one or more modified nucleotides. In embodiments, the blocking element is an oligonucleotide including one or more modified nucleotides, wherein one or more modified nucleotides is linked to biotin, to which a protein (e.g., streptavidin) can be bound, thereby blocking polymerase extension. In embodiments, the blocking element includes one or more sequences which is recognized and bound by one or more single-stranded DNA-binding proteins, thereby blocking polymerase extension at the bound site.

In embodiments, the blocking element includes a CRISPR-Cas9 complex. For example, using a guide RNA specifically targeting the non-fusion sequence is introduced into a sample containing circularized ssDNA. The CRISPR-Cas9 complex then targets and cleaves the non-fusion sequence present in any circular ssDNA molecules. Following linearization by the CRISPR complex of the non-fusion circular ssDNA molecules, exonuclease digestion could then be performed to digest away the linear ssDNA molecules, enriching for those circular ssDNA molecules containing a fusion gene (e.g., lacking the non-fusion gene sequence targeted by the guide RNA). In embodiments, binding the blocking element includes forming a CRISPR-Cas9 complex with a guide RNA molecule bound to said non-fusion circular template polynucleotide.

In embodiments, the blocking element includes a biotin. For example, following circularization, the biotinylated blocking element is hybridized to the non-fusion gene sequence(s). The circular ssDNA molecules hybridized to the biotinylated blocking elements would then be pulled down using, for example, streptavidin-coated magnetic beads, depleting the sample of any non-fusion containing circular molecules prior to amplification.

In embodiments, the blocking element includes a restriction site. For example, the blocking element is used as a splint to enable restriction enzyme-mediated digestion of non-fusion containing circular ssDNA molecules into linear fragments that are not amplifiable. A methylated blocking oligomer could be used in combination with a methylation sensitive restriction enzyme (e.g., NotI, NaeI, NsbI, SalI, HapII, or HaeII).

In embodiments, binding the blocking element includes binding the blocking element upstream of the first primer. The terms “upstream” and “downstream” are used in accordance with their ordinary meaning in the art and refers to position(s) towards the 5′ end (upstream) or position(s) toward the 3′ end (downstream) in reference to a nucleic acid. In embodiments, the blocking element binds about 1 to 150 nucleotides upstream relative to the first primer. In embodiments, the blocking element binds about 1 to 15 nucleotides upstream relative to the first primer. In embodiments, the blocking element binds about 10 to about 25 nucleotides upstream relative to the first primer.

In embodiments, the method further includes binding a second blocking element downstream relative to the second primer on the one or more non-fusion circular template polynucleotides. In embodiments, the second blocking element binds about 100 to about 300 nucleotides downstream relative to the second primer. In embodiments, the second blocking element binds about 75 to about 150 nucleotides downstream relative to the second primer. In embodiments, the second blocking element binds about 50 to about 300 nucleotides downstream relative to the second primer. In embodiments, the second blocking element binds about 100 to about 400 nucleotides downstream relative to the second primer. In embodiments, the second blocking element binds about 100 to about 400 nucleotides downstream relative to the second primer.

In embodiments, the number (i.e., quantity) of fusion gene amplification products is about 1%, about 5%, about 10%, about 15%, about 20%, about 25%, about 30%, about 40%, about 50%, or about 75% more than the number (i.e., quantity) of non-fusion polynucleotide amplification products. In embodiments, the number (i.e., quantity) of fusion gene amplification products is about 1% more than the number (i.e., quantity) of non-fusion polynucleotide amplification products. In embodiments, the number (i.e., quantity) of fusion gene amplification products is about 5% more than the number (i.e., quantity) of non-fusion polynucleotide amplification products. In embodiments, the number (i.e., quantity) of fusion gene amplification products is about 10% more than the number (i.e., quantity) of non-fusion polynucleotide amplification products. In embodiments, the number (i.e., quantity) of fusion gene amplification products is about 15% more than the number (i.e., quantity) of non-fusion polynucleotide amplification products. In embodiments, the number (i.e., quantity) of fusion gene amplification products is about 20% more than the number (i.e., quantity) of non-fusion polynucleotide amplification products. In embodiments, the number (i.e., quantity) of fusion gene amplification products is about 25% more than the number (i.e., quantity) of non-fusion polynucleotide amplification products. In embodiments, the number (i.e., quantity) of fusion gene amplification products is about 30% more than the number (i.e., quantity) of non-fusion polynucleotide amplification products. In embodiments, the number (i.e., quantity) of fusion gene amplification products is about 40% more than the number (i.e., quantity) of non-fusion polynucleotide amplification products. In embodiments, the number (i.e., quantity) of fusion gene amplification products is about 50% more than the number (i.e., quantity) of non-fusion polynucleotide amplification products.

In embodiments, the number (i.e., quantity) of fusion gene amplification products is about 2-fold, at least about 1.5-fold, at least about 2.0-fold, at least about 2.5-fold, at least about 5-fold, at least about 10-fold, or more than about 10-fold greater the number (i.e., quantity) of non-fusion polynucleotide amplification products. In embodiments, the number (i.e., quantity) of fusion gene amplification products is about 2-fold greater the number (i.e., quantity) of non-fusion polynucleotide amplification products. In embodiments, the number (i.e., quantity) of fusion gene amplification products is at least about 1.5-fold greater the number (i.e., quantity) of non-fusion polynucleotide amplification products. In embodiments, the number (i.e., quantity) of fusion gene amplification products is at least about 2-fold greater the number (i.e., quantity) of non-fusion polynucleotide amplification products. In embodiments, the number (i.e., quantity) of fusion gene amplification products is at least about 2.5-fold greater the number (i.e., quantity) of non-fusion polynucleotide amplification products. In embodiments, the number (i.e., quantity) of fusion gene amplification products is at least about 5-fold greater the number (i.e., quantity) of non-fusion polynucleotide amplification products. In embodiments, the number (i.e., quantity) of fusion gene amplification products is at least about 10-fold greater the number (i.e., quantity) of non-fusion polynucleotide amplification products. In embodiments, the number (i.e., quantity) of fusion gene amplification products is more than about 10-fold greater the number (i.e., quantity) of non-fusion polynucleotide amplification products.

In embodiments, the one or more linear nucleic acid molecules include DNA, RNA, or cDNA; optionally wherein the DNA or the RNA are cell-free nucleic acid molecules. In embodiments, the one or more linear nucleic acid molecules include cfDNA, or isolated DNA, from a formalin fixed paraffin-embedded (FFPE) sample. In embodiments, the one or more linear nucleic acid molecules include RNA or cDNA, and the fusion junction includes an exon junction. In embodiments, the one or more linear nucleic acid molecules include cDNA, and the fusion junction includes an exon junction. In embodiments, the one or more linear nucleic acid molecules include RNA, and the fusion junction includes an exon junction. In embodiments, the one or more linear nucleic acid molecules include DNA, and the fusion junction includes an exon junction. In embodiments, the one or more linear nucleic acid molecules includes a sample barcode sequence, a molecular identifier sequence, or both a sample barcode sequence and a molecular identifier sequence. In embodiments, the fusion linear nucleic acid molecule is genomic DNA. In embodiments, the fusion linear nucleic acid molecule is a cell-free nucleic acid molecule. In embodiments, the fusion linear nucleic acid molecule is derived from a formalin fixed paraffin-embedded (FFPE) sample. In embodiments, the fusion linear nucleic acid molecule is derived from Hodgkin and Reed-Sternberg (HRS) cells. Reed-Sternberg cells are large, abnormal lymphocytes (a type of white blood cell) that may contain more than one nuclei. HRS cells are hallmark tumor cells of Hodgkin lymphoma. Reed-Sternberg cells are also called Hodgkin and Reed-Sternberg cells, as described in Marafioti et al. Blood (2000) 95 (4): 1443-1450, which is incorporated herein by reference.

In embodiments, the fusion gene includes an interchromosomal translocation (e.g., a fusion joining portions of two different chromosomes) or an intrachromosomal translocation (e.g., a fusion joining portions of the same chromosome). In embodiments, the fusion gene includes an interchromosomal translocation. In embodiments, the fusion gene includes an intrachromosomal translocation. In embodiments, the intrachromosomal translocation includes a partially or fully rearranged B cell or T cell antigen receptor. In embodiments, the intrachromosomal translocation includes a partially rearranged B cell antigen receptor. In embodiments, the intrachromosomal translocation includes a partially rearranged T cell antigen receptor. In embodiments, the intrachromosomal translocation includes a fully rearranged B cell antigen receptor. In embodiments, the intrachromosomal translocation includes a fully rearranged T cell antigen receptor.

In embodiments, the first primer hybridizes to the one or more fusion circular template polynucleotides about 1 to 100 nucleotides downstream relative to the fusion junction within the fusion gene. In embodiments, the first primer hybridizes to the one or more fusion circular template polynucleotides about 10 to about 50 nucleotides downstream relative to the fusion junction within the fusion gene. In embodiments, the first primer hybridizes to the one or more fusion circular template polynucleotides about 50 to about 200 nucleotides downstream relative to the fusion junction within the fusion gene. In embodiments, the first primer hybridizes to the one or more fusion circular template polynucleotides about 50 to about 100 nucleotides downstream relative to the fusion junction within the fusion gene. In embodiments, the first primer hybridizes to the one or more fusion circular template polynucleotides about 25 to about 50 nucleotides downstream relative to the fusion junction within the fusion gene. In embodiments, the first primer hybridizes to the one or more fusion circular template polynucleotides about 50 nucleotides downstream relative to the fusion junction within the fusion gene. In embodiments, the first primer hybridizes to the one or more fusion circular template polynucleotides about 25 nucleotides downstream relative to the fusion junction within the fusion gene. In embodiments, the first primer hybridizes to the one or more fusion circular template polynucleotides about 10 nucleotides downstream relative to the fusion junction within the fusion gene.

In embodiments, the first primer and the second primer hybridize to complementary sequences of the one or more fusion circular template polynucleotides, wherein the first primer and the second primer are separated by about 1 to about 50 nucleotides. In embodiments, the first primer and the second primer hybridize to complementary sequences of the one or more fusion circular template polynucleotides, wherein the first primer and the second primer are separated by about 1 to about 10 nucleotides. In embodiments, the first primer and the second primer hybridize to complementary sequences of the one or more fusion circular template polynucleotides, wherein the first primer and the second primer are separated by about 5 to about 25 nucleotides.

In embodiments, the first primer and the second primer hybridize to complementary sequences of the one or more fusion circular template polynucleotides and the one or more non-fusion circular template polynucleotides, wherein the first primer and the second primer are separated by about 1 to about 50 nucleotides. In embodiments, the first primer and the second primer hybridize to complementary sequences of the one or more fusion circular template polynucleotides and the one or more non-fusion circular template polynucleotides, wherein the first primer and the second primer are separated by about 1 to about 10 nucleotides. In embodiments, the first primer and the second primer hybridize to complementary sequences of the one or more fusion circular template polynucleotides and the one or more non-fusion circular template polynucleotides, wherein the first primer and the second primer are separated by about 5 to about 25 nucleotides. In embodiments, the first and second primer do not share overlapping complementary sequences. In embodiments, the first primer and the second primer are separated by about 10 nucleotides. In embodiments, the first primer and the second primer are separated by about 25 nucleotides. In embodiments, the first primer and the second primer are separated by about 50 nucleotides. In embodiments, the first primer and the second primer are separated by about 75 nucleotides. In embodiments, the first primer and the second primer are separated by about 100 nucleotides.

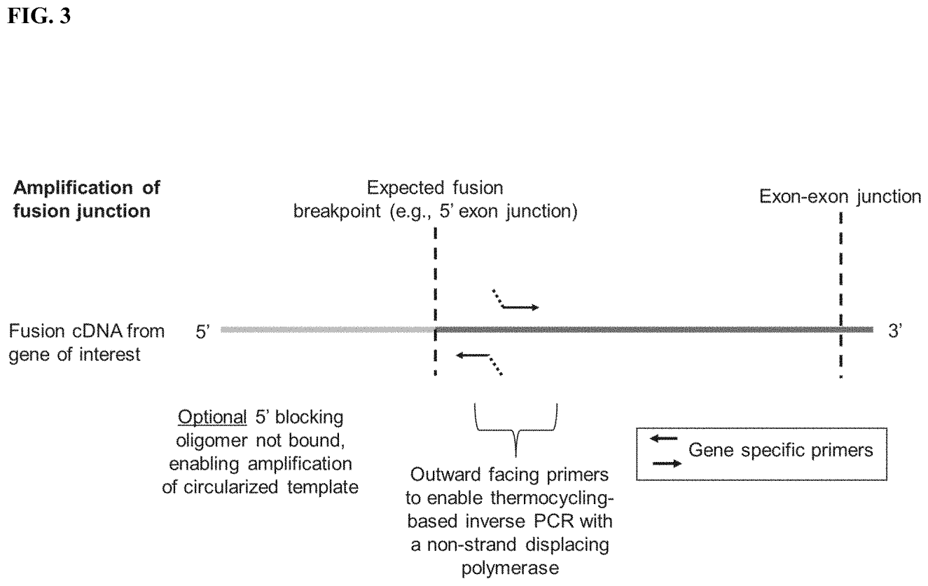

In embodiments, the first primer and the second primer are outward facing primers. In embodiments, the first primer and the second primer are an outward facing inverse PCR primer pair. Outward facing primers are designed such that amplification only occurs on a circular template polynucleotide, for example, as in inverse PCR (see, Ochman H et al. Genetic. 1988; 120(3):621-3 and Willis T G et al. Blood. 1997; 90(6): 2456-64, each of which is incorporated herein by reference). Inverse PCR (as illustrated in FIGS. 2A-2B and FIG. 3) (also known as inverted or inside-out PCR) is used to amplify DNA sequences that flank one end of a known DNA sequence and for which no primers are typically available. The unknown sequence is amplified by two primers that bind specifically to the known sequence and point in opposite directions (i.e., outward facing primers). In embodiments, the product(s) of the inverse PCR amplification reaction is a linear DNA fragment.

In embodiments, the second number of fusion polynucleotide amplification products is about 1%, about 5%, about 10%, about 15%, about 20%, about 25%, about 30%, about 40%, about 50%, about 75% more than the first number of non-fusion polynucleotide amplification products. In embodiments, the second number of fusion polynucleotide amplification products is about 0.01%, about 0.05%, about 0.010%, about 0.015%, about 0.020%, about 0.025%, about 0.030%, about 0.040%, about 0.050%, about 0.075% more than the first number of non-fusion polynucleotide amplification products. In embodiments, the second number of fusion polynucleotide amplification products is about 0.1%, about 0.5%, about 0.10%, about 0.15%, about 0.20%, about 0.25%, about 0.30%, about 0.40%, about 0.50%, about 0.75% more than the first number of non-fusion polynucleotide amplification products. In embodiments, the second number of fusion polynucleotide amplification products is greater than the first number of non-fusion polynucleotide amplification products. In embodiments, the first number of non-fusion polynucleotide amplification products is about 1%, about 5%, about 10%, about 15%, about 20%, about 25%, about 30%, about 40%, about 50%, about 75% less than the second number of fusion polynucleotide amplification products. In embodiments, the first number of non-fusion polynucleotide amplification products is about 0.01%, about 0.05%, about 0.010%, about 0.015%, about 0.020%, about 0.025%, about 0.030%, about 0.040%, about 0.050%, about 0.075% less than the second number of fusion polynucleotide amplification products. In embodiments, the first number of non-fusion polynucleotide amplification products is about 0.1%, about 0.5%, about 0.10%, about 0.15%, about 0.20%, about 0.25%, about 0.30%, about 0.40%, about 0.50%, about 0.75% less than the second number of fusion polynucleotide amplification products.

In embodiments, the second number of fusion polynucleotide amplification products is about 2-fold, at least about 1.5-fold, at least about 2.0-fold, at least about 2.5-fold, at least about 5-fold, at least about 10-fold, or more than about 10-fold greater than the first number of non-fusion polynucleotide amplification products. In embodiments, the second number of fusion polynucleotide amplification products is about 1.0-fold greater than the first number of non-fusion polynucleotide amplification products. In embodiments, the second number of fusion polynucleotide amplification products is about 2.0-fold greater than the first number of non-fusion polynucleotide amplification products. In embodiments, the second number of fusion polynucleotide amplification products is about 5.0-fold greater than the first number of non-fusion polynucleotide amplification products. In embodiments, the second number of fusion polynucleotide amplification products is about 10-fold greater than the first number of non-fusion polynucleotide amplification products. In embodiments, the second number of fusion polynucleotide amplification products is more than 10-fold greater than the first number of non-fusion polynucleotide amplification products. In embodiments, the second number of fusion polynucleotide amplification products is about 20-fold greater than the first number of non-fusion polynucleotide amplification products.

In embodiments, the second number quantified after one cycle of extension is measurably higher than the first number. In embodiments, the method generates a first number of non-fusion polynucleotide amplification products and a second number of fusion polynucleotide amplification products at a ratio of 1.00:1.01. In embodiments, the ratio of first number to second number is 1.00:1.02. In embodiments, the ratio of first number to second number is 1.00:1.05. In embodiments, the ratio of first number to second number is 1.00:1.10. Following 35 extension cycles (e.g., 35 PCR cycles, wherein each cycle includes the steps of primer hybridization, primer extension, and denaturation), a ratio of 1.00:1.02 yields a fold enrichment of 1.0235 of about 1.999-fold enrichment of the second number relative to the first number. In embodiments, the second number quantified after a plurality of extension cycles (e.g., 5, 10, 15, 20) is measurably higher than the first number. In embodiments, the second number quantified after 1, 2, 3, 4, 5, 10, 15, or 20 minutes of amplification (e.g., eRCA) is measurably higher than the first number.

In embodiments, the one or more linear nucleic acid molecules are about 20 to about 1000 nucleotides in length, about 100 to about 300 nucleotides in length, about 300 to about 500 nucleotides in length, or about 500 to about 1000 nucleotides in length. In embodiments, the one or more linear nucleic acid molecules are about 20 to 1000 nucleotides in length. In embodiments, the one or more linear nucleic acid molecules are about 100 to about 300 nucleotides in length. In embodiments, the one or more linear nucleic acid molecules are about 300 to about 500 nucleotides in length. In embodiments, the one or more linear nucleic acid molecules are about 500 to about 1000 nucleotides in length. In embodiments, the one or more linear nucleic acid molecules are about 20, about 50, about 75, about 100, about 150, about 200, about 250, about 300, about 350, about 400, about 450, about 500, about 550, about 600, about 650, about 700, about 750, about 800, about 850, about 900, about 950, or about 1000 nucleotides in length. In embodiments, the linear nucleic acid molecules are about 50 to about 1000, 100 to about 800, or about 200 to about 600 nucleotides in length.

In embodiments, the linear molecules are derived from a biological sample. In embodiments, the linear molecules are derived from a sample. In embodiments, the linear molecules are derived from a diseased patient. In embodiments, the linear molecules are derived from a cancer patient. “Patient” refers to a living organism (i.e., a subject) suffering from, or prone to, a disease or condition. Non-limiting examples include humans, other mammals, bovines, rats, mice, dogs, monkeys, goat, sheep, cows, deer, and other non-mammalian animals. In some embodiments, the patient is human.

In embodiments, the one or more linear nucleic acid molecules include DNA, RNA, or cDNA; optionally wherein the DNA or the RNA are cell-free nucleic acid molecules. In embodiments, the one or more linear nucleic acid molecules include RNA or cDNA, and the fusion junction is at an exon junction. In embodiments, the one or more linear nucleic acid molecules include RNA or cDNA, and the fusion gene includes an exon junction formed by alternative splicing. In embodiments, the one or more linear nucleic acid molecules include RNA or cDNA, and the fusion gene includes an exon junction formed from a splicing defect.

In embodiments, the one or more linear nucleic acid molecules include a barcode sequence. In embodiments, a plurality of linear nucleic acid molecules (e.g., all linear nucleic acid molecules from a particular sample source, or sub-sample thereof) are joined to a first barcode sequence, while a different plurality of linear nucleic acid molecules (e.g., all linear nucleic acid molecules from a different sample source, or different subsample) are joined to a second barcode sequence, thereby associating each plurality of linear nucleic acid molecules with a different barcode sequence indicative of sample source. In embodiments, each barcode sequence in a plurality of barcode sequences differs from every other barcode sequence in the plurality by at least three nucleotide positions, such as at least 3, 4, 5, 6, 7, 8, 9, 10, or more nucleotide positions. In some embodiments, substantially degenerate barcode sequences may be known as random. In some embodiments, a barcode sequence may include a nucleic acid sequence from within a pool of known sequences. In some embodiments, the barcode sequence may be pre-defined. In embodiments, the barcode sequence includes about 1 to about 10 nucleotides. In embodiments, the barcode sequence includes about 3, 4, 5, 6, 7, 8, 9, or about 10 nucleotides. In embodiments, the barcode sequence includes about 3 nucleotides. In embodiments, the barcode sequence includes about 5 nucleotides. In embodiments, the barcode sequence includes about 7 nucleotides. In embodiments, the barcode sequence includes about 10 nucleotides. In embodiments, the barcode sequence includes about 6 to about 10 nucleotides.

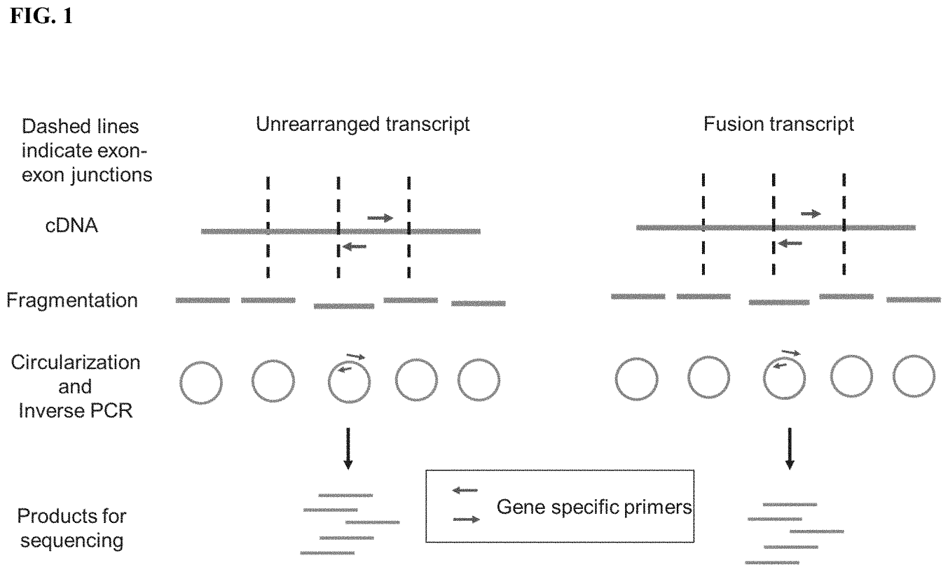

FIG. 1 and Example 1 describe an example of how cDNA can be fragmented to generate linear nucleic acid molecules. In embodiments, prior to circularizing one or more linear nucleic acid molecules, the polynucleotide is fragmented to an average length of approximately 150, approximately 250, or approximately 350 base pairs. Fragmentation may be accomplished via methods known in the art (e.g., enzymatic fragmentation, acoustic fragmentation). In embodiments, the polynucleotide is fragmented to generate linear nucleic acid molecules using enzymatic fragmentation or acoustic fragmentation. In embodiments, the input polynucleotide is derived from a fresh or fresh frozen sample and is minimally degraded prior to fragmentation. Next, ssDNA fragments are circularized via CircLigase™ or a method described herein. In some embodiments, circularization is facilitated by denaturing nucleic acids prior to circularization. Residual linear DNA molecules may be optionally digested. This may be accomplished via methods known in the art (e.g., treating with Exo I and/or Exo III enzymes).

In embodiments, the circularizing includes intramolecular joining of the 5′ and 3′ ends of a linear nucleic acid molecule. In embodiments, the circularizing includes a ligation reaction. In embodiments, the two ends of the linear nucleic acid molecule are ligated directly together. In embodiments, the two ends of the linear nucleic acid molecule are ligated together with the aid of a bridging oligonucleotide (sometimes referred to as a splint oligonucleotide) that is complementary with the two ends of the linear nucleic acid molecule. Methods for forming circular DNA templates are known in the art, for example, linear polynucleotides are circularized in a non-template driven reaction with circularizing ligase, such as CircLigase™, CircLigase™ II, Taq DNA Ligase, HiFi Taq DNA Ligase, T4 DNA ligase, or Ampligase® DNA Ligase. In some embodiments, circularization is facilitated by denaturing double-stranded linear nucleic acids prior to circularization. Residual linear DNA molecules may be optionally digested. In some embodiments, circularization is facilitated by chemical ligation (e.g., click chemistry, e.g., a copper-catalyzed reaction of an alkyne (e.g., a 3′ alkyne) and an azide (e.g., a 5′ azide)). In embodiments, prior to circularization, the linear DNA fragments are A-tailed (e.g., A-tailed using Taq DNA polymerase).

In embodiments, circularization of the linear nucleic acid molecule is performed with CircLigase™ enzyme. In embodiments, circularization of the linear nucleic acid molecule is performed with a thermostable RNA ligase, or mutant thereof. In embodiments, circularization of the linear nucleic acid molecule is performed with an RNA ligase enzyme from bacteriophage TS2126, or mutant thereof. For example, the RNA ligase may be TS2126 RNA ligase, as described in U.S. Pat. Pub. 2005/0266439, which is incorporated herein by reference in its entirety.

In embodiments, circularization includes contacting a double-stranded polynucleotide with at least one protelomerase. In embodiments, the double-stranded polynucleotide includes complementary protelomerase target sequences at both ends (e.g., the 5′ and 3′ end of each strand includes a protelomerase recognition sequence, or complement thereof). For example, both ends of the target double-stranded DNA molecule are inserted with the double-stranded enzyme recognition DNA molecule (e.g., the double-stranded protelomerase recognition sequence, for example a TeIN protelomerase recognition sequence, has been ligated to each end of the dsDNA molecule). Then, for example, the Escherichia coli phage N15 protelomerase (TelN) catalyzes the double-stranded enzyme recognition DNA molecule on both ends of the target double-stranded DNA molecule to produce a circularized DNA molecule with the target double-stranded DNA molecule circularized. The TelN recognition sequence is TATCAGCACACAATTGCCCATTATACGCGCGTATAATGGACTATTGTGTGCTGATA (SEQ ID NO: 1). TelN cleaves this sequence at its mid-point and joins the ends of the complementary strands to form covalently closed ends. Additional methods for protelomerase circularization and protelomerase enzymes are disclosed in PCT Pat. Pubs. WO2021236792 and WO2021/078947, and U.S. Pat. Pub. 2013/0216562, each of which is incorporated herein by reference in its entirety.

In embodiments, circularizing includes ligating a first hairpin and a second hairpin adapter to a linear nucleic acid molecule, thereby forming a circular polynucleotide.

In embodiments, a hairpin adapter includes a single nucleic acid strand including a stem-loop structure. A hairpin adapter can be any suitable length. In some embodiments, a hairpin adapter is at least 40, at least 50, or at least 100 nucleotides in length. In some embodiments, a hairpin adapter has a length in a range of 45 to 500 nucleotides, 75-500 nucleotides, 45 to 250 nucleotides, 60 to 250 nucleotides or 45 to 150 nucleotides. In some embodiments, a hairpin adapter includes a nucleic acid having a 5′-end, a 5′-portion, a loop, a 3′-portion and a 3′-end (e.g., arranged in a 5′ to 3′ orientation). In some embodiments, the 5′ portion of a hairpin adapter is annealed and/or hybridized to the 3′ portion of the hairpin adapter, thereby forming a stem portion of the hairpin adapter. In some embodiments, the 5′ portion of a hairpin adapter is substantially complementary to the 3′ portion of the hairpin adapter. In certain embodiments, a hairpin adapter includes a stem portion (i.e., stem) and a loop, wherein the stem portion is substantially double stranded thereby forming a duplex. In some embodiments, the loop of a hairpin adapter includes a nucleic acid strand that is not complementary (e.g., not substantially complementary) to itself or to any other portion of the hairpin adapter. In some embodiments, the second adapter includes a sample barcode sequence, a molecular identifier sequence, or both a sample barcode sequence and a molecular identifier sequence. In some embodiments, the second adapter includes a sample barcode sequence.

In some embodiments, a duplex region or stem portion of a hairpin adapter includes an end that is configured for ligation to an end of double stranded nucleic acid (e.g., a nucleic acid fragment, e.g., a library insert). In embodiments, an end of a duplex region or stem portion of a hairpin adapter includes a 5′-overhang or a 3′-overhang that is complementary to a 3′-overhang or a 5′-overhang of one end of a double stranded nucleic acid. In some embodiments, an end of a duplex region or stem portion of a hairpin adapter includes a blunt end that can be ligated to a blunt end of a double stranded nucleic acid. In certain embodiment, an end of a duplex region or stem portion of a hairpin adapter includes a 5′-end that is phosphorylated. In some embodiments, a stem portion of a hairpin adapter is at least 15, at least 25, or at least 40 nucleotides in length. In some embodiments, a stem portion of a hairpin adapter has a length in a range of 15 to 500 nucleotides, 15-250 nucleotides, 15 to 200 nucleotides, 15 to 150 nucleotides, 20 to 100 nucleotides or 20 to 50 nucleotides.

In some embodiments, the loop of a hairpin adapter includes one or more of a primer binding site, a capture nucleic acid binding site (e.g., a nucleic acid sequence complementary to a capture nucleic acid), a UMI, a sample barcode, a sequencing adapter, a label, the like or combinations thereof. In certain embodiments, a loop of a hairpin adapter includes a primer binding site. In certain embodiments, a loop of a hairpin adapter includes a primer binding site and a UMI. In certain embodiments, a loop of a hairpin adapter includes a binding motif.

In some embodiments, the loop of a hairpin adapter has a predicted, calculated, mean, average or absolute melting temperature (Tm) that is greater than 50° C., greater than 55° C., greater than 60° C., greater than 65° C., greater than 70° C. or greater than 75° C. In some embodiments, a loop of a hairpin adapter has a predicted, estimated, calculated, mean, average or absolute melting temperature (Tm) that is in a range of 50-100° C., 55-100° C., 60-100° C., 65-100° C., 70-100° C., 55-95° C., 65-95° C., 70-95° C., 55-90° C., 65-90° C., 70-90° C., or 60-85° C. In embodiments, the Tm of the loop is about 65° C. In embodiments, the Tm of the loop is about 75° C. In embodiments, the Tm of the loop is about 85° C. The Tm of a loop of a hairpin adapter can be changed (e.g., increased) to a desired Tm using a suitable method, for example by changing (e.g., increasing GC content), changing (e.g., increasing) length and/or by the inclusion of modified nucleotides, nucleotide analogues and/or modified nucleotides bonds, non-limiting examples of which include locked nucleic acids (LNAs, e.g., bicyclic nucleic acids), bridged nucleic acids (BNAs, e.g., constrained nucleic acids), CS-modified pyrimidine bases (for example, 5-methyl-dC, propynyl pyrimidines, among others) and alternate backbone chemistries, for example peptide nucleic acids (PNAs), morpholinos, the like or combinations thereof. Accordingly, in some embodiments, a loop of a hairpin adapter includes one or more modified nucleotides, nucleotide analogues and/or modified nucleotides bonds.

In some embodiments, the loop of a hairpin adapter independently includes a GC content of greater than 40%, greater than 50%, greater than 55%, greater than 60% greater than 65% or greater than 70%. In certain embodiments, a loop of a hairpin adapter independently includes a GC content in a range of 40-100%, 50-100%, 60-100% or 70-100%. In embodiments, the loop has a GC content of about or more than about 40%. In embodiments, the loop has a GC content of about or more than about 50%. In embodiments, the loop has a GC content of about or more than about 60%. Non-base modifiers can also be incorporated into a loop of a hairpin adapter to increase Tm, non-limiting examples of which include a minor grove binder (MGB), spermine, G-clamp, a Uaq anthraquinone cap, the like or combinations thereof. A loop of a hairpin adapter can be any suitable length. In some embodiments, a loop of a hairpin adapter is at least 15, at least 25, or at least 40 nucleotides in length. In some embodiments, a hairpin adapter has a length in a range of 15 to 500 nucleotides, 15-250 nucleotides, 20 to 200 nucleotides, 30 to 150 nucleotides or 50 to 100 nucleotides.

In certain embodiments, a duplex region or stem region of a hairpin adapter includes a predicted, estimated, calculated, mean, average or absolute Tm in a range of 30-70° C., 35-65° C., 35-60° C., 40-65° C., 40-60° C., 35-55° C., 40-55° C., 45-50° C. or 40-50° C. In embodiments, the Tm of the stem region is about or more than about 35° C. In embodiments, the Tm of the stem region is about or more than about 40° C. In embodiments, the Tm of the stem region is about or more than about 45° C. In embodiments, the Tm of the stem region is about or more than about 50° C.

In embodiments, circularizing includes hybridizing a splint to both ends of a linear nucleic acid molecule and i) ligating the adjacent ends or ii) extending the 3′ end of the linear nucleic acid molecule along the splint to generate a complementary sequence of the splint and ligating the 3′ end of the complementary sequence to the 5′ end of the linear nucleic acid molecule. In embodiments, the splint includes a barcode. In embodiments, the splint includes a primer binding site (e.g., a sequence complementary to an amplification or sequencing primer).

In one embodiment, an enzyme is used to ligate the two ends of the linear nucleic acid molecule. For example, linear polynucleotides are circularized in a non-template driven reaction with a circularizing ligase, such as CircLigase™ enzyme, Taq DNA Ligase, HiFi Taq DNA Ligase, T4 ligase, PBCV-1 DNA Ligase (also known as SplintR ligase) or Ampligase DNA Ligase). Non-limiting examples of ligases include DNA ligases such as DNA Ligase I, DNA Ligase II, DNA Ligase III, DNA Ligase IV, T4 DNA ligase, T7 DNA ligase, T3 DNA Ligase, E. coli DNA Ligase, PBCV-1 DNA Ligase (also known as SplintR ligase) or a Taq DNA Ligase. In embodiments, the ligase enzyme includes a T4 DNA ligase, T4 RNA ligase 1, T4 RNA ligase 2, T3 DNA ligase or T7 DNA ligase. In embodiments, the enzymatic ligation is performed by a mixture of ligases. In embodiments, the ligation enzyme is selected from the group consisting of T4 DNA ligase, T4 RNA ligase 1, T4 RNA ligase 2, RtcB ligase, T3 DNA ligase, T7 DNA ligase, Taq DNA ligase, PBCV-1 DNA Ligase, a thermostable DNA ligase (e.g., 5′AppDNA/RNA ligase), an ATP dependent DNA ligase, an RNA-dependent DNA ligase (e.g., SplintR ligase), and combinations thereof. In embodiments, the two ends of the template polynucleotide are ligated together with the aid of a splint primer that is complementary with the two ends of the template polynucleotide. For example, a T4 ligation reaction may be carried out by combining a linear polynucleotide, ligation buffer, ATP, T4 DNA ligase, water, and incubating the mixture at between about 20° C. to about 45° C., for between about 5 minutes to about 30 minutes. In some embodiments, the T4 ligation reaction is incubated at 37° C. for 30 minutes. In some embodiments, the T4 ligation reaction is incubated at 45° C. for 30 minutes. In embodiments, the ligase reaction is stopped by adding Tris buffer with high EDTA and incubating for 1 minute.

In embodiments, a linear nucleic acid molecule may undergo intramolecular circularization (via ligation or annealing) without joining to a circularization adapter (e.g., self-circularization). Circularization (without a circularization adaptor) can be achieved with a ligase at about 4°−35° C. In embodiments, a linear nucleic acid molecule interest can be joined to a loxP adapter and circularization can be mediated by a Cre recombinase enzyme reaction at about 4°-35° C., see for example U.S. Pat. No. 6,465,254, which is incorporated herein by reference.

In embodiments, the circular polynucleotide that is about 100 to about 1000 nucleotides in length, about 100 to about 300 nucleotides in length, about 300 to about 500 nucleotides in length, or about 500 to about 1000 nucleotides in length. In embodiments, the circular polynucleotide is about 300 to about 600 nucleotides in length. In embodiments, the circular polynucleotide is about 100-1000 nucleotides, about 150-950 nucleotides, about 200-900 nucleotides, about 250-850 nucleotides, about 300-800 nucleotides, about 350-750 nucleotides, about 400-700 nucleotides, or about 450-650 nucleotides in length. In embodiments, the circular polynucleotide molecule is about 100-1000 nucleotides in length. In embodiments, the circular polynucleotide molecule is about 100-300 nucleotides in length. In embodiments, the circular polynucleotide molecule is about 300-500 nucleotides in length. In embodiments, the circular polynucleotide molecule is about 500-1000 nucleotides in length. In embodiments, the circular polynucleotide molecule is about 100 nucleotides. In embodiments, the circular polynucleotide molecule is about 300 nucleotides. In embodiments, the circular polynucleotide molecule is about 500 nucleotides. In embodiments, the circular polynucleotide molecule is about 1000 nucleotides. Circular polynucleotides may be conveniently isolated by a conventional purification column, digestion of non-circular DNA by one or more appropriate exonucleases, or both.

In embodiments, the sequence that specifically binds the blocking element, the sequence that specifically hybridizes to the first primer, or both are about 1 to about 100 nucleotides from the fusion junction. In embodiments, the sequence that specifically binds the blocking element, the sequence that specifically hybridizes to the first primer, or both are about 5 to about 100 nucleotides from the fusion junction. In embodiments, the sequence that specifically binds the blocking element, the sequence that specifically hybridizes to the first primer, or both are about 10 to about 100 nucleotides from the fusion junction. In embodiments, the sequence that specifically binds the blocking element, the sequence that specifically hybridizes to the first primer, or both are about 25 to about 100 nucleotides from the fusion junction. In embodiments, the sequence that specifically binds the blocking element, the sequence that specifically hybridizes to the first primer, or both are about 50 to about 100 nucleotides from the fusion junction. In embodiments, the sequence that specifically binds the blocking element, the sequence that specifically hybridizes to the first primer, or both are about 75 to about 100 nucleotides from the fusion junction. In embodiments, the sequence that specifically binds the blocking element, the sequence that specifically hybridizes to the first primer, or both are about 1, about 5, about 10, about 25, about 50, about 75, or about 100 nucleotides from the fusion junction. In embodiments, the sequence that specifically hybridizes to the first primer and the sequence that specifically hybridizes to the blocking element do not overlap. In embodiments, the sequence that specifically hybridizes to the first primer and the sequence that specifically hybridizes to the blocking elements are about 5, about 10, or about 20 nucleotides apart. In embodiments, the sequence that specifically binds the blocking element and the sequence that specifically hybridizes to the first primer are about the same distance from the fusion junction. In embodiments, the sequence that specifically binds the blocking element and the sequence that specifically hybridizes to the first primer are different distances from the fusion junction.

In embodiments, the sequence that specifically hybridizes to the first primer and the sequence complementary to the sequence that specifically hybridizes to the second primer are separated by about 1 to about 50 nucleotides. In embodiments, the sequence that specifically hybridizes to the first primer and the sequence complementary to the sequence that specifically hybridizes to the second primer are separated by about 5 to about 50 nucleotides. In embodiments, the sequence that specifically hybridizes to the first primer and the sequence complementary to the sequence that specifically hybridizes to the second primer are separated by about 10 to about 50 nucleotides. In embodiments, the sequence that specifically hybridizes to the first primer and the sequence complementary to the sequence that specifically hybridizes to the second primer are separated by about 20 to about 50 nucleotides. In embodiments, the sequence that specifically hybridizes to the first primer and the sequence complementary to the sequence that specifically hybridizes to the second primer are separated by about 30 to about 50 nucleotides. In embodiments, the sequence that specifically hybridizes to the first primer and the sequence complementary to the sequence that specifically hybridizes to the second primer are separated by about 40 to about 50 nucleotides. In embodiments, the sequence that specifically hybridizes to the first primer and the sequence complementary to the sequence that specifically hybridizes to the second primer are separated by about 1, about 5, about 10, about 20, about 30, about 40, or about 50 nucleotides.

In embodiments, the sequence that specifically hybridizes to the first primer and the sequence complementary to the sequence that specifically hybridizes to the second primer are within the same exon of a target gene. In embodiments, the sequence that specifically hybridizes to the first primer and the sequence complementary to the sequence that specifically hybridizes to the second primer are within different exons of a target gene. In embodiments, the sequence that specifically hybridizes to the first primer and the sequence complementary to the sequence that specifically hybridizes to the second primer are neighboring exons of a target gene. Specific hybridization discriminates over non-specific hybridization interactions (e.g., two nucleic acids that a not configured to specifically hybridize, e.g., two nucleic acids that are 80% or less, 70% or less, 60% or less or 50% or less complementary) by about 2-fold or more, often about 10-fold or more, and sometimes about 100-fold or more, 1000-fold or more, 10,000-fold or more, 100,000-fold or more, or 1,000,000-fold or more. Two nucleic acid strands that are hybridized to each other can form a duplex which includes a double-stranded portion of nucleic acid.

In embodiments, the linear nucleic acid molecules are single-stranded nucleic acid molecules. In embodiments, the linear nucleic acid molecules are double-stranded nucleic acid molecules. In embodiments, the method includes less than 200 ng of linear nucleic acid molecules. In embodiments, the method includes less than 100 ng of linear nucleic acid molecules. In embodiments, the method includes less than 50 ng of linear nucleic acid molecules. In embodiments, the method includes less than 20 ng of linear nucleic acid molecules. In embodiments, the method includes less than 10 ng of linear nucleic acid molecules. In embodiments, the method includes about 200 ng of linear nucleic acid molecules. In embodiments, the method includes about 100 ng of linear nucleic acid molecules. In embodiments, the method includes about 50 ng of linear nucleic acid molecules. In embodiments, the method includes about 20 ng of linear nucleic acid molecules. In embodiments, the method includes about 10 ng of linear nucleic acid molecules.

In some embodiments, a double stranded nucleic acid includes two complementary nucleic acid strands. In certain embodiments, a double stranded nucleic acid includes a first strand and a second strand which are complementary or substantially complementary to each other. A first strand of a double stranded nucleic acid is sometimes referred to herein as a forward strand and a second strand of the double stranded nucleic acid is sometime referred to herein as a reverse strand. In some embodiments, a double stranded nucleic acid includes two opposing ends. Accordingly, a double stranded nucleic acid often includes a first end and a second end. An end of a double stranded nucleic acid may include a 5′-overhang, a 3′-overhang or a blunt end. In some embodiments, one or both ends of a double stranded nucleic acid are blunt ends. In certain embodiments, one or both ends of a double stranded nucleic acid are manipulated to include a 5′-overhang, a 3′-overhang or a blunt end using a suitable method. In some embodiments, one or both ends of a double stranded nucleic acid are manipulated during library preparation such that one or both ends of the double stranded nucleic acid are configured for ligation to an adapter using a suitable method. For example, one or both ends of a double stranded nucleic acid may be digested by a restriction enzyme, polished, end-repaired, filled in, phosphorylated (e.g., by adding a 5′-phosphate), dT-tailed, dA-tailed, the like or a combination thereof.

In embodiments, (i) the first primer includes a 5′ sequence that does not hybridize to the first strand of the first region under the amplification conditions; and/or (ii) the second primer includes a 5′ sequence that does not hybridize to a complement of the first strand of the first region under the amplification conditions. In embodiments, (i) the first primer includes a 5′ sequence that does not hybridize to the first strand of the first region under the amplification conditions; and (ii) the second primer includes a 5′ sequence that does not hybridize to a complement of the first strand of the first region under the amplification conditions. In embodiments, (i) the first primer includes a 5′ sequence that does not hybridize to the first strand of the first region under the amplification conditions; or (ii) the second primer includes a 5′ sequence that does not hybridize to a complement of the first strand of the first region under the amplification conditions. In some embodiments, the 5′ sequence of the first primer that does not hybridize to the first strand of the first region includes a primer binding site for a secondary amplification. In some embodiments, the 5′ sequence of the first primer that does not hybridize to the first strand of the first region includes a first sequencing adapter used for clustering of the template on a flow cell. In some embodiments, the 5′ sequence of the first primer that does not hybridize to the first strand of the first region includes a sample barcode. In some embodiments, the 5′ sequence of the second primer that does not hybridize to the complement of the first strand of the first region includes a primer binding site for a secondary amplification. In some embodiments, the 5′ sequence of the second primer that does not hybridize to the first strand of the first region includes a second sequencing adapter used for clustering of the template on a flow cell. In some embodiments, the 5′ sequence of the second primer that does not hybridize to the complement of the first strand of the first region includes a sample barcode.

In embodiments, (i) the amplification reaction further includes a second blocking element that inhibits polymerase extension along a sequence to which it binds, and (ii) the first region includes a first strand including from 5′ to 3′ the sequence complementary to a sequence that specifically hybridizes to the second primer, and a sequence complementary to a sequence that specifically binds to the second blocking element. In embodiments, the sequence complementary to a sequence that specifically hybridizes to the second primer and the sequence complementary to a sequence that specifically binds the second blocking element are separated by about 100 to about 300 nucleotides. In embodiments, the sequence complementary to a sequence that specifically hybridizes to the second primer and the sequence complementary to a sequence that specifically binds the second blocking element are separated by about 100 to about 200 nucleotides. In embodiments, the sequence complementary to a sequence that specifically hybridizes to the second primer and the sequence complementary to a sequence that specifically binds the second blocking element are separated by about 100 to about 150 nucleotides. In embodiments, the sequence complementary to a sequence that specifically hybridizes to the second primer and the sequence complementary to a sequence that specifically binds the second blocking element are separated by about 100, about 150, about 200, or about 300 nucleotides.

In embodiments, the method further includes: d) amplifying the one or more non-fusion circular template polynucleotides to generate a third number of non-fusion polynucleotide amplification products; and amplifying the one or more fusion circular template polynucleotides to generate a fourth number of fusion polynucleotide amplification products, wherein the third number and the fourth number are substantially the same. In embodiments, amplifying the one or more non-fusion circular template polynucleotides includes hybridizing a third primer and a fourth primer to the one or more non-fusion circular template polynucleotides and extending both primers with a polymerase, and wherein amplifying the one or more fusion circular template polynucleotides includes hybridizing a third primer and a fourth primer to the one or more fusion circular template polynucleotides and extending both primers with a polymerase. In embodiments, the third primer hybridizes upstream (e.g., in the 5′ direction) of a target sequence, and the fourth primer hybridizes downstream (e.g., in the 3′ direction) of a target sequence, wherein the target sequence includes a single-nucleotide variant, an insertion, a deletion, an internal tandem duplication, or a copy number variant. In embodiments, the target sequence includes one or more single-nucleotide variants, one or more insertions, one or more deletions, one or more internal tandem duplications, and/or one or more copy number variants.

In embodiments, the amplifying of circularized or linear polynucleotides includes a plurality of cycles including the steps of primer hybridization, primer extension, and denaturation in the presence of the first primer, the blocking element, and the second primer. Although each cycle will include each of these three events (hybridization, extension, and denaturation), events within a cycle may or may not be discrete. For example, each step may have different reagents and/or reaction conditions (e.g., temperatures). Alternatively, some steps may proceed without a change in reaction conditions. For example, extension may proceed under the same conditions (e.g., same temperature) as hybridization. After extension, the conditions are changed to start a new cycle with a new denaturation step, thereby amplifying the polynucleotide. Primer extension products from an earlier cycle may serve as templates for a later amplification cycle. In embodiments, the plurality of cycles is about 5 to about 50 cycles. In embodiments, the plurality of cycles is about 10 to about 45 cycles. In embodiments, the plurality of cycles is about 10 to about 20 cycles. In embodiments, the plurality of cycles is about 20 to about 30 cycles. In embodiments, the plurality of cycles is 10 to 45 cycles. In embodiments, the plurality of cycles is 10 to 20 cycles. In embodiments, the plurality of cycles is 20 to 30 cycles. In embodiments, the plurality of cycles is about 10 to about 45 cycles. In embodiments, the plurality of cycles is about 20 to about 30 cycles.

In embodiments, the amplifying includes exponentially amplifying the circular template polynucleotide including the fusion junction. In embodiments, the amplifying include exponential rolling circle amplification (eRCA). Exponential RCA is similar to the linear process except that it uses a second primer having a sequence that is identical to at least a portion of the circular template (Lizardi et al. Nat. Genet. 19:225 (1998)). This two-primer system achieves isothermal, exponential amplification. Exponential RCA has been applied to the amplification of non-circular DNA through the use of a linear probe that binds at both of its ends to contiguous regions of a target DNA followed by circularization using DNA ligase (Nilsson et al. Science 265(5181):208 5(1994)). In embodiments, the amplifying includes hyperbranched rolling circle amplification (HRCA). Hyperbranched RCA uses a second primer complementary to the first amplification product. This allows products to be replicated by a strand-displacement mechanism, which can yield a drastic amplification within an isothermal reaction (Lage et al., Genome Research 13:294-307 (2003), which is incorporated herein by reference in its entirety).

In embodiments, methods for amplification include, but are not limited to, the polymerase chain reaction (PCR), strand displacement amplification (SDA), transcription mediated amplification (TMA) and nucleic acid sequence-based amplification (NASBA), for example, as described in U.S. Pat. No. 8,003,354, which is incorporated herein by reference in its entirety. The above amplification methods can be employed to amplify one or more nucleic acids of interest. For example, PCR, multiplex PCR, SDA, TMA, NASBA and the like can be utilized to amplify immobilized nucleic acid fragments generated from the first amplification method of the two-step method described herein.

In embodiments, the amplifying includes bridge amplification; for example as exemplified by the disclosures of U.S. Pat. Nos. 5,641,658; 7,115,400; 7,790,418; U.S. Patent Publ. No. 2008/0009420, each of which is incorporated herein by reference in its entirety. In general, bridge amplification uses repeated steps of annealing of primers to templates, primer extension, and separation of extended primers from templates. Because the forward and reverse primers are attached to the solid support, the extension products released upon separation from an initial template are also attached to the solid support. Both strands are immobilized on the solid support at the 5′ end, preferably via a covalent attachment. The 3′ end of an amplification product is then permitted to anneal to a nearby reverse primer, forming a “bridge” structure. The reverse primer is then extended to produce a further template molecule that can form another bridge. During bridge PCR, additional chemical additives may be included in the reaction mixture, in which the DNA strands are denatured by flowing a denaturant over the DNA, which chemically denatures complementary strands. This is followed by washing out the denaturant and reintroducing a polymerase in buffer conditions that allow primer annealing and extension.

In embodiments, the amplifying includes thermal bridge polymerase chain reaction (t-bPCR) amplification. In embodiments, the t-bPCR amplification includes incubation in an additive that lowers a DNA denaturation temperature. In embodiments, the additive is betaine, dimethyl sulfoxide (DMSO), ethylene glycol, formamide, glycerol, guanidine thiocyanate, 4-methylmorpholine 4-oxide (NMO), or a mixture thereof. In embodiments, the additive is betaine, DMSO, ethylene glycol, or a mixture thereof. In embodiments, the additive is betaine, DMSO, or ethylene glycol.

In embodiments, the amplifying includes chemical bridge polymerase chain reaction (c-bPCR) amplification. In embodiments, the c-bPCR amplification includes denaturation using a chemical denaturant. In embodiments, the c-bPCR amplification includes denaturation using acetic acid, hydrochloric acid, nitric acid, formamide, guanidine, sodium salicylate, sodium hydroxide, dimethyl sulfoxide (DMSO), propylene glycol, urea, or a mixture thereof. In embodiments, the chemical denaturant is sodium hydroxide or formamide. Chemical bridge polymerase chain reactions include fluidically cycling a denaturant (e.g., formamide) and maintaining the temperature within a narrow temperature range (e.g., +/−5° C.). In contrast, thermal bridge polymerase chain reactions include thermally cycling between high temperatures (e.g., 85° C.-95° C.) and low temperatures (e.g., 60° C.-70° C.). Thermal bridge polymerase chain reactions may also include a denaturant, typically at a significantly lower concentration than traditional chemical bridge polymerase chain reactions.

In embodiments, the amplifying includes fluidic cycling between an extension mixture that includes a polymerase and dNTPs, and a chemical denaturant. In embodiments, the polymerase is a strand-displacing polymerase or a non-strand displacing polymerase. In embodiments, the solutions are thermally cycled between about 40° C. to about 65° C. during fluidic cycling of the extension mixture and the chemical denaturant. For example, the extension cycle is maintained at a temperature of 55° C.-65° C., followed by a denaturation cycle that is maintained at a temperature of 40° C.-65° C., or by a denaturation step in which the temperature starts at 60° C.-65° C. and is ramped down to 40° C. prior to exchanging the reagent. In embodiments, the amplifying includes modulating the reaction temperature prior to initiating the next cycle. In embodiments, the denaturation cycle and/or the extension cycle is maintained at a temperature for a sufficient amount of time, and prior to starting the next cycle the temperature is modulated (e.g., increased relative to the starting temperature or reduced relative to the starting temperature). In embodiments, the denaturation cycle is performed at a temperature of 60° C.-65° C. for about 5-45 sec, then the temperature is reduced (e.g., lowered to about 40° C.) before starting an extension cycle (i.e., before introducing an extension mixture). Lowering the temperature, even in the presence of a chemical denaturant, facilitates primer hybridization in the subsequent step when the amplicons are exposed to conditions that promote hybridization. In embodiments, the extension cycle is performed at a temperature of 50° C.-60° C. for about 0.5-2 minutes, then the temperature is increased (e.g., raised to between about 60° C. to about 70° C., or to about 65° C. to about 72° C.) after introducing the extension mixture. In embodiments, the cycling between the extension mixture and the chemical denaturant is performed at least 5, at least 10, at least 20, at least 30, at least 40, at least 50, at least 75, at least 100, or at least 200 times. In embodiments, the cycling between the extension mixture and the chemical denaturant is performed about 5, about 10, about 20, about 30, about 40, about 50, about 75, about 100, or about 200 times. In embodiments, the cycling between the extension mixture and the chemical denaturant is performed a total of 5, 10, 20, 30, 40, 50, 75, 100, 200, or more times. In embodiments, the fluidic cycling is performed in the presence of about 2 to about 15 mM Mg²⁺. In embodiments, the fluidic cycling is performed in the presence of about 2, about 3, about 4, about 5, about 6, about 7, about 8, about 9, about 10, about 11, about 12, about 13, about 14, or about 15 mM Mg²⁺.

In embodiments, the method further includes aligning a substring of one or more sequencing reads to a reference sequence, and quantifying the number of sequencing reads for the circular template polynucleotide including the fusion junction. In embodiments, the method further includes aligning a substring of one or more sequencing reads to a reference sequence quantifying the number of sequencing reads for the fusion gene circular template polynucleotides, wherein the quantifying includes aligning a substring of the sequencing reads to a reference sequence. In embodiments, the method further includes aligning one or more sequencing reads to a reference sequence.

In embodiments, the method includes comparing k-mer substrings of one or more sequencing reads to a table of k-mers of a fusion gene reference. In embodiments, the method includes quantifying (i.e., measuring and/or detecting) the number of k-mer substrings shared between the sequencing read and the fusion gene reference. In embodiments, the method includes (i) grouping one or more sequencing reads based on a barcode sequence and/or a sequence including the fusion junction; and (ii) within the groups, aligning the reads and forming a consensus sequence for reads having the same barcode sequence and/or sequence including the fusion junction. In embodiments, sequencing further includes generating sequencing reads spanning the circularization junctions formed between 5′ and 3′ ends of the linear nucleic acid molecules, and quantifying the number of different circularization junction sequences (fusion gene circular template polynucleotides) that contain the fusion gene.

In embodiments, the sequencing includes sequencing by synthesis, sequencing-by-binding, sequencing by hybridization, sequencing by ligation, or pyrosequencing. A variety of sequencing methodologies can be used such as sequencing by synthesis (SBS), pyrosequencing, sequencing by ligation (SBL), or sequencing by hybridization (SBH). Pyrosequencing detects the release of inorganic pyrophosphate (PPi) as particular nucleotides are incorporated into a nascent nucleic acid strand (Ronaghi, et al., Analytical Biochemistry 242(1), 84-9 (1996); Ronaghi, Genome Res. 11(1), 3-11 (2001); Ronaghi et al. Science 281(5375), 363 (1998); U.S. Pat. Nos. 6,210,891; 6,258,568; and. 6,274,320, each of which is incorporated herein by reference in its entirety). In pyrosequencing, released PPi can be detected by being converted to adenosine triphosphate (ATP) by ATP sulfurylase, and the level of ATP generated can be detected via light produced by luciferase. In this manner, the sequencing reaction can be monitored via a luminescence detection system. In both SBL and SBH methods, target nucleic acids, and amplicons thereof, that are present at features of an array are subjected to repeated cycles of oligonucleotide delivery and detection. SBL methods, include those described in Shendure et al. Science 309:1728-1732 (2005); U.S. Pat. Nos. 5,599,675; and 5,750,341, each of which is incorporated herein by reference in its entirety; and the SBH methodologies are as described in Bains et al., Journal of Theoretical Biology 135(3), 303-7 (1988); Drmanac et al., Nature Biotechnology 16, 54-58 (1998); Fodor et al., Science 251(4995), 767-773 (1995); and WO 1989/10977, each of which is incorporated herein by reference in its entirety.

In SBS, extension of a nucleic acid primer along a nucleic acid template is monitored to determine the sequence of nucleotides in the template. The underlying chemical process can be catalyzed by a polymerase, wherein fluorescently labeled nucleotides are added to a primer (thereby extending the primer) in a template dependent fashion such that detection of the order and type of nucleotides added to the primer can be used to determine the sequence of the template. A plurality of different nucleic acid fragments that have been attached at different locations of an array can be subjected to an SBS technique under conditions where events occurring for different templates can be distinguished due to their location in the array. In embodiments, the sequencing step includes annealing and extending a sequencing primer to incorporate a detectable label that indicates the identity of a nucleotide in the target polynucleotide, detecting the detectable label, and repeating the extending and detecting of steps. In embodiments, the methods include sequencing one or more bases of a target nucleic acid by extending a sequencing primer hybridized to a target nucleic acid (e.g., an amplification product produced by the amplification methods described herein). In embodiments, the sequencing step may be accomplished by a sequencing-by-synthesis (SBS) process. In embodiments, sequencing includes a sequencing by synthesis process, where individual nucleotides are identified iteratively, as they are polymerized to form a growing complementary strand. In embodiments, nucleotides added to a growing complementary strand include both a label and a reversible chain terminator that prevents further extension, such that the nucleotide may be identified by the label before removing the terminator to add and identify a further nucleotide. Such reversible chain terminators include removable 3′ blocking groups, for example as described in U.S. Pat. Nos. 7,541,444, 7,057,026, and 10,738,072. Once such a modified nucleotide has been incorporated into the growing polynucleotide chain complementary to the region of the template being sequenced, there is no free 3′—OH group available to direct further sequence extension and therefore the polymerase cannot add further nucleotides. Once the identity of the base incorporated into the growing chain has been determined, the 3′ block may be removed to allow addition of the next successive nucleotide. By ordering the products derived using these modified nucleotides it is possible to deduce the DNA sequence of the DNA template. Sequencing can be carried out using any suitable sequencing-by-synthesis (SBS) technique, wherein modified nucleotides are added successively to a free 3′ hydroxyl group, typically initially provided by a sequencing primer, resulting in synthesis of a polynucleotide chain in the 5′ to 3′ direction. In embodiments, sequencing includes detecting a sequence of signals. In embodiments, sequencing includes extension of a sequencing primer with labeled nucleotides. Examples of sequencing include, but are not limited to, sequencing by synthesis (SBS) processes in which reversibly terminated nucleotides carrying fluorescent dyes are incorporated into a growing strand, complementary to the target strand being sequenced. In embodiments, the nucleotides are labeled with up to four unique fluorescent dyes. In embodiments, the nucleotides are labeled with at least two unique fluorescent dyes. In embodiments, the readout is accomplished by epifluorescence imaging. Non-limiting examples of suitable labels are described in U.S. Pat. Nos. 8,178,360, 5,188,934 (4,7-dichlorofluorscein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthene dyes): U.S. Pat. No. 5,688,648 (energy transfer dyes); and the like.

In embodiments, generating a first sequencing read or a second sequencing read includes sequencing-by-binding (see, e.g., U.S. Pat. Pubs. US2017/0022553 and US2019/0048404, each of which is incorporated herein by reference in its entirety). As used herein, “sequencing-by-binding” refers to a sequencing technique wherein specific binding of a polymerase and cognate nucleotide to a primed template nucleic acid molecule (e.g., blocked primed template nucleic acid molecule) is used for identifying the next correct nucleotide to be incorporated into the primer strand of the primed template nucleic acid molecule. The specific binding interaction need not result in chemical incorporation of the nucleotide into the primer. In some embodiments, the specific binding interaction can precede chemical incorporation of the nucleotide into the primer strand or can precede chemical incorporation of an analogous, next correct nucleotide into the primer. Thus, detection of the next correct nucleotide can take place without incorporation of the next correct nucleotide. As used herein, the “next correct nucleotide” (sometimes referred to as the “cognate” nucleotide) is the nucleotide having a base complementary to the base of the next template nucleotide. The next correct nucleotide will hybridize at the 3′-end of a primer to complement the next template nucleotide. The next correct nucleotide can be, but need not necessarily be, capable of being incorporated at the 3′ end of the primer. For example, the next correct nucleotide can be a member of a ternary complex that will complete an incorporation reaction or, alternatively, the next correct nucleotide can be a member of a stabilized ternary complex that does not catalyze an incorporation reaction. A nucleotide having a base that is not complementary to the next template base is referred to as an “incorrect” (or “non-cognate”) nucleotide.

Use of the sequencing method outlined above is a non-limiting example, as essentially any sequencing methodology which relies on successive incorporation of nucleotides into a polynucleotide chain can be used. Suitable alternative techniques include, for example, pyrosequencing methods, FISSEQ (fluorescent in situ sequencing), MPSS (massively parallel signature sequencing), or sequencing by ligation-based methods.

In embodiments, the sequencing includes a plurality of sequencing cycles. In embodiments, a sequencing cycle includes extending a complementary polynucleotide by incorporating a first nucleotide using a polymerase, wherein the polynucleotide is hybridized to a template nucleic acid, detecting the first nucleotide, and identifying the first nucleotide. In embodiments, to begin a sequencing cycle, one or more differently labeled nucleotides and a DNA polymerase can be introduced. Following nucleotide addition, signals produced (e.g., via excitation and emission of a detectable label) can be detected to determine the identity of the incorporated nucleotide (based on the labels on the nucleotides). Reagents can then be added to remove the 3′ reversible terminator and to remove label(s) from each incorporated base. Reagents, enzymes and other substances can be removed between steps by washing. Cycles may include repeating these steps, and the sequence of each cluster is read over the multiple repetitions. In embodiments, the sequencing yields reads of greater than 25 bp read length. In embodiments, the sequencing yields reads of greater than 50 bp read length. In embodiments, the sequencing yields reads of greater than 75 bp read length. In embodiments, the sequencing yields reads of greater than 100 bp read length. In embodiments, the sequencing yields reads of greater than 150 bp read length. In embodiments, generating a sequencing read includes determining the identity of the nucleotides in the template polynucleotide.

In embodiments, the sequencing method relies on the use of modified nucleotides that can act as reversible terminators. Once the modified nucleotide has been incorporated into the growing polynucleotide chain complementary to the region of the template being sequenced there is no free 3′—OH group available to direct further sequence extension and therefore the polymerase cannot add further nucleotides. Once the identity of the base incorporated into the growing chain has been determined, the 3′ reversible terminator may be removed to allow addition of the next successive nucleotide. These such reactions can be done in a single experiment if each of the modified nucleotides has attached a different label, known to correspond to the particular base, to facilitate discrimination between the bases added at each incorporation step. Alternatively, a separate reaction may be carried out containing each of the modified nucleotides separately.

The modified nucleotides may carry a label (e.g., a fluorescent label) to facilitate their detection. Each nucleotide type may carry a different fluorescent label. However, the detectable label need not be a fluorescent label. Any label can be used which allows the detection of an incorporated nucleotide. One method for detecting fluorescently labeled nucleotides includes using laser light of a wavelength specific for the labeled nucleotides, or the use of other suitable sources of illumination. The fluorescence from the label on the nucleotide may be detected (e.g., by a CCD camera or other suitable detection means).

In embodiments, the methods of sequencing a nucleic acid include extending a complementary polynucleotide (e.g., a primer) that is hybridized to the nucleic acid by incorporating a first nucleotide (e.g., a modified, labeled nucleotide). In embodiments, the method includes a buffer exchange or wash step. In embodiments, the methods of sequencing a nucleic acid include a sequencing solution. The sequencing solution includes (a) an adenine nucleotide, or analog thereof; (b) (i) a thymine nucleotide, or analog thereof, or (ii) a uracil nucleotide, or analog thereof; (c) a cytosine nucleotide, or analog thereof and (d) a guanine nucleotide, or analog thereof.

In embodiments, the sequencing includes extending a sequencing primer by incorporating a labeled nucleotide, or labeled nucleotide analogue, and detecting the label to generate a signal for each incorporated nucleotide or nucleotide analogue, wherein the sequencing primer is hybridized to one of the fusion amplification products.

In embodiments, detecting the fusion amplification products includes aligning a sub string of each sequencing read to a reference sequence, and quantifying the number of aligned sequencing reads for the fusion gene circular template polynucleotides.

In embodiments, detecting the fusion amplification products includes comparing k-mer substrings of each sequencing read to a table of k-mers of a fusion junction reference, and quantifying the number of k-mers shared between the sequencing read and the fusion junction reference. The term “fusion junction reference” refers to a collection of sequences of previously detected fusions involving the one or more genes of interest.

In embodiments, detecting the fusion amplification products includes (i) grouping sequencing reads based on a barcode sequence and/or a sequence including the fusion junction; and (ii) within each group, aligning the reads and forming a consensus sequence for reads having the same barcode sequence and/or sequence including the fusion junction.

In embodiments, the sequencing further includes generating sequencing reads including the circularization junctions formed between 5′ and 3′ ends of the linear nucleic acid molecules and quantifying the number of different circularization junction sequences that contain the fusion junction. In embodiments, the sequencing further includes generating sequencing reads that includes the circularization junction formed between the 5′ and 3′ ends of the linear nucleic acid molecules, and quantifying the number of different circularization junction sequences that contain the fusion junction.

In embodiments, the method further includes quantifying the fusion amplification products. Molecular counting of fusion amplification products is useful for diagnostic purposes. As described herein, the polynucleotides containing fusions are preferentially amplified enabling precise quantification over large background levels. Conventional bioinformatic analyses may be used to quantify fusion amplification products. In some embodiments, bioinformatic analyses may involve counting the number of unique circularization junctions associated with a particular fusion amplification product. In other embodiments, quantification of fusion amplification products is accomplished by comparing the number of sequencing reads or circularization junctions corresponding to the fusion amplification products to those for a control (e.g., spike in control) present at a predetermined number of template copies. In yet other embodiments, quantification may be performed by qPCR or semiquantitative PCR.

In embodiments, the one or more linear nucleic acid molecules are derived from a sample of a subject, optionally wherein the sample is an FFPE sample. In embodiments, the FFPE sample is incubated with xylene and washed using ethanol to remove the embedding wax, followed by treatment with Proteinase K to permeabilized the tissue. In embodiments, the one or more linear nucleic acid molecules are derived from a liquid biopsy (e.g., plasma). In embodiments, the one or more linear nucleic acid molecules are derived from the sample, which is frozen tissue, formalin-fixed paraffin-embedded (FFPE) tissue, peripheral blood, bone marrow, or cerebral spinal fluid.

In embodiments, the polynucleotide fusion is a biomarker for a cancer, an autoimmune disease, a primary immunodeficiency, or an infectious disease. In embodiments, the polynucleotide fusion is a biomarker for a cancer. In embodiments, the polynucleotide fusion is a biomarker for a lymphoid malignancy. In embodiments, the polynucleotide fusion is a biomarker for a primary immunodeficiency. In embodiments, the polynucleotide fusion is a biomarker for an infectious disease. A “biomarker” is a substance that is associated with a particular characteristic, such as a disease or condition. A change in the levels of a biomarker may correlate with the risk or progression of a disease or with the susceptibility of the disease to a given treatment.

In embodiments, the fusion gene causes a disease in a subject in which the fusion gene is found. In embodiments, the fusion gene is associated with a disease. In embodiments, the disease is cancer, an autoimmune disease, a primary immunodeficiency, or an infectious disease. In some embodiments, the disease is an infectious disease, an autoimmune disease, hereditary disease, or cancer. In embodiments, the disease is an acute disease, a chronic disease (e.g., a malady that exists for greater than 6 months), an idiopathic disease, or a syndrome (e.g., Down syndrome). In embodiments, the disease is a relapsed disease (e.g., a malady that is detectable after a period of time of not being detectable).

In embodiments, the amplification reaction further includes: (a) one or more different first primers that specifically hybridize to different portions of the first strand of the first region; (b) for each different first primer, a different second primer that specifically hybridizes to a complement of a portion of the first strand of the first region that is 3′ with respect to where the corresponding different first primer specifically hybridizes; and (c) for each different first primer, a different blocking oligo that specifically hybridizes to a portion of the first strand of the first region that is 5′ with respect to where the different first primer specifically hybridizes.

In embodiments, the fusion gene includes a sequence encoding for a B cell receptor. In embodiments, the fusion gene includes a sequence encoding for a T cell receptor.

In embodiments, the fusion gene includes an IGH locus or a BCL-1, BCL-2, BCL-3, or BCL6 locus.

In embodiments, the fusion gene includes a sequence encoding for a complementarity-determining region (CDR) of a T cell receptor or a B cell receptor. In embodiments, the fusion gene includes a sequence encoding for the CDR3 region of a T cell receptor or a B cell receptor.

In embodiments, the fusion gene includes a sequence encoding for a V region or a complement thereof and a J region or a complement thereof.

In embodiments, the polynucleotide fusion includes a gene, or a portion thereof, encoding a kinase domain. In embodiments, the polynucleotide fusion includes a gene fusion of BCL1-JH, BCL2-JH, or MYC-IGL.

In embodiments, the polynucleotide fusion includes a B-cell or T-Cell intrachromosomal rearrangement. In embodiments, the polynucleotide fusion includes a B-cell intrachromosomal rearrangement. In embodiments, the polynucleotide fusion includes a T-cell intrachromosomal rearrangement.

In embodiments, the polynucleotide fusion includes a fusion of a rearranged T cell antigen receptor or fragment thereof, a T cell receptor alpha variable (TRAV) gene or fragment thereof, a T cell receptor alpha joining (TRAJ) gene or fragment thereof, a T cell receptor alpha constant (TRAC) gene or fragment thereof, a T cell receptor beta variable (TRBV) gene or fragment thereof, a T cell receptor beta diversity (TRBD) gene or fragment thereof, a T cell receptor beta joining (TRBJ) gene or fragment thereof, a T cell receptor beta constant (TRBC) gene or fragment thereof, a T cell receptor gamma variable (TRGV) gene or fragment thereof, a T cell receptor gamma joining (TRGJ) gene or fragment thereof, a T cell receptor gamma constant (TRGC) gene or fragment thereof, a T cell receptor delta variable (TRDV) gene or fragment thereof, a T cell receptor delta diversity (TRDD) gene or fragment thereof, a T cell receptor delta joining (TRDJ) gene or fragment thereof, or a T cell receptor delta constant (TRDC) gene or fragment thereof.

In embodiments, the polynucleotide fusion includes a fusion of a rearranged B cell antigen receptor or fragment thereof, an IGHV gene or fragment thereof, an IGHD gene or fragment thereof, or an IGHJ gene or fragment thereof, IGHJC gene or fragment thereof, an IGKV gene or fragment thereof, an IGKJ gene or fragment thereof, an IGKC gene or fragment thereof, an IGLV gene or portion thereof, an IGLJ gene or portion thereof, an IGLC gene or fragment thereof, an IGK kappa deletion element or portion thereof, a IGK intronic enhancer element or portion thereof. In embodiments, the polynucleotide fusion includes a fusion of an ALK gene or portion thereof, a BRAF gene or portion thereof, an EGFR gene or portion thereof, an ERBB2 gene or portion thereof, a KRAS gene or portion thereof, a MET gene or portion thereof, an NRG1 gene or portion thereof, an FGFR1 gene or portion thereof, an FGFR2 gene or portion thereof, an FGFR3 gene or portion thereof, an NTRK1 gene or portion thereof, an NTRK2 gene or portion thereof, an NTRK3 gene or portion thereof, a RET gene or portion thereof, or a ROS1 gene or portion thereof.

In embodiments, the fusion gene includes a RBPSM-MET, BCAN-NTRK1, TRIM22-BRAF, KIAA1549-BRAF, FGFR1-TACC1, EWSR1-FLI1, PAX3-FOXO1, ZFTA-RELA, COL3A1-PLAG1, FGFR3-TACC3, or NPM1-ALK fusion gene.

In embodiments, the cancer is a lymphoid hematological malignancy, wherein the lymphoid hematological malignancy is acute T-cell lymphoblastic leukemia (T-ALL), acute B-cell lymphoblastic leukemia (B-ALL), multiple myeloma, plasmacytoma, macroglobulinemia, chronic lymphocytic leukemia (CLL), acute lymphoblastic leukemia (ALL), Hodgkins lymphoma, non-Hodgkins lymphoma, cutaneous T-cell lymphoma, mantle cell lymphoma, peripheral T-cell lymphoma, hairy cell leukemia, T prolymphocytic lymphoma, angioimmunoblastic T-cell lymphoma, T lymphoblastic leukemia/lymphoma, peripheral T-cell lymphoma, adult T cell leukemia/lymphoma, mycosis fungoides, Sezary syndrome, T lymphoblastic leukemia, myeloproliferative neoplasm, and myelodysplastic syndrome.

In embodiments, the fusion gene is associated with a pediatric cancer. In embodiments, the fusion gene is associated with an adult cancer. In embodiments, the fusion gene is associated with cancer in an animal (e.g., a mammal).

In embodiments, the cancer is a pilocytic astrocytoma, Ewing sarcoma, supratentorial ependymoma, infantile fibrosarcoma, cholangiocarcinoma, infantile spindle cell sarcoma, infiltrating glioma, ganglioglioma, or acute lymphocytic leukemia.

In embodiments, the sample includes at least 100,000 T cells or B cells. In embodiments, the sample includes at least 90,000 T cells or B cells. In embodiments, the sample includes at least 80,000 T cells or B cells. In embodiments, the sample includes at least 70,000 T cells or B cells. In embodiments, the sample includes at least 60,000 T cells or B cells. In embodiments, the sample includes at least 50,000 T cells or B cells. In embodiments, the sample includes at least 40,000 T cells or B cells. In embodiments, the sample includes at least 30,000 T cells or B cells. In embodiments, the sample includes at least 20,000 T cells or B cells. In embodiments, the sample includes at least 10,000 T cells or B cells.

In embodiments, the sample is fresh tissue, frozen tissue, formalin-fixed paraffin-embedded (FFPE) tissue, peripheral blood, bone marrow, or cerebral spinal fluid. In embodiments, the sample includes cells and/or nuclei that are not present in an aggregated form or clump. In embodiments, the sample is a tumor tissue sample. Tumor samples may contain only tumor cells, or they may contain both tumor cells and non-tumor cells. In particular embodiments, a tissue section includes only non-tumor cells. In particular embodiments, the tumor is a solid tumor.

In embodiments, the sample is obtained pre-treatment or post-treatment, wherein treatment includes a therapeutic intervention (e.g., a pharmaceutical treatment, radiation treatment, chemotherapy, surgery, etc.). In some embodiments, the sample is obtained pre-transplant or post-transplant. In embodiments, the sample is obtained at a plurality of timepoints post-treatment. In embodiments, the sample is obtained at a plurality of timepoints pre-treatment. In embodiments, a plurality of samples is obtained at two or more time points. In embodiments, a plurality of samples is obtained at 3 or more time points. In embodiments, a plurality of samples is obtained at 4 or more time points. In embodiments, a plurality of samples is obtained at 5 or more time points. In embodiments, a plurality of samples are obtained at two or more serial time points, wherein each time point is at a fixed interval (e.g., 30 minutes, 1 hour, 2 hours, 4 hours, 6 hours, 12 hours, 1 day, 7 days, 2 weeks, 3 weeks, 1 month, 2 months, 3 months, 6 months, 9 months, 1 year, 2 years, 3 years, or more). In embodiments, a plurality of samples is obtained at one or more time points pre-treatment and at one or more time points post-treatment.

Tissue sections may be obtained from a subject by any means known and available in the art. In particular embodiments, a tissue section, e.g., a tumor tissue sample, is obtained from a subject by fine needle aspiration, core needle biopsy, stereotactic core needle biopsy, vacuum-assisted core biopsy, or surgical biopsy. In particular embodiments, the surgical biopsy is an incisional biopsy, which removes only part of the suspicious area. In other embodiments, the surgical biopsy is an excisional biopsy, which removes the entire diseased tissue (e.g., tumor) or abnormal area. In particular embodiments, an excisional tumor tissue sample is obtained from a tumor that has been excised with the intent to “cure” a patient in the case of early stage disease, wherein in other embodiments, the excisional tumor tissue sample is obtained from an excised bulk of primary tumor in later stage disease. Tumor tissue samples may include primary tumor tissue, metastatic tumor tissue and/or secondary tumor tissue. Tumor tissue samples may be cell cultures, e.g., cultures of tumor-derived cell lines. In certain embodiments, a tissue section is a cell line, e.g., a cell pellet of a cultured cell line, such as a tumor cell line. In particular embodiments, the cell line or cell pellet is frozen or was previously frozen. Such cell lines and pellets are useful, e.g., as positive or negative controls for imaging with various reagents. Tumor tissue samples may also be xenograft tumors, e.g., tumors obtained from animals administered with tumor cells, e.g., a human tumor cell line. In certain embodiments, a first tumor tissue sample from a subject is a primary tumor tissue sample obtained during an initial surgery intended to remove the entire tumor, and a second tumor tissue sample is obtained from the same subject is a metastatic tumor tissue sample or a secondary tumor tissue sample obtained during a later surgery.

In embodiments, the sample is obtained from a subject (e.g., human or animal tissue). Once obtained, the sample is placed in an artificial environment in plastic or glass containers supported with specialized medium containing essential nutrients and growth factors to support proliferation. In embodiments, the sample is permeabilized and immobilized to a solid support surface. In embodiments, the sample is permeabilized and immobilized to an array (i.e., to discrete locations arranged in an array). In embodiments, the sample is immobilized to a solid support surface. In embodiments, the surface includes a patterned surface (e.g., suitable for immobilization of a plurality of cells in an ordered pattern. The discrete regions of the ordered pattern may have defined locations in a regular array, which may correspond to a rectilinear pattern, circular pattern, hexagonal pattern, or the like. These discrete regions are separated by interstitial regions. As used herein, the term “interstitial region” refers to an area in a substrate or on a surface that separates other areas of the substrate or surface. In embodiments, a plurality of cells is immobilized on a patterned surface that have a mean or median separation from one another of about 10-20 μm. In embodiments, a plurality of cells is immobilized on a patterned surface that have a mean or median separation from one another of about 10-20; 10-50; or 100 μm. In embodiments, a plurality of cells is arrayed on a substrate. In embodiments, a plurality of cells is immobilized in a 96-well microplate having a mean or median well-to-well spacing of about 8 mm to about 12 mm (e.g., about 9 mm). In embodiments, a plurality of cells is immobilized in a 384-well microplate having a mean or median well-to-well spacing of about 3 mm to about 6 mm (e.g., about 4.5 mm).

In embodiments, the sample (e.g., the tissue section or plurality of cells) is attached to the receiving substrate via a bioconjugate reactive linker. In embodiments, the tissue section is attached to the substrate via a specific binding reagent. In embodiments, the specific binding reagent includes an antibody, single-chain Fv fragment (scFv), antibody fragment-antigen binding (Fab), or an aptamer. In embodiments, the specific binding reagent includes an antibody, or antigen binding fragment, an aptamer, affimer, or non-immunoglobulin scaffold. In embodiments, the specific binding reagent is a peptide, a cell penetrating peptide, an aptamer, a DNA aptamer, an RNA aptamer, an antibody, an antibody fragment, a light chain antibody fragment, a single-chain variable fragment (scFv), a lipid, a lipid derivative, a phospholipid, a fatty acid, a triglyceride, a glycerolipid, a glycerophospholipid, a sphingolipid, a saccharolipid, a polyketide, a polylysine, polyethyleneimine, diethylaminoethyl (DEAE)-dextran, cholesterol, or a sterol moiety. Substrates may be prepared for selective capture of particular cells of the tissue section. For example, a substrate containing a plurality of bioconjugate reactive moieties or a plurality of specific binding reagents, optionally in an ordered pattern, contacts a plurality of cells of the tissue section. Only cells of the tissue section containing complementary bioconjugate reactive moieties or complementary specific binding reagents are capable of reacting, and thus adhering, to the substrate.

In embodiments, the methods are performed in situ in tissue sections that have been prepared according to methodologies known in the art. Methods for permeabilization and fixation of cells and tissue samples are known in the art, as exemplified by Cremer et al., The Nucleus: Volume 1: Nuclei and Subnuclear Components, R. Hancock (ed.) 2008; and Larsson et al., Nat. Methods (2010) 7:395-397, the content of each of which is incorporated herein by reference in its entirety. In embodiments, the tissue section is cleared (e.g., digested) of proteins, lipids, or proteins and lipids.

In embodiments, the tissue section forms part of a tissue in situ. In embodiments, the tissue section includes one or more prokaryotic cells. In embodiments, the tissue section includes one or more eukaryotic cells. In embodiments, the tissue section includes a bacterial cell (e.g., a bacterial cell or bacterial spore), a fungal cell (e.g., a fungal spore), a plant cell, or a mammalian cell. In embodiments, the tissue section includes a stem cell. In embodiments, the stem cell is an embryonic stem cell, a tissue-specific stem cell, a mesenchymal stem cell, or an induced pluripotent stem cell. In embodiments, the tissue section includes an endothelial cell, muscle cell, myocardial, smooth muscle cell, skeletal muscle cell, mesenchymal cell, epithelial cell; hematopoietic cell, such as lymphocytes, including T cell, e.g., (Th1 T cell, Th2 T cell, ThO T cell, cytotoxic T cell); B cell, pre-B cell; monocytes; dendritic cell; neutrophils; or a macrophage. In embodiments, the tissue section includes a stem cell, an immune cell, a cancer cell (e.g., a circulating tumor cell or cancer stem cell), a viral-host cell, or a cell that selectively binds to a desired target. In embodiments, the cell includes a T cell receptor gene sequence, a B cell receptor gene sequence, or an immunoglobulin gene sequence. In embodiments, the cell includes a Toll-like receptor (TLR) gene sequence. In embodiments, the cell includes a gene sequence corresponding to an immunoglobulin light chain polypeptide and a gene sequence corresponding to an immunoglobulin heavy chain polypeptide. In embodiments, the tissue section includes a genetically modified cell. In embodiments, the tissue section includes a circulating tumor cell or cancer stem cell.

In embodiments, the tissue section includes an adherent cell (e.g., epithelial cell, endothelial cell, or neural cell). Adherent cells are usually derived from tissues of organs and attach to a substrate (e.g., epithelial cells adhere to an extracellular matrix coated substrate via transmembrane adhesion protein complexes). Adherent cells typically require a substrate, e.g., tissue culture plastic, which may be coated with extracellular matrix (e.g., collagen and laminin) components to increase adhesion properties and provide other signals needed for growth and differentiation. In embodiments, the tissue section includes a neuronal cell, an endothelial cell, epithelial cell, germ cell, plasma cell, a muscle cell, peripheral blood mononuclear cell (PBMC), a myocardial cell, or a retina cell. In embodiments, the tissue section includes a suspension cell (e.g., a cell free-floating in the culture medium, such a lymphoblast or hepatocyte). In embodiments, the tissue section includes a glial cell (e.g., astrocyte, radial glia), pericyte, or stem cell (e.g., a neural stem cell). In embodiments, the tissue section includes a neuronal cell. In embodiments, the tissue section includes an endothelial cell. In embodiments, the tissue section includes an epithelial cell. In embodiments, the tissue section includes a germ cell. In embodiments, the tissue section includes a plasma cell. In embodiments, the tissue section includes a muscle cell. In embodiments, the tissue section includes a peripheral blood mononuclear cell (PBMC). In embodiments, the tissue section includes a myocardial cell. In embodiments, the tissue section includes a retina cell. In embodiments, the tissue section includes a lymphoblast. In embodiments, the tissue section includes a hepatocyte. In embodiments, the tissue section includes a glial cell. In embodiments, the tissue section includes an astrocyte. In embodiments, the tissue section includes a radial glia. In embodiments, the tissue section includes a pericyte. In embodiments, the tissue section includes a stem cell. In embodiments, the tissue section includes a neural stem cell.

In embodiments, the tissue section includes a cell bound to a known antigen. In embodiments, the cell is a cell that selectively binds to a desired target, wherein the target is an antibody, or antigen binding fragment, an aptamer, affimer, non-immunoglobulin scaffold, small molecule, or genetic modifying agent. In embodiments, the cell is a leukocyte (i.e., a white-blood cell). In embodiments, leukocyte is a granulocyte (neutrophil, eosinophil, or basophil), monocyte, or lymphocyte (T cells and B cells). In embodiments, the cell is a lymphocyte. In embodiments, the cell is a T cell, an NK cell, or a B cell.

In embodiments, the tissue section includes an immune cell. In embodiments, the immune cell is a granulocyte, a mast cell, a monocyte, a neutrophil, a dendritic cell, or a natural killer (NK) cell. In embodiments, the immune cell is an adaptive cell, such as a T cell, NK cell, or a B cell. In embodiments, the cell includes a T cell receptor gene sequence, a B cell receptor gene sequence, or an immunoglobulin gene sequence. In embodiments, the immune cell is a granulocyte. In embodiments, the immune cell is a mast cell. In embodiments, the immune cell is a monocyte. In embodiments, the immune cell is a neutrophil. In embodiments, the immune cell is a dendritic cell. In embodiments, the immune cell is a natural killer (NK) cell. In embodiments, the immune cell is a T cell. In embodiments, the immune cell is a B cell. In embodiments, the cell includes a T cell receptor gene sequence. In embodiments, the cell includes a B cell receptor gene sequence. In embodiments, the cell includes an immunoglobulin gene sequence. In embodiments, the plurality of target nucleic acids includes non-contiguous regions of a nucleic acid molecule. In embodiments, the non-contiguous regions include regions of a VDJ recombination of a B cell or T cell.

In embodiments, the infectious disease is a disease or disorder associated with an infection from a pathogenic organism. In embodiments, the infectious disease is Acinetobacter infections, Actinomycosis, African sleeping sickness (African trypanosomiasis), AIDS (acquired immunodeficiency syndrome), Amoebiasis, Anaplasmosis, Angiostrongyliasis, Anisakiasis, Anthrax, Arcanobacterium haemolyticum infection, Argentine hemorrhagic fever, Ascariasis, Aspergillosis, Astrovirus infection, Babesiosis, Bacillus cereus infection, Bacterial meningitis, Bacterial pneumonia, Bacterial vaginosis, Bacteroides infection, Balantidiasis, Bartonellosis, Baylisascaris infection, BK virus infection, Black piedra, Blastocystosis, Blastomycosis, Bolivian hemorrhagic fever, Botulism (and Infant botulism), Brazilian hemorrhagic fever, Brucellosis, Bubonic plague, Burkholderia infection, Buruli ulcer, Calicivirus infection (Norovirus and Sapovirus), Campylobacteriosis, Candidiasis (Moniliasis; Thrush), Capillariasis, Carrion's disease, Cat-scratch disease, Cellulitis, Chagas disease (American trypanosomiasis), Chancroid, Chickenpox, Chikungunya, Chlamydia, Chlamydophila pneumoniae infection (Taiwan acute respiratory agent or TWAR), Cholera, Chromoblastomycosis, Chytridiomycosis, Clonorchiasis, Clostridium difficile colitis, Coccidioidomycosis, Colorado tick fever (CTF), Common cold (Acute viral rhinopharyngitis; Acute coryza), Coronavirus disease 2019 (COVID-19), Creutzfeldt-Jakob disease (CJD), Crimean-Congo hemorrhagic fever (CCHF), Cryptococcosis, Cryptosporidiosis, Cutaneous larva migrans (CLM), Cyclosporiasis, Cysticercosis, Cytomegalovirus infection, Dengue fever, Desmodesmus infection, Dientamoebiasis, Diphtheria, Diphyllobothriasis, Dracunculiasis, Ebola hemorrhagic fever, Echinococcosis, Ehrlichiosis, Enterobiasis (Pinworm infection), Enterococcus infection, Enterovirus infection, Epidemic typhus, Erythema infectiosum (Fifth disease), Exanthem subitum (Sixth disease), Fasciolasis, Fasciolopsiasis, Fatal familial insomnia (FFI), Filariasis, Food poisoning by Clostridium perfringens, Free-living amebic infection, Fusobacterium infection, Gas gangrene (Clostridial myonecrosis), Geotrichosis, Gerstmann-Sträussler-Scheinker syndrome (GSS), Giardiasis, Glanders, Gnathostomiasis, Gonorrhea, Granuloma inguinale (Donovanosis), Group A streptococcal infection, Group B streptococcal infection, Haemophilus influenzae infection, Hand, foot and mouth disease (HFMD), Hantavirus Pulmonary Syndrome (HPS), Heartland virus disease, Helicobacter pylori infection, Hemolytic-uremic syndrome (HUS), Hemorrhagic fever with renal syndrome (HFRS), Hendra virus infection, Hepatitis A, Hepatitis B, Hepatitis C, Hepatitis D, Hepatitis E, Herpes simplex, Histoplasmosis, Hookworm infection, Human bocavirus infection, Human ewingii ehrlichiosis, Human granulocytic anaplasmosis (HGA), Human metapneumovirus infection, Human monocytic ehrlichiosis, Human papillomavirus (HPV) infection, Human parainfluenza virus infection, Hymenolepiasis, Epstein-Barr virus infectious mononucleosis (Mono), Influenza (flu), Isosporiasis, Kawasaki disease, Keratitis, Kingella kingae infection, Kuru, Lassa fever, Legionellosis (Legionnaires' disease), Pontiac fever, Leishmaniasis, Leprosy, Leptospirosis, Listeriosis, Lyme disease (Lyme borreliosis), Lymphatic filariasis (Elephantiasis), Lymphocytic choriomeningitis, Malaria, Marburg hemorrhagic fever (MHF), Measles, Middle East respiratory syndrome (MERS), Melioidosis (Whitmore's disease), Meningitis, Meningococcal disease, Metagonimiasis, Microsporidiosis, Molluscum contagiosum (MC), Monkeypox, Mumps, Murine typhus (Endemic typhus), Mycoplasma pneumonia, Mycoplasma genitalium infection, Mycetoma, Myiasis, Neonatal conjunctivitis (Ophthalmia neonatorum), Nipah virus infection, Norovirus, Variant Creutzfeldt-Jakob disease (vCJD, nvCJD), Nocardiosis, Onchocerciasis (River blindness), Opisthorchiasis, Paracoccidioidomycosis (South American blastomycosis), Paragonimiasis, Pasteurellosis, Pediculosis capitis (Head lice), Pediculosis corporis (Body lice), Pediculosis pubis (pubic lice, crab lice), Pelvic inflammatory disease (PID), Pertussis (whooping cough), Plague, Pneumococcal infection, Pneumocystis pneumonia (PCP), Pneumonia, Poliomyelitis, Prevotella infection, Primary amoebic meningoencephalitis (PAM), Progressive multifocal leukoencephalopathy, Psittacosis, Q fever, Rabies, Relapsing fever, Respiratory syncytial virus infection, Rhinosporidiosis, Rhinovirus infection, Rickettsial infection, Rickettsialpox, Rift Valley fever (RVF), Rocky Mountain spotted fever (RMSF), Rotavirus infection, Rubella, Salmonellosis, Severe acute respiratory syndrome (SARS), Scabies, Scarlet fever, Schistosomiasis, Sepsis, Shigellosis (bacillary dysentery), Shingles (Herpes zoster), Smallpox (variola), Sporotrichosis, Staphylococcal food poisoning, Staphylococcal infection, Strongyloidiasis, Subacute sclerosing panencephalitis, Bejel, Syphilis, and Yaws, Taeniasis, Tetanus (lockjaw), Tinea barbae (barber's itch), Tinea capitis (ringworm of the scalp), Tinea corporis (ringworm of the body), Tinea cruris (Jock itch), Tinea manum (ringworm of the hand), Tinea nigra, Tinea pedis (athlete's foot), Tinea unguium (onychomycosis), Tinea versicolor (Pityriasis versicolor), Toxic shock syndrome (TSS), Toxocariasis (ocular larva migrans (OLM)), Toxocariasis (visceral larva migrans (VLM)), Toxoplasmosis, Trachoma, Trichinosis, Trichomoniasis, Trichuriasis (whipworm infection), Tuberculosis, Tularemia, Typhoid fever, Typhus fever, Ureaplasma urealyticum infection, Valley fever, Venezuelan equine encephalitis, Venezuelan hemorrhagic fever, Vibrio vulnificus infection, Vibrio parahaemolyticus enteritis, Viral pneumonia, West Nile fever, White piedra (tinea blanca), Yersinia pseudotuberculosis infection, Yersiniosis, Yellow fever, Zeaspora, Zika fever, or Zygomycosis.

In embodiments, the disease is an autoimmune disease. In embodiments, the autoimmune disease is arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, ichthyosis, Graves ophthalmopathy, inflammatory bowel disease, Addison's disease, Vitiligo, asthma, allergic asthma, acne vulgaris, celiac disease, chronic prostatitis, inflammatory bowel disease, pelvic inflammatory disease, reperfusion injury, ischemia reperfusion injury, stroke, sarcoidosis, transplant rejection, interstitial cystitis, atherosclerosis, scleroderma, or atopic dermatitis. In embodiments, the autoimmune disease is Achalasia, Addison's disease, Adult Still's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Baló disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Thyroid eye disease (TED), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, or Vogt-Koyanagi-Harada Disease.

In embodiments the disease is a hereditary disease. In embodiments, the hereditary disease is cystic fibrosis, alpha-thalassemia, beta-thalassemia, sickle cell anemia (sickle cell disease), Marfan syndrome, fragile X syndrome, Huntington's disease, or hemochromatosis.

In embodiments, the polynucleotide fusion includes a sequence of a first region fused to a sequence of a second region at a fusion junction, wherein the fusion is between two gene sequences, referred to as a gene fusion. The fusion junction may represent the location where the first nucleotide sequence (e.g., a first gene sequence or gene fragment) meets, or is connected to the second nucleotide sequence (e.g., a second gene or gene fragment). In embodiments, a polynucleotide fusion is a hybrid gene formed from two previously independent genes (or gene fragments). In some embodiments, the fusion junction is located between the sequence that specifically is bound by to the blocking element and the sequence that specifically hybridizes to the first primer. In embodiments, the polynucleotide fusion includes a gene fusion of AGTRAP-BRAF, AKAP9-BRAF, ATIC-ALK, CCDC6-RET, CD74-NRG1, CD74-ROS1, CEP89-BRAF, CLCN6-BRAF, DCTN1-ALK, EML4-ALK, EZR-ROS1, FAM131B-BRAF, FCHSD1-BRAF, GATM-BRAF, GNAI1-BRAF, GOLGA5-RET, GOPC-ROS1, HIP1-ALK, HOOK3-RET, KIF5B-ALK, KIF5B-RET, KTN1-RET, LRIG3-ROS1, LSM14A-BRAF, MKRN1-BRAF, MSN-ALK, MYO5A-ROS1, NCOA4-RET, PCM1-RET, RANBP2-ALK, RELCH-RET, RNF130-BRAF, SDC4-ROS1, SLC34A2-ROS1, SLC3A2-NRG1, SLC45A3-BRAF, SQSTM1-ALK, STRN-ALK, TFG-ALK, TPM3-ROS1, TPR-ALK, TRIM24-BRAF, TRIM24-RET, TRIM27-RET, TRIM33-RET, VCL-ALK, WDCP-ALK, ZCCHC8-ROS1, or gene fragments of any of the foregoing fusions.

In embodiments, the polynucleotide fusion includes a gene fusion of ACSL3-ETV1, ACTB-GLI1, AGPAT5-MCPH1, AGTRAP-BRAF, AKAP9-BRAF, ARID1A-MAST2, ATIC-ALK, BBS9-PKD1L1, BCR-JAK2, CBFA2T3-GLIS2, CCDC6-RET, CD74-NRG1, CD74-ROS1, CENPK-KMT2A, CEP89-BRAF, CLCN6-BRAF, COL1A1-PDGFB, COL1A2-PLAG1, CRTC3-MAML2, DCTN1-ALK, DDX5-ETV4, DHH-RHEBL1, DNAJB1-PRKACA, EIF3E-RSPO2, EIF3K-CYP39A1, EML4-ALK, EPC1-PHF1, ETV6-ITPR2, ETV6-JAK2, ETV6-PDGFRB, ETV6-RUNX1, EZR-ERBB4, EZR-ROS1, FAM131B-BRAF, FBXL18-RNF216, FCHSD1-BRAF, FUS-ATF1, FUS-CREB3L1, FUS-CREB3L2, FUS-FEV, GATM-BRAF, GMDS-PDE8B, GNAI1-BRAF, GOLGA5-RET, GOPC-ROS1, HACL1-RAF 1, HAS2-PLAG1, HIP1-ALK, HOOK3-RET, IL6R-ATP8B2, INTS4-GAB2, IRF2BP2-CDX1, JAZF1-PHF1, JAZF1-SUZ12, JPT1-USH1G, KIF5B-ALK, KIF5B-RET, KLK2-ETV1, KLK2-ETV4, KMT2A-ABI1, KMT2A-ACTN4, KMT2A-AFF3, KMT2A-AFF4, KMT2A-ARHGAP26, KMT2A-ARHGEF12, KMT2A-BTBD18, KMT2A-CASP8AP2, KMT2A-CBL, KMT2A-CEP170B, KMT2A-CIP2A, KMT2A-CREBBP, KMT2A-EEFSEC, KMT2A-ELL, KMT2A-EP300, KMT2A-EPS15, KMT2A-FOXO4, KMT2A-FRYL, KMT2A-GAS7, KMT2A-GMPS, KMT2A-GPHN, KMT2A-KNL1, KMT2A-LASP1, KMT2A-LPP, KMT2A-MAPRE1, KMT2A-MLLT1, KMT2A-MLLT11, KMT2A-MLLT3, KMT2A-MLLT6, KMT2A-MYO1F, KMT2A-NCKIPSD, KMT2A-NRIP3, KMT2A-PDS5A, KMT2A-PICALM, KMT2A-SARNP, KMT2A-SH3GL1, KMT2A-TET1, KMT2A-ZFYVE19, KTN1-RET, LIFR-PLAG1, LRIG3-ROS1, LSM14A-BRAF, MBOAT2-PRKCE, MBTD1-CXorf67, MEAF6-PHF1, MKRN1-BRAF, MN1-ETV6, MSN-ALK, MYO5A-ROS1, NAB2-STAT6, NCOA4-RET, NF1-ASIC2, NONO-TFE3, NOTCH1-GABBR2, NTN1-ACLY, NUP107-LGR5, NUP98-KDM5A, PAX3-FOXO1, PAX3-NCOA1, PAX3-NCOA2, PAX5-JAK2, PAX7-FOXO1, PCM1-JAK2, PCM1-RET, PLA2R1-RBMS1, PLXND1-TMCC1, PML-RARA, PRCC-TFE3, RANBP2-ALK, RBM14-PACS1, RELCH-RET, RNF130-BRAF, SDC4-ROS1, SEC16A-NOTCH1, SFPQ-TFE3, SLC26A6-PRKAR2A, SLC34A2-ROS1, SLC3A2-NRG1, SLC45A3-BRAF, SLC45A3-ELK4, SLC45A3-ETV1, SLC45A3-ETV5, SND1-BRAF, SQSTM1-ALK, SRGAP3-RAF1, SS18-SSX1, SS18-SSX2, SS18-SSX4B, SS18L1-SSX1, STRN-ALK, TADA2A-MAST1, TBL1XR1-TP63, TCEA1-PLAG1, TCF3-PBX1, TFG-ALK, TPM3-ROS1, TPR-ALK, TRIM24-BRAF, TRIM24-RET, TRIM27-RET, TRIM33-RET, VCL-ALK, WDCP-ALK, YWHAE-NUTM2A, YWHAE-NUTM2B, ZC3H7B-BCOR, ZCCHC8-ROS1 or gene fragments of any of the foregoing fusions. In embodiments, the polynucleotide fusion includes a sequence of a first region fused to a sequence of a second region at a fusion junction wherein the first region and second region include different genes. In embodiments, the polynucleotide fusion includes a gene fusion of CREBBP-SRGAP2B, DNAH14-IKZF1, ETV6-SNUPN, or ETV6-NUFIP1. The genes described herein correspond to registered genes as identified in the National Library of Medicine National Center for Biotechnology Information Catalog, accessible www.ncbi.nlm.nih.gov/gene/. Alternatively, may be a fusion gene found in known fusion gene databases, such as ChimerDB, as described in Ye Eun Jang et al., Nucleic Acids Research, Volume 48, Issue D1, 8 Jan. 2020, Pages D817-D824, or FusionGDB, as disclosed in Kim P and Zhou X. Nucleic Acids Res. 2019 Jan. 8; 47(D1):D994-D1004, each of which are incorporated herein by reference.

In embodiments, the polynucleotide fusion includes a sequence of a first region fused to a sequence of a second region at a fusion junction, wherein the first region includes an ABI1 gene or portion thereof, ACLY gene or portion thereof, ACSL3 gene or portion thereof, ACTB gene or portion thereof, ACTN4 gene or portion thereof, AFF3 gene or portion thereof, AFF4 gene or portion thereof, AGPAT5 gene or portion thereof, AGTRAP gene or portion thereof, AKAP9 gene or portion thereof, ALK gene or portion thereof, ARHGAP26 gene or portion thereof, ARHGEF12 gene or portion thereof, ARID1A gene or portion thereof, ASIC2 gene or portion thereof, ATF1 gene or portion thereof, ATIC gene or portion thereof, ATP8B2 gene or portion thereof, BBS9 gene or portion thereof, BCOR gene or portion thereof, BCR gene or portion thereof, BRAF gene or portion thereof, BTBD18 gene or portion thereof, CASP8AP2 gene or portion thereof, CBFA2T3 gene or portion thereof, CBL gene or portion thereof, CCDC6 gene or portion thereof, CD74 gene or portion thereof, CDX1 gene or portion thereof, CENPK gene or portion thereof, CEP170B gene or portion thereof, CEP89 gene or portion thereof, CIP2A gene or portion thereof, CLCN6 gene or portion thereof, COL1A1 gene or portion thereof, COL1A2 gene or portion thereof, CREB3L1 gene or portion thereof, CREB3L2 gene or portion thereof, CREBBP gene or portion thereof, CRTC3 gene or portion thereof, CXorf67 gene or portion thereof, CYP39A1 gene or portion thereof, DCTN1 gene or portion thereof, DDX5 gene or portion thereof, DHH gene or portion thereof, DNAJB1 gene or portion thereof, EEFSEC gene or portion thereof, EIF3E gene or portion thereof, EIF3K gene or portion thereof, ELK4 gene or portion thereof, ELL gene or portion thereof, EML4 gene or portion thereof, EP300 gene or portion thereof, EPC1 gene or portion thereof, EPS15 gene or portion thereof, ERBB4 gene or portion thereof, ETV1 gene or portion thereof, ETV4 gene or portion thereof, ETV5 gene or portion thereof, ETV6 gene or portion thereof, EZR gene or portion thereof, FAM131B gene or portion thereof, FBXL18 gene or portion thereof, FCHSD1 gene or portion thereof, FEV gene or portion thereof, FOXO1 gene or portion thereof, FOXO4 gene or portion thereof, FRYL gene or portion thereof, FUS gene or portion thereof, GAB2 gene or portion thereof, GABBR2 gene or portion thereof, GAS7 gene or portion thereof, GA™ gene or portion thereof, GLI1 gene or portion thereof, GLIS2 gene or portion thereof, GMDS gene or portion thereof, GMPS gene or portion thereof, GNAI1 gene or portion thereof, GOLGA5 gene or portion thereof, GOPC gene or portion thereof, GPHN gene or portion thereof, HACL1 gene or portion thereof, HAS2 gene or portion thereof, HIP1 gene or portion thereof, HOOK3 gene or portion thereof, IL6R gene or portion thereof, INTS4 gene or portion thereof, IRF2BP2 gene or portion thereof, ITPR2 gene or portion thereof, JAK2 gene or portion thereof, JAZF1 gene or portion thereof, JPT1 gene or portion thereof, KDM5A gene or portion thereof, KIF5B gene or portion thereof, KLK2 gene or portion thereof, KMT2A gene or portion thereof, KNL1 gene or portion thereof, KTN1 gene or portion thereof, LASP1 gene or portion thereof, LGR5 gene or portion thereof, LIFR gene or portion thereof, LPP gene or portion thereof, LRIG3 gene or portion thereof, LSM14A gene or portion thereof, MAML2 gene or portion thereof, MAPRE1 gene or portion thereof, MAST1 gene or portion thereof, MAST2 gene or portion thereof, MBOAT2 gene or portion thereof, MBTD1 gene or portion thereof, MCPH1 gene or portion thereof, MEAF6 gene or portion thereof, MKRN1 gene or portion thereof, MLLT1 gene or portion thereof, MLLT11 gene or portion thereof, MLLT3 gene or portion thereof, MLLT6 gene or portion thereof, MN1 gene or portion thereof, MSN gene or portion thereof, MYO1F gene or portion thereof, MYO5A gene or portion thereof, NAB2 gene or portion thereof, NCKIPSD gene or portion thereof, NCOA1 gene or portion thereof, NCOA2 gene or portion thereof, NCOA4 gene or portion thereof, NF1 gene or portion thereof, NONO gene or portion thereof, NOTCH1 gene or portion thereof, NRG1 gene or portion thereof, NRIP3 gene or portion thereof, NTN1 gene or portion thereof, NUP107 gene or portion thereof, NUP98 gene or portion thereof, NUTM2A gene or portion thereof, NUTM2B gene or portion thereof, PACS1 gene or portion thereof, PAX3 gene or portion thereof, PAX5 gene or portion thereof, PAX7 gene or portion thereof, PBX1 gene or portion thereof, PCM1 gene or portion thereof, PDE8B gene or portion thereof, PDGFB gene or portion thereof, PDGFRB gene or portion thereof, PDSSA gene or portion thereof, PHF1 gene or portion thereof, PICALM gene or portion thereof, PKD1L1 gene or portion thereof, PLA2R1 gene or portion thereof, PLAG1 gene or portion thereof, PLXND1 gene or portion thereof, PML gene or portion thereof, PRCC gene or portion thereof, PRKACA gene or portion thereof, PRKAR2A gene or portion thereof, PRKCE gene or portion thereof, RAF1 gene or portion thereof, RANBP2 gene or portion thereof, RARA gene or portion thereof, RBM14 gene or portion thereof, RBMS1 gene or portion thereof, RELCH gene or portion thereof, RET gene or portion thereof, RHEBL1 gene or portion thereof, RNF130 gene or portion thereof, RNF216 gene or portion thereof, ROS1 gene or portion thereof, RSPO2 gene or portion thereof, RUNX1 gene or portion thereof, SARNP gene or portion thereof, SDC4 gene or portion thereof, SEC16A gene or portion thereof, SFPQ gene or portion thereof, SH3GL1 gene or portion thereof, SLC26A6 gene or portion thereof, SLC34A2 gene or portion thereof, SLC3A2 gene or portion thereof, SLC45A3 gene or portion thereof, SND1 gene or portion thereof, SQSTM1 gene or portion thereof, SRGAP3 gene or portion thereof, SS18 gene or portion thereof, SS18L1 gene or portion thereof, SSX1 gene or portion thereof, SSX2 gene or portion thereof, SSX4B gene or portion thereof, STATE gene or portion thereof, STRN gene or portion thereof, SUZ12 gene or portion thereof, TADA2A gene or portion thereof, TBL1XR1 gene or portion thereof, TCEA1 gene or portion thereof, TCF3 gene or portion thereof, TET1 gene or portion thereof, TFE3 gene or portion thereof, TFG gene or portion thereof, TMCC1 gene or portion thereof, TP63 gene or portion thereof, TPM3 gene or portion thereof, TPR gene or portion thereof, TRIM24 gene or portion thereof, TRIM27 gene or portion thereof, TRIM33 gene or portion thereof, USH1G gene or portion thereof, VCL gene or portion thereof, WDCP gene or portion thereof, YWHAE gene or portion thereof, ZC3H7B gene or portion thereof, ZCCHC8 gene or portion thereof, or ZFYVE19 gene or portion thereof.

In embodiments, the polynucleotide fusion includes a sequence of a first region fused to a sequence of a second region at a fusion junction, wherein the second region includes an ABI1 gene or portion thereof, ACLY gene or portion thereof, ACSL3 gene or portion thereof, ACTB gene or portion thereof, ACTN4 gene or portion thereof, AFF3 gene or portion thereof, AFF4 gene or portion thereof, AGPAT5 gene or portion thereof, AGTRAP gene or portion thereof, AKAP9 gene or portion thereof, ALK gene or portion thereof, ARHGAP26 gene or portion thereof, ARHGEF12 gene or portion thereof, ARID1A gene or portion thereof, ASIC2 gene or portion thereof, ATF1 gene or portion thereof, ATIC gene or portion thereof, ATP8B2 gene or portion thereof, BBS9 gene or portion thereof, BCOR gene or portion thereof, BCR gene or portion thereof, BRAF gene or portion thereof, BTBD18 gene or portion thereof, CASP8AP2 gene or portion thereof, CBFA2T3 gene or portion thereof, CBL gene or portion thereof, CCDC6 gene or portion thereof, CD74 gene or portion thereof, CDX1 gene or portion thereof, CENPK gene or portion thereof, CEP170B gene or portion thereof, CEP89 gene or portion thereof, CIP2A gene or portion thereof, CLCN6 gene or portion thereof, COL1A1 gene or portion thereof, COL1A2 gene or portion thereof, CREB3L1 gene or portion thereof, CREB3L2 gene or portion thereof, CREBBP gene or portion thereof, CRTC3 gene or portion thereof, CXorf67 gene or portion thereof, CYP39A1 gene or portion thereof, DCTN1 gene or portion thereof, DDX5 gene or portion thereof, DHH gene or portion thereof, DNAJB1 gene or portion thereof, EEFSEC gene or portion thereof, EIF3E gene or portion thereof, EIF3K gene or portion thereof, ELK4 gene or portion thereof, ELL gene or portion thereof, EML4 gene or portion thereof, EP300 gene or portion thereof, EPC1 gene or portion thereof, EPS15 gene or portion thereof, ERBB4 gene or portion thereof, ETV1 gene or portion thereof, ETV4 gene or portion thereof, ETV5 gene or portion thereof, ETV6 gene or portion thereof, EZR gene or portion thereof, FAM131B gene or portion thereof, FBXL18 gene or portion thereof, FCHSD1 gene or portion thereof, FEV gene or portion thereof, FOXO1 gene or portion thereof, FOXO4 gene or portion thereof, FRYL gene or portion thereof, FUS gene or portion thereof, GAB2 gene or portion thereof, GABBR2 gene or portion thereof, GAS7 gene or portion thereof, GA™ gene or portion thereof, GLI1 gene or portion thereof, GLIS2 gene or portion thereof, GMDS gene or portion thereof, GMPS gene or portion thereof, GNAI1 gene or portion thereof, GOLGA5 gene or portion thereof, GOPC gene or portion thereof, GPHN gene or portion thereof, HACL1 gene or portion thereof, HAS2 gene or portion thereof, HIP1 gene or portion thereof, HOOK3 gene or portion thereof, IL6R gene or portion thereof, INTS4 gene or portion thereof, IRF2BP2 gene or portion thereof, ITPR2 gene or portion thereof, JAK2 gene or portion thereof, JAZF1 gene or portion thereof, JPT1 gene or portion thereof, KDM5A gene or portion thereof, KIF5B gene or portion thereof, KLK2 gene or portion thereof, KMT2A gene or portion thereof, KNL1 gene or portion thereof, KTN1 gene or portion thereof, LASP1 gene or portion thereof, LGR5 gene or portion thereof, LIFR gene or portion thereof, LPP gene or portion thereof, LRIG3 gene or portion thereof, LSM14A gene or portion thereof, MAML2 gene or portion thereof, MAPRE1 gene or portion thereof, MAST1 gene or portion thereof, MAST2 gene or portion thereof, MBOAT2 gene or portion thereof, MBTD1 gene or portion thereof, MCPH1 gene or portion thereof, MEAF6 gene or portion thereof, MKRN1 gene or portion thereof, MLLT1 gene or portion thereof, MLLT11 gene or portion thereof, MLLT3 gene or portion thereof, MLLT6 gene or portion thereof, MN1 gene or portion thereof, MSN gene or portion thereof, MYO1F gene or portion thereof, MYOSA gene or portion thereof, NAB2 gene or portion thereof, NCKIPSD gene or portion thereof, NCOA1 gene or portion thereof, NCOA2 gene or portion thereof, NCOA4 gene or portion thereof, NF1 gene or portion thereof, NONO gene or portion thereof, NOTCH1 gene or portion thereof, NRG1 gene or portion thereof, NRIP3 gene or portion thereof, NTN1 gene or portion thereof, NUP107 gene or portion thereof, NUP98 gene or portion thereof, NUTM2A gene or portion thereof, NUTM2B gene or portion thereof, PACS1 gene or portion thereof, PAX3 gene or portion thereof, PAX5 gene or portion thereof, PAX7 gene or portion thereof, PBX1 gene or portion thereof, PCM1 gene or portion thereof, PDE8B gene or portion thereof, PDGFB gene or portion thereof, PDGFRB gene or portion thereof, PDSSA gene or portion thereof, PHF1 gene or portion thereof, PICALM gene or portion thereof, PKD1L1 gene or portion thereof, PLA2R1 gene or portion thereof, PLAG1 gene or portion thereof, PLXND1 gene or portion thereof, PML gene or portion thereof, PRCC gene or portion thereof, PRKACA gene or portion thereof, PRKAR2A gene or portion thereof, PRKCE gene or portion thereof, RAF1 gene or portion thereof, RANBP2 gene or portion thereof, RARA gene or portion thereof, RBM14 gene or portion thereof, RBMS1 gene or portion thereof, RELCH gene or portion thereof, RET gene or portion thereof, RHEBL1 gene or portion thereof, RNF130 gene or portion thereof, RNF216 gene or portion thereof, ROS1 gene or portion thereof, RSPO2 gene or portion thereof, RUNX1 gene or portion thereof, SARNP gene or portion thereof, SDC4 gene or portion thereof, SEC16A gene or portion thereof, SFPQ gene or portion thereof, SH3GL1 gene or portion thereof, SLC26A6 gene or portion thereof, SLC34A2 gene or portion thereof, SLC3A2 gene or portion thereof, SLC45A3 gene or portion thereof, SND1 gene or portion thereof, SQSTM1 gene or portion thereof, SRGAP3 gene or portion thereof, SS18 gene or portion thereof, SS18L1 gene or portion thereof, SSX1 gene or portion thereof, SSX2 gene or portion thereof, SSX4B gene or portion thereof, STATE gene or portion thereof, STRN gene or portion thereof, SUZ12 gene or portion thereof, TADA2A gene or portion thereof, TBL1XR1 gene or portion thereof, TCEA1 gene or portion thereof, TCF3 gene or portion thereof, TET1 gene or portion thereof, TFE3 gene or portion thereof, TFG gene or portion thereof, TMCC1 gene or portion thereof, TP63 gene or portion thereof, TPM3 gene or portion thereof, TPR gene or portion thereof, TRIM24 gene or portion thereof, TRIM27 gene or portion thereof, TRIM33 gene or portion thereof, USH1G gene or portion thereof, VCL gene or portion thereof, WDCP gene or portion thereof, YWHAE gene or portion thereof, ZC3H7B gene or portion thereof, ZCCHC8 gene or portion thereof, or ZFYVE19 gene or portion thereof.

In embodiments, the sequence of the first region includes a sequence of a first gene (e.g., the entire gene sequence or a portion thereof), and the sequence of the second region includes a sequence of a second gene (e.g., the entire gene sequence or a portion thereof). In embodiments, the location where the first gene is connected to the second gene via an internucleosidic linkage is the fusion junction.

In embodiments, the linear nucleic acid molecules are obtained from peripheral blood samples using conventional techniques. For example, white blood cells may be separated from blood samples using convention techniques, e.g., RosetteSep kit. Blood samples may range in volume from 100 μL to 10 mL. In embodiments, blood sample volumes are in the range of from 100 μL to 2 mL. and nucleic acid molecules (e.g., DNA and/or RNA) may then be extracted from such blood sample using conventional techniques, e.g., DNeasy Blood & Tissue Kit. Optionally, subsets of white blood cells, e.g. lymphocytes, may be further isolated using conventional techniques, e.g. fluorescently activated cell sorting (FACS) or magnetically activated cell sorting (MACS). Cell-free DNA nucleic acid molecules may also be extracted from peripheral blood samples using conventional techniques as described in U.S. Pat. No. 6,258,540 or Huang et al, Methods Mol. Biol., 444: 203-208 (2008), each of which are incorporated herein by reference. For example, peripheral blood may be collected in EDTA tubes, after which it may be fractionated into plasma, white blood cell, and red blood cell components by centrifugation. DNA from the cell free plasma fraction (e.g. from 0.5 to 2.0 mL) may be extracted using a QIAamp DNA Blood Mini Kit, in accordance with the manufacturer's protocol. Various methods and commercially available kits for isolating different subpopulations of T and B cells are known in the art and include, but are not limited to, subset selection immunomagnetic bead separation or flow immunocytometric cell sorting using antibodies specific for one or more of any of a variety of known T and B cell surface markers. Illustrative markers include, but are not limited to, one or a combination of CD2, CD3, CD4, CD8, CD14, CD19, CD20, CD25, CD28, CD45RO, CD45RA, CD54, CD62, CD62L, CDw137 (41BB), CD154, GITR, FoxP3, CD54, and CD28. For example, and as is known to the skilled person, cell surface markers, such as CD2, CD3, CD4, CD8, CD14, CD19, CD20, CD45RA, and CD45RO may be used to determine T, B, and monocyte lineages and subpopulations in flow cytometry. Similarly, forward light-scatter, side-scatter, and/or cell surface markers such as CD25, CD62L, CD54, CD137, CD154 may be used to determine activation state and functional properties of cells. Linear nucleic acid molecules (e.g., DNA or RNA) may be extracted from cells in a sample, such as a sample of blood or lymph or other sample from a subject known to have or suspected of having a disease (e.g., a lymphoid hematological malignancy), using standard methods or commercially available kits known in the art.

In embodiments, the fusion junction can be an unknown fusion junction event, since no prior knowledge of the exact nature of the genomic rearrangement is needed for the methods disclosed herein to be able to detect and characterize the fusion. In embodiments, only the sequence of a first region is known before circularization. In embodiments, only the sequence of a second region is known before circularization.

In embodiments, the first and second regions are located on the same chromosome. In embodiments, the first and second regions are located on different chromosomes.

In another aspect is provided a method for detecting a fusion gene in a sample from a subject, the method including: contacting a plurality of circular template polynucleotides with a plurality of primers, and binding a primer to each circular template polynucleotide thereby forming primed circular templates, wherein one or more circular template polynucleotides include a fusion gene and one or more circular template polynucleotides do not include the fusion gene; contacting the primed circular templates with a plurality of blocking elements and binding a blocking element to the primed templates that do not include said fusion gene, wherein the blocking element is an oligonucleotide; extending a primer bound to a circular template polynucleotide including a fusion gene with a polymerase to generate an extension product including a complement of said fusion gene; and sequencing the extension product, or a complement thereof, thereby detecting the fusion gene. In embodiments, the plurality of circular template polynucleotides include a first circular template polynucleotide including a sequence from an IGH locus, a second circular template polynucleotide including a sequence from an IGK locus, and a third circular template polynucleotide including a sequence from an IGL locus.

### III. Compositions and Kits

In an aspect is provided a composition including a first primer and a second primer. In embodiments, the composition further includes a blocking element. In embodiments, the composition further includes an annealing solution (alternatively referred to herein as a hybridization buffer or hybridization solution). In embodiments, the annealing solution includes an aqueous solution which may contain buffers (e.g., saline-sodium citrate (SSC), tris(hydroxymethyl) aminomethane or “Tris”), aqueous salts (e.g., KCl or (NH₄)₂SO₄)), chelating agents (e.g., EDTA), detergents, surfactants, crowding agents, or stabilizers (e.g., PEG, Tween-20, BSA). In embodiments, the annealing solution includes Tris and is maintained at a pH from about 8.0 to about 9.0. In embodiments, the composition includes an extension solution. In embodiments, the extension solution includes an aqueous solution which may contain buffers (e.g., saline-sodium citrate (SSC), tris(hydroxymethyl)aminomethane or “Tris”), aqueous salts (e.g., KCl or (Mg)₂SO₄)), nucleotides, polymerases, detergents, chelators (e.g., EDTA), surfactants, crowding agents, or stabilizers (e.g., PEG, Tween-20, BSA). In embodiments, the composition further includes an additive that lowers a DNA denaturation temperature. In embodiments, the composition includes an additive such as betaine, dimethyl sulfoxide (DMSO), ethylene glycol, formamide, glycerol, guanidine thiocyanate, 4-methylmorpholine 4-oxide (NMO), or a mixture thereof. In embodiments, the composition further includes a denaturant. The denaturant may be acetic acid, hydrochloric acid, nitric acid, formamide, guanidine, sodium salicylate, sodium hydroxide, dimethyl sulfoxide (DMSO), propylene glycol, urea, or a mixture thereof.

In embodiments, the composition includes a circularizing solution (e.g., a circularizing agent). In embodiments, the circularizing solution includes a circularizing ligase, such as CircLigase™, Taq DNA Ligase, HiFi Taq DNA Ligase, T4 ligase, or Ampligase® DNA Ligase. In embodiments, the circularizing solution includes a splint primer. A “splint primer” is used according to its plain and ordinary meaning and refers to a primer having 2 or more sequences complementary to two or more portions of a template polynucleotide. In embodiments, the two sequences are adapter sequences wherein one adapter sequences binds (i.e., hybridizes) to a 5′ portion of the template polynucleotide and the other adapter binds (i.e., hybridizes) to a 3′ portion of the template polynucleotide. In embodiments, the circularizing solution includes a crowding agent, such as PEG (e.g., 20-25% PEG-8000). In embodiments, the circularizing solution includes polyethylene glycol (PEG), such as PEG 4000 or PEG 6000, Dextran, and/or Ficoll.

In an aspect is provided a kit including: a circularizing agent, wherein the circularizing agent is capable of joining the 5′ and 3′ ends of a linear nucleic acid molecule; optionally, a blocking element capable of binding to one or more circular polynucleotides; a first primer and a second primer; and a polymerase. In embodiments, the first primer and the second primer form a primer set. In embodiments, the kit includes a plurality of primer sets. In embodiments, the kit includes 5, 10, 20, 25, 50 or more primer sets.

In embodiments, the kit includes at least 22 different primers, for example a forward primer (1 F), and six reverse primers (6 R) for the IGH locus; three forward (3 F), and six reverse (6 R) for the IGK locus; and one forward (1 F), and five reverse primers (5 R) for the IGL locus. In embodiments, the kit includes about 18 elements (i.e., 18 blocking elements targeting 18 different regions). In embodiments, the kit includes primers targeting 7 different sequences for the IGH locus. In embodiments, the kit includes primers targeting 9 different sequences for the IGK locus. In embodiments, the kit includes primers targeting 6 different sequences for the IGL locus. In embodiments, the kit includes a plurality of different populations of blocking elements, each population of blocking elements binding to a specific sequence.

In embodiments, the splint primer is about 5 to about 25 nucleotides in length. In embodiments, the splint primer is about 10 to about 40 nucleotides in length. In embodiments, the splint primer is about 5 to about 100 nucleotides in length. In embodiments, the splint primer is about 20 to 200 nucleotides in length. In embodiments, the splint primer is about or at least about 5, 6, 7, 8, 9, 10, 12, 15, 18, 20, 25, 30, 35, 40, 50 or more nucleotides in length. In embodiments, the splint primer is about or at least about 10 nucleotides in length. In embodiments, the splint primer is about or at least about 15 nucleotides in length. In embodiments, the splint primer is about or at least about 25 nucleotides in length.

In an aspect is provided a kit containing the component necessary to perform the methods as described herein, including embodiments. Generally, the kit includes one or more containers providing a composition, and one or more additional reagents (e.g., a buffer suitable for polynucleotide extension). The kit may also include a template nucleic acid (DNA and/or RNA), one or more primer polynucleotides, nucleotides (including, e.g., deoxyribonucleotides, ribonucleotides, labeled nucleotides, and/or modified nucleotides), buffers, salts, and/or labels (e.g., fluorophores). In embodiments, the kit further includes instructions. In embodiments the kit includes one or more enclosures (e.g., boxes, bottles, or cartridges) containing the relevant reaction reagents and/or supporting materials. In embodiments, the kit includes components useful for circularizing template polynucleotides using chemical ligation techniques. In embodiments, the kit includes components useful for circularizing template polynucleotides using a ligation enzyme (e.g., CircLigase™ enzyme, Taq DNA Ligase, HiFi Taq DNA Ligase, T4 ligase, or Ampligase DNA Ligase). In embodiments the ligation enzyme is an RNA-dependent DNA ligase (e.g., SplintR ligase). For example, such a kit further includes the following components: (a) reaction buffer for controlling pH and providing an optimized salt composition for a ligation enzyme (e.g., CircLigase™ enzyme, Taq DNA Ligase, HiFi Taq DNA Ligase, T4 ligase, or Ampligase DNA Ligase), and (b) ligation enzyme cofactors. In embodiments, the kit further includes instructions for use thereof.

In embodiments, the kit includes a plurality of primers, wherein the primers are capable of hybridizing to the linear nucleic acid molecules. Nucleic acid hybridization techniques may be used to assess hybridization specificity of the primers described herein. Hybridization techniques are well known in the art, for example, suitable moderately stringent conditions for testing the hybridization of a polynucleotide as provided herein with other polynucleotides include prewashing in a solution of 5×SSC, 0.5% SDS, 1.0 mM EDTA (pH 8.0); hybridizing at 50° C.-60° C., 5×SSC; followed by washing twice at 65° C. for 20 minutes with each of 2×, 0.5× and 0.2×SSC containing 0.1% SDS.

In embodiments, the kit includes a primer set. In embodiments, the kit includes a plurality of primer sets. The number of primers in a first set may be the same or different than the number of primers in a second set. A “primer set” or “primer pair”, as used herein, refers to two or more primers targeting two or more regions of a polynucleotide. Typically, a primer set includes a first primer that hybridizes to a 5′ portion of the polynucleotide and a second primer that hybridizes to a 3′ portion of a polynucleotide. For example, a forward and reverse primer flank the target region of a polynucleotide, and collectively the forward primer and reverse primer are considered a primer set. In embodiments, the kit includes a first set of “upstream” or “forward” primers, and a second set of “downstream” or “reverse” primers. In embodiments, kits further include forward and reverse primer sets specific for amplifying recombined nucleic acids encoding IgH(VDJ), IgH(DJ) and IgK. In some embodiments, kits further include forward and reverse primer sets specific for amplifying recombined nucleic acids encoding TCRβ, TCRδ and TCRγ. In embodiments, the kit includes a plurality of V segment primers (i.e., primers having complementary sequences to the V encoding region) and a plurality of J segment primers (i.e., primers having complementary sequences to the J encoding region), wherein the plurality of V segment primers and the plurality of J segment primers amplify substantially all combinations of the V and J segments of a rearranged immune receptor locus. By substantially all combinations is meant at least 95%, 96%, 97%, 98%, 99% or more of all the combinations of the V and J segments of a rearranged immune receptor locus. In certain embodiments, the plurality of V segment primers and the plurality of J segment primers amplify all of the combinations of the V and J segments of a rearranged immune receptor locus. In embodiments, primers may include or at least about 15 nucleotides long that has the same sequence as, or is complementary to, a 15 nucleotide long contiguous sequence of the target V- or J-segment (i.e., portion of genomic polynucleotide encoding a V-region or J-region polypeptide). Longer primers, e.g., those of about 16, 17, 18, 19, 20, 21, 22, 23, 24, 25, 26, 27, 28, 29, 30, 31, 32, 33, 34, 35, 36, 37, 38, 39, 40, 45, or 50, nucleotides long that have the same sequence as, or sequence complementary to, a contiguous sequence of the target V- or J-region encoding polynucleotide segment, may also be used in the methods and kits described herein. In embodiments, the kit includes inward facing primers. In embodiments, the kit includes outward facing primers. A primer set may include more than two distinct primers, for example a forward primer (1 F), and six reverse primers (6 R) for the IGH locus, collectively is a primer set for the IGH locus.

In embodiments, the kit further includes forward and reverse primer sets for amplifying one or more target sequences including a single-nucleotide variant, an insertion, a deletion, an internal tandem duplication, and/or a copy number variant. In embodiments, the kit further includes forward and reverse primer sets for amplifying one or more target sequences including one or more single-nucleotide variants, one or more insertions, one or more deletions, one or more internal tandem duplications, or one or more copy number variants.

In embodiments, the kit includes at least 2, 4, 6, 8, 10, 20, 40, 60, 80, 100, 120, 140, 160, 180, 200, or more primer sets. In embodiments, the kit includes between 2 to 10, between 10 to 40, between 40 to 80, between 80 to 150, between 150 to 300, or more primer sets. The number of primer sets provided in the kit may be customized for a specific application, for example, detecting a known number of recombined nucleic acids, and/or for detecting a known number of single-nucleotide variants, insertions, deletions, internal tandem duplications, and/or copy number variants. In embodiments, the kit includes multiple (e.g., a plurality) primer sets for amplifying a single genomic feature.

In embodiments, the kit includes a sequencing polymerase, and one or more amplification polymerases. In embodiments, the sequencing polymerase is capable of incorporating modified nucleotides. In embodiments, the polymerase is a DNA polymerase. In embodiments, the DNA polymerase is a Pol I DNA polymerase, Pol II DNA polymerase, Pol III DNA polymerase, Pol IV DNA polymerase, Pol V DNA polymerase, Pol β DNA polymerase, Pol μ DNA polymerase, Pol λ DNA polymerase, Pol σ DNA polymerase, Pol α DNA polymerase, Pol δ DNA polymerase, Pol ε DNA polymerase, Pol η DNA polymerase, Pol ι DNA polymerase, Pol κ DNA polymerase, Pol ζ DNA polymerase, Pol γ DNA polymerase, Pol θ DNA polymerase, Pol ν DNA polymerase, or a thermophilic nucleic acid polymerase (e.g., Therminator γ, 9° N polymerase (exo-), Therminator II, Therminator III, or Therminator IX). In embodiments, the DNA polymerase is a thermophilic nucleic acid polymerase. In embodiments, the DNA polymerase is a modified archaeal DNA polymerase. In embodiments, the polymerase is a reverse transcriptase. In embodiments, the polymerase is a mutant P. abyssi polymerase (e.g., such as a mutant P. abyssi polymerase described in WO 2018/148723 or WO 2020/056044, each of which are incorporated herein by reference for all purposes). In embodiments, the kit includes a strand-displacing polymerase. In embodiments, the kit includes a strand-displacing polymerase, such as a phi29 polymerase, Bst polymerase (e.g., Bst Lf), phi29 mutant polymerase or a thermostable phi29 mutant polymerase.

In embodiments, the kit includes a buffered solution. Typically, the buffered solutions contemplated herein are made from a weak acid and its conjugate base or a weak base and its conjugate acid. For example, sodium acetate and acetic acid are buffer agents that can be used to form an acetate buffer. Other examples of buffer agents that can be used to make buffered solutions include, but are not limited to, Tris, bicine, tricine, HEPES, TES, MOPS, MOPSO and PIPES. Additionally, other buffer agents that can be used in enzyme reactions, hybridization reactions, and detection reactions are known in the art. In embodiments, the buffered solution can include Tris. With respect to the embodiments described herein, the pH of the buffered solution can be modulated to permit any of the described reactions. In some embodiments, the buffered solution can have a pH greater than pH 7.0, greater than pH 7.5, greater than pH 8.0, greater than pH 8.5, greater than pH 9.0, greater than pH 9.5, greater than pH 10, greater than pH 10.5, greater than pH 11.0, or greater than pH 11.5. In other embodiments, the buffered solution can have a pH ranging, for example, from about pH 6 to about pH 9, from about pH 8 to about pH 10, or from about pH 7 to about pH 9. In embodiments, the buffered solution can include one or more divalent cations. Examples of divalent cations can include, but are not limited to, Mg²⁺, Mn²⁺, Zn²⁺, and Ca²⁺. In embodiments, the buffered solution can contain one or more divalent cations at a concentration sufficient to permit hybridization of a nucleic acid. In embodiments, the kit includes an annealing solution, an extension solution, and a chemical denaturant. In embodiments, kits further include internal standards, including a plurality of nucleic acids having lengths and compositions representative of the target nucleic acids, wherein the internal standards are provided in known concentrations.

The kit may further include one or more other containers including PCR and sequencing buffers, diluents, subject sample extraction tools (e.g. syringes, swabs, etc.), and package inserts with instructions for use. In addition, a label can be provided on the container with directions for use, such as those described above; and/or the directions and/or other information can also be included on an insert which is included with the kit; and/or via a website address provided therein. The kit may also include laboratory tools such as, for example, sample tubes, plate sealers, microcentrifuge tube openers, labels, magnetic particle separator, foam inserts, ice packs, dry ice packs, insulation, etc. The kits may further include pre-packaged or application-specific functionalized substrates as described herein for use in amplification and/or detection of the library molecules. In embodiments, the substrate may include a surface suitable for performing sequencing reactions therein.

In an aspect is provided a kit, wherein the kit includes i) an enzyme to circularize nucleic acids (e.g., a circularizing agent as described herein, such as a thermostable ATP-dependent ligase that catalyzes intramolecular ligation of ssDNA templates having a 5′-phosphate and a 3′-hydroxyl group); ii) a plurality of oligonucleotide primers; iii) optionally, a plurality of blocking elements (e.g., a blocking element as described herein); iv) a polymerase (e.g., a non-strand displacing polymerase, such as Phusion®); and v) a plurality of nucleotides (e.g., dNTPs for amplification, extension, and/or sequencing in a suitable buffer).

In embodiments, the plurality of oligonucleotide primers includes at least 7 primers (for the IGH locus. In embodiments, a subset of the plurality of primers all targeting the Joining gene. In embodiments, the plurality of oligonucleotide primers includes at least two distinct populations of primers (e.g., a first and a second primer pair, or a primer set). In embodiments, the plurality of oligonucleotide primers includes about 1, 2, 3, 4, 5, 10, 15, 25, 50, 75, 100, 150, 200, 250, 300, 400, 500, 600, 700, 800, 900, or 1000 different primer sets. In embodiments, each primer set is provided in a concentration of about 25 nM to about 200 nM. In embodiments, each primer set is provided in a concentration of about 100 nM. In embodiments, there is one blocking element per set provided.

In embodiments, the plurality of blocking elements includes at least two distinct populations of blocking elements. In embodiments, the blocking elements include at least 6 different blocking elements (e.g., for the IGH locus, 6 blocking elements are used for targeting each Joining gene).

In embodiments, the polymerase is Q5 High-Fidelity DNA Polymerase, Taq DNA polymerase, Bst DNA polymerase, T7 DNA polymerase, Sulfolobus DNA Polymerase, or DNA Polymerase I.

In embodiments, the kit further includes a fragmentation enzyme (e.g., an enzyme capable of fragmenting a high molecular weight DNA sample into −200-300 bp DNA fragments). In some embodiments, the primers are used in a single pool PCR reaction. In other embodiments the primers are used in a multi-pool PCR reaction.

In embodiments, the kit further includes a restriction enzyme or CRISPR/Cas9 protein for use in depleting wild type (WT) DNA circles. For example, in embodiments, the WT DNA specific depletion would be mediated by WT DNA specific oligonucleotides (e.g. the blocking elements), that is, the Cas9 would be guided by ‘blocker’ guide RNAs (i.e., the blocking element is a guide RNA) that would linearize the WT DNA circles, preventing exponential amplification in the subsequent step. In embodiments, the kit further includes a plurality of adapters. In embodiments, the kit further includes instructions.

In embodiments, the kit further includes a blocking element including a biotin. In embodiments, the kit further includes a blocking element including a restriction site. In embodiments, the kit further includes a methylation sensitive restriction enzyme (e.g., NotI, NaeI, NsbI, SalI, HapII, or HaeII).

In an aspect is provided a microfluidic device, wherein the microfluidic device is capable of performing any of the methods described herein, including embodiments. The microfluidic device is applicable for amplifying, processing, and/or detecting samples of analytes of interest in a flow cell. Within this application the fluidic system is made in reference to nucleic acid sequencing (i.e., a genomic instrument) which allows for the sequencing of nucleic acid molecules. However, the techniques disclosed herein may be applied to any system making use of reaction vessels, such as flow cells, for detection of analytes of interest, and into which solutions are introduced during preparation, reaction, detection, or any other process on or within the reaction vessel. The term “microfluidic device” means an integrated system of one or more chambers, ports, and channels that are interconnected and in fluid communication and designed for carrying out an analytical reaction or process, either alone or in cooperation with an appliance or instrument that provides support functions, such as sample introduction, fluid and/or reagent driving means, temperature control, detection systems, data collection and/or integration systems, for the purpose of determining the nucleic acid sequence of a template polynucleotide.

In embodiments, the device includes a light source that illuminates a sample, an objective lens, and a sensor array (e.g., complementary metal-oxide-semiconductor (CMOS) array or a charge-coupled device (CCD) array). Nucleic acid sequencing devices may further include valves, pumps, and specialized functional coatings on interior walls. For example, the microfluidic device is a nucleic acid sequencing device provided by Singular Genomics™ (e.g., G4™ sequencing platform), Illumina™, Inc. (e.g. HiSeq™, MiSeq™, NextSeq™, or NovaSeq™ systems), Life Technologies™ (e.g. ABI PRISM™, or SOLiD™ systems), Pacific Biosciences (e.g. systems using SMRT™ Technology such as the Sequel™ or RS II™ systems), or Qiagen (e.g. Genereader™ system).

### EXAMPLES

### Example 1. Fusion Detection by Template Circularization and Multiplex PCR

Gene fusions are somatic alterations associated with up to 20% of cancer morbidity and having oncogenic roles in hematological, soft tissue, and solid tumors (Foltz S M et al. Nature Comm. 2020; 11:2666). Translocations, copy number changes, and inversions can lead to fusions, dysregulated gene expression, and novel molecular functions. Next generation sequencing (NGS) approaches to gene fusion detection may employ untargeted sequencing (e.g., whole genome or whole transcriptome sequencing) or targeted sequencing of fusion genes of interest. Targeted approaches for gene fusion detection enable simplified analysis and reduced cost and have accordingly become a leading approach for clinical applications. Popular methods for targeted sequencing of gene fusions include multiplex PCR, where primer sets are designed to generate PCR amplicons spanning known breakpoint junctions (e.g., Maher C A et al. Nature. 2009; 458(7234): 97-101 and Oncomine tests); anchored multiplex PCR (AMP), where one or more targeting primers are used in conjunction with a ligated universal primer adapter to enable PCR amplification of breakpoints of interest (e.g., kits offered by ArcherDx, Inc.); and methods utilizing hybridization capture to enrich for breakpoint regions of interest. Of the targeted approaches, multiplex PCR provides high sensitivity and sequencing efficiency but cannot identify fusions involving novel breakpoints and partners; AMP enables detection of known and novel fusions, but has a relatively higher input requirement and more complex workflow that is generally restricted to the analysis of RNA; hybrid capture has a relatively complex workflow and reduced sensitivity compared to PCR based approaches. For both targeted and untargeted approaches, robustness to sample degradation is often of paramount importance owing to the widespread use of FFPE preserved tissue and cfDNA as input material. Thus, there exists a need for methods to enable high sensitivity targeted analysis of gene fusions, with minimal workflow complexity and input requirement, and a robustness to highly degraded materials.

Gene fusions represent promising targets for cancer therapy. Many novel compounds inhibiting rearranged, fusion proteins have been developed. Thus, a reliable detection of these fusions is essential in patient treatment. Previous attempts to identify fusion genes have been described. For example, U.S. Patent Application No. US 2021/0147834 (referred to as the 834 method) presents methods to identify fusion genes from a sample containing the corresponding wild type gene, wherein this method required the generation of a circle cDNA template; hybridization of a circle cleaving primer containing an endonuclease recognition site to the circle cDNA template containing the wild type gene, which facilitates the cleavage of the circle cDNA containing the wild type gene in presence of an endonuclease; and the amplification of the circle cDNA containing the fusion gene. However, there exist some key limitations of the method described in the 834 method. Firstly, the utility of the method of the 834 method is limited to RNA and cDNA. In contrast, the method as described herein is suitable for genomic DNA (gDNA), RNA, and cDNA. Secondly, the method of the 834 method requires a circle cleaving primer containing an endonuclease recognition site to preferentially deplete the non-fusion circle cDNA; this requirement restricts the utility of the method of the 834 method as some targets will not have suitable endonuclease recognition sites to complementarily hybridize with the endonuclease recognition site in the circle cleaving primer sequence. Furthermore, use of an endonuclease renders the method of the 834 method unsuitable for the evaluation of a large amplicon panel as the endonuclease recognition site is likely to be found within other amplicons. Equally important, depending on the type of endonuclease, the buffer used with the endonuclease might be incompatible with downstream PCR steps, and therefore, would require an additional clean-up step and at the risk of sample loss. Finally, high variability in the breakpoint regions could limit the design of a circle cleaving primer. The 834 method illustrated the use of the method described therein to the enrichment of a Bcr (exon 6)-ABL1 (exon 2) fusion in the presence of a circle cleaving primer that targeted exon 1 of ABL1 gene and contained a Hind III recognition sequence. However, it has been reported that the breakpoint of ABL1 could occur upstream of exon 1b, downstream of exon 1a, or between exons 1b and 1a of ABL1 (Quintas-Cardama et al. Blood. 2009 Feb. 19; 113(8): 1619-1630 and therefore, the use of a circle cleaving primer targeting exon 1 of ABL1 as described in the 834 method could reduce the accuracy of the detection of Bcr-ABL1 fusion gene. The method as described herein is distinct from the 834 method as it can be applied to a broader scope of target nucleic acids and enriches the fusion circle cDNA templates while avoiding the drawbacks associated with the use of the circle cleaving primer with an endonuclease recognition site.

The compositions and methods described herein provide sequencing-efficient solutions to achieve targeted sequencing of genetic variations such as SNVs, insertion/deletions, and gene fusions, including those involving novel partners and deriving from novel breakpoints. The methods enable a high sensitivity of detection from degraded materials with a simplified workflow. Importantly, the methods may be applied to analyze nucleic acids extracted in bulk from a sample source (e.g., cfDNA from plasma, nucleic acids from an FFPE preserved tissue specimen, or nucleic acids extracted from peripheral blood leukocytes) or material derived from common single cell library preparation systems. Detailed herein in various embodiments, the method includes the steps of (1) circularizing nucleic acids derived from a sample; (2) amplifying circularized nucleic acids deriving from one or more targets of interest; and (3) analyzing the amplified fragments via next generation sequencing (NGS).

In one embodiment of the method described herein, a workflow is presented to achieve targeted amplification of nucleic acids for the analysis of gene fusions, including those involving novel partners or breakpoints. Briefly, the workflow begins with extracting bulk nucleic acids from the sample. RNA, DNA, or total nucleic acids (RNA and DNA) may be extracted using methods known in the art. If RNA is extracted, the RNA may be converted to cDNA using methods known in the art (e.g., oligo-dT cDNA synthesis, cDNA synthesis via random hexamers, targeted cDNA synthesis via gene specific primers). If utilizing cell free DNA (cfDNA), no fragmentation may be required. DNA molecules may be optionally fragmented to an average length of approximately 150 base pairs. Fragmentation may be accomplished via methods known in the art (e.g., enzymatic fragmentation, acoustic fragmentation). Next, ssDNA fragments are circularized via enzymatic ligation of the 5′ and 3′ ends using methods known in the art (e.g., CircLigase™) or a method described herein. In some embodiments, circularization is facilitated by denaturing double-stranded nucleic acids prior to circularization. In embodiments, prior to circularization, the linear DNA fragments are A-tailed (e.g., A-tailed using Taq DNA polymerase). Residual linear DNA molecules may be optionally digested. This may be accomplished via methods known in the art (e.g., treating with an Exo I and/or Exo III).

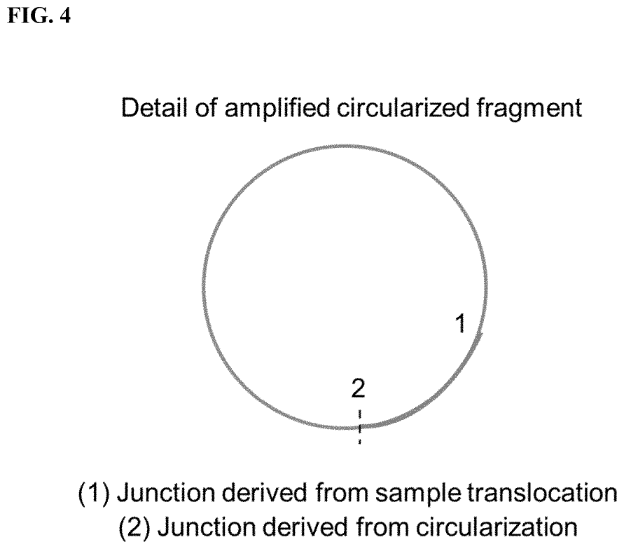

Following circularization, nucleic acids are amplified from a gene fusion of interest using outward facing oligonucleotide primers targeting a fusion gene partner of interest adjacent to the expected breakpoint location (i.e., targeting one of the two suspected genes). A 5′ blocking element (e.g., a non-extendable oligonucleotide) that specifically binds to the sequence of the unrearranged fusion gene partner of interest adjacent and opposite to the expected breakpoint junction may optionally be included (FIGS. 1-3). The blocking element does not bind templates containing a translocation at the expected breakpoint. In general, the blocking element has a melting temperature (Tm) similar to or higher than the outward facing oligonucleotide primers, to ensure that it can bind and prevent extension of the primers. The distance of the optional 5′ blocking may be within about 50 bp of the fusion junction. PCR results in amplification of both fusion templates and unrearranged templates. Resultant amplicons may contain both a junction derived from template circularization (“circularization junction”) and a junction corresponding to the sample breakpoint (FIG. 4). The circularization junction may be used to quantify the number of template copies and optionally perform error correction.

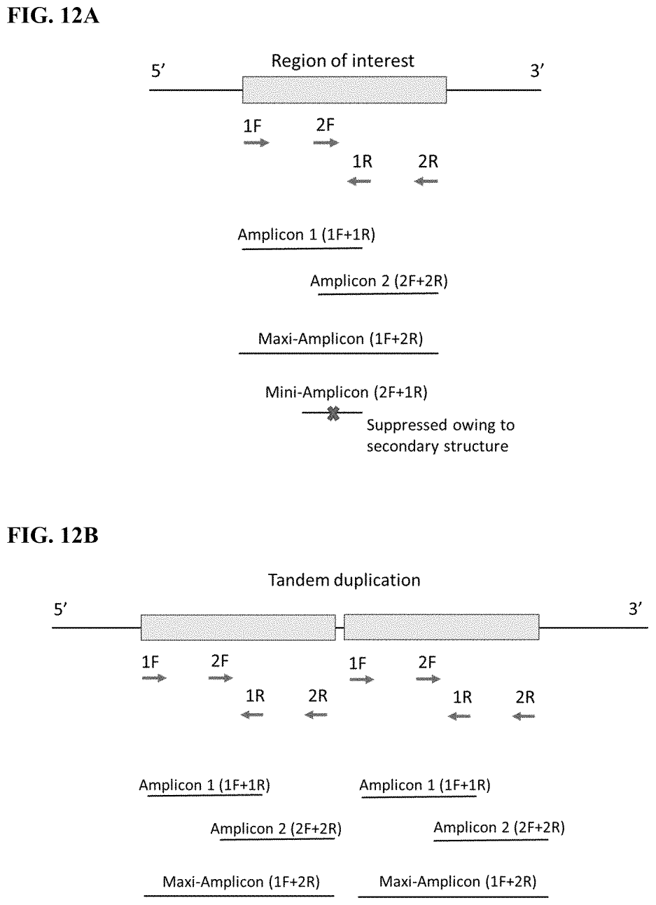

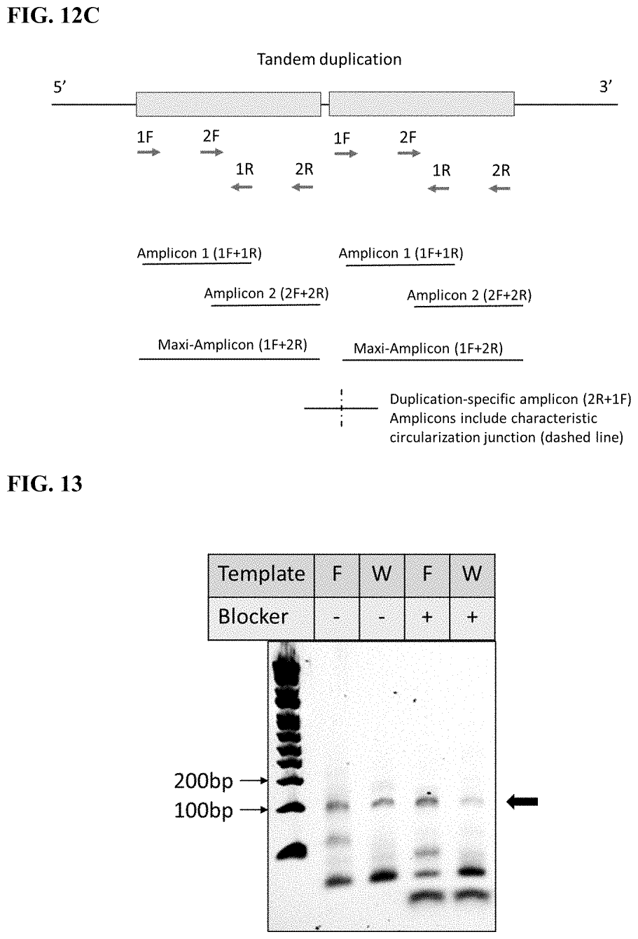

Amplification of unfused genes: As an internal control and to further assess the relative abundance of fusion gene nucleic acids amplified, amplification of nucleic acids derived from one or more unrearranged (e.g., control) templates of interest may be performed within the same PCR reaction using outward facing primers. Selection of the primer sequence is important, since it is known that the templates of the fusion genes differ from the corresponding wild type templates at the 3′ and 5′ ends (Latysheva et al. Nucleic Acids Res. 2016 Jun. 2; 44(10): 4487-4503). Alternatively, in some embodiments it is advantageous to include a positive control to avoid false negative results. Further, in some embodiments, outward facing primers are included to target regions of the human genome or cDNA where clinically relevant SNVs, insertion/deletions or copy number variants are known to occur. In some embodiments, regions of interest may include cDNA derived from genes having misregulated expression in cancer, and/or genes whose expression is largely invariant (e.g., housekeeping genes) to aid in analysis of gene expression. Analysis of such targets may be performed within the same PCR reaction using outward facing primers. In yet other embodiments, outward facing primers targeting fusions of interest are used in conjunction with inward facing primers targeting regions of interest of the human genome or cDNA where clinically relevant SNVs, insertion/deletions, internal tandem duplications or copy number variants are known to occur, as part of a multiplex PCR panel. FIG. 12A illustrates an embodiment wherein two pairs of overlapping inward facing primers (e.g., 1F and 1R, and 2F and 2R) are used to amplify a target region, resulting in three amplification products (e.g., three PCR products: Amplicon 1 (amplification product of the 1F and 1R primer pair), Amplicon 2 (amplification product of the 2F and 2R primer pair), and a Maxi-Amplicon (amplification product of the 1F and 2R primer pair), as described in U.S. Pat. Pub. US2016/0340746, which is incorporated herein by reference in its entirety. Production of a Mini-Amplicon by the 2F and 1R primer pair is suppressed due to stable secondary structure resulting in less efficient amplification. The products of the amplification reaction with the overlapping inward facing primers are identical whether a linear or circularized template is used.

By “overlapping primers,” it is meant that, for example, two pairs of primers (e.g., 1F and 1R, and, 2F and 2R in FIG. 12A) have an overlapping target region of the target nucleic acid (e.g., the 1F and 1R amplification product will include a sequence portion that is also included in the 2F and 2R amplification product. For example, as shown in FIG. 12A, the 2F primer is located upstream and adjacent to the 1R primer, while the 2R primer is located downstream of the 1R primer, thereby leading to overlapping amplification products, wherein the region contacted by and between the 2F and 1R primers will be shared between Amplicon 1 and Amplicon 2.

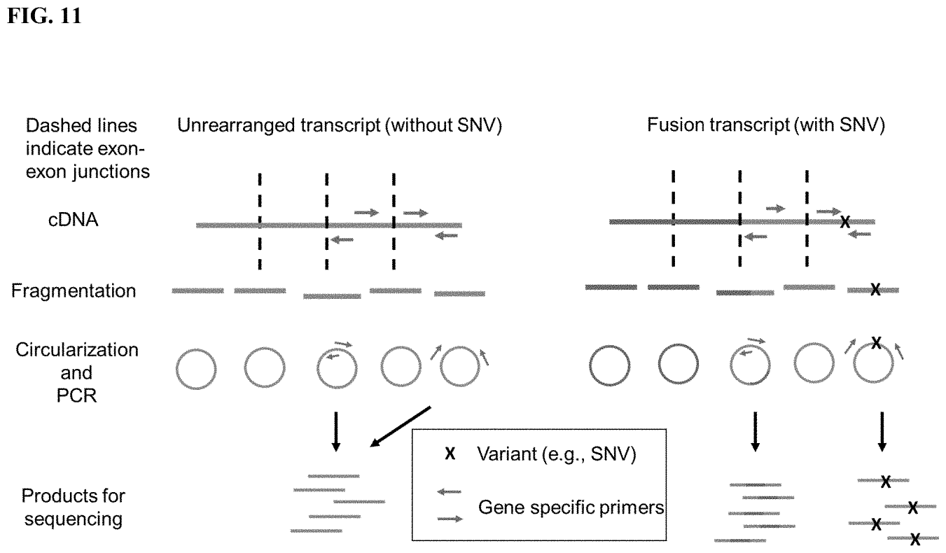

FIG. 12B illustrates the expected amplification products from an embodiment wherein amplification of an internal tandem duplication is performed with the primer pairs of FIG. 12A (e.g., 1F and 1R, and 2F and 2R) when using a linear template. The amplification products are identical to those of the non-duplicated template in FIG. 12A (e.g., Amplicon 1, Amplicon 2, and the Maxi-Amplicon), precluding detection of the tandem duplication event. FIG. 11C illustrates the expected amplification products from an embodiment wherein amplification of an internal tandem duplication is performed with the primer pairs of FIG. 12A (e.g., 1F and 1R, and 2F and 2R) when using a circularized template. The amplification products now include a duplication-specific amplicon (e.g., an amplification product of the 2R and 1F primer pair). The duplication-specific amplicon is identified both by the unique pair of primers appearing in the amplicon and the presence of a circularization junction within the amplicon (denoted by the dashed line). In such a scenario, inverse PCR products may be formed that unambiguously identify a duplication event.

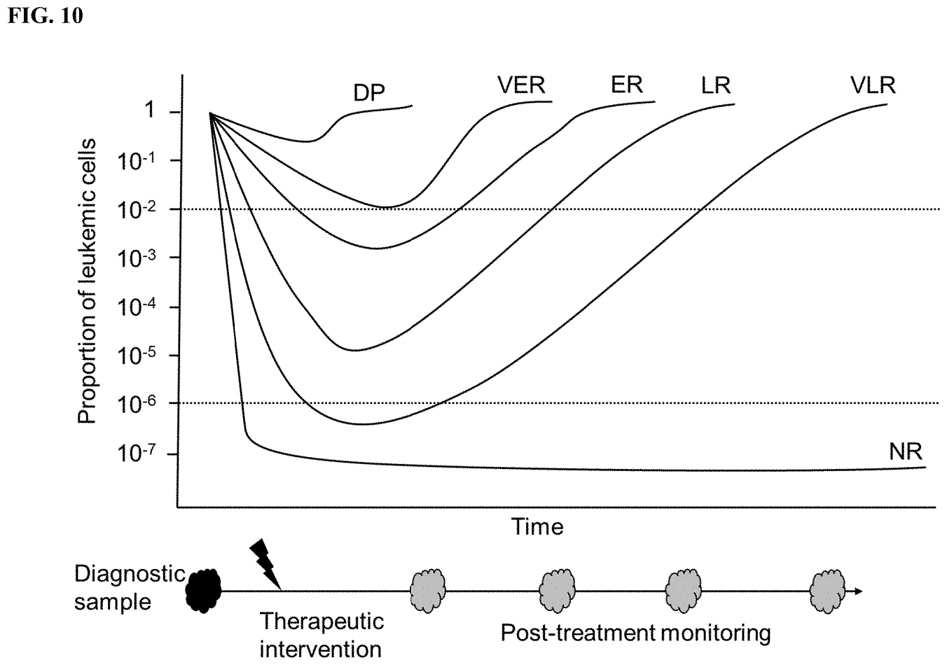

Inward facing primers: While outward facing primers are especially useful for determining novel gene fusion partners, it may also be useful to perform targeted gene sequencing to identify somatic mutations (e.g., SNPs associated with a perturbed cellular state). Specifically, inward facing primers (e.g., standard PCR primers) are used that target a region of interest that contains a known somatic alteration associated with a diseased state. In embodiments, outward facing primers targeting fusions of interest are used in conjunction with inward facing primers targeting regions of the human genome or cDNA where clinically relevant SNVs or SNPs, insertion/deletions, or copy number variants (CNVs) are known to occur, for example, as part of a multiplex PCR panel (see, e.g., FIG. 10). Inward facing primers, similar to outward facing primers, contain a target specific sequence, and optionally, a sequence for downstream library preparation and analysis. In embodiments, the inward facing primers amplify regions of interest in the absence of fusion genes (e.g., inward facing primers are used targeting a region with known somatic mutations that is distinct from an exon breakpoint and/or fusion gene partner). In embodiments, the inward facing primers target regions of interest in a fusion gene transcript (e.g., the inward facing primers target one or more regions of a fusion gene transcript, wherein the one or more regions may be in different or the same gene). In embodiments, the inward facing primers target a different gene than the outward facing primers (e.g., the inward facing primers target one gene of a fusion transcript, while the outward facing primers target the other gene of the fusion transcript). Inward and outward facing primers may, for example, be included in the same amplification reaction, or they may be pooled into individual reactions (e.g., an amplification reaction consisting only of inward facing primers and an amplification reaction consisting only of outward facing primers, wherein each amplification reaction uses the same circularized template).

Variations of the blocking element: The blocking element selectively binds to unrearranged template to inhibit extension of the primer sequences by the polymerase. In some embodiments, the blocking element consists of an oligomer (“blocking oligomer”) having an inverted 3′ dT, a 3′ dideoxycytidine, a reversibly terminated 3′ modification, or other modifications of the 3′ chain to prevent 3′ extension by a polymerase and is used in conjunction with a non-strand displacing polymerase. In some embodiments, the blocking oligomer contains one or more non-natural bases that facilitate hybridization of the blocker to the target sequence (e.g., LNA bases). In some embodiments, the blocking oligomer contains other modified bases to increase resistance to exonuclease digestion (e.g., one or more phosphorothioate bonds). The blocking element need not be an oligomer; in some embodiments, for example, the blocking element is a protein that selectively binds to the target sequence and prevents polymerase extension. In embodiments, the blocking element prevents extension during suitable amplification/extension conditions.

Alternate methods for enrichment of fusion-containing templates: Certain amplification reactions conditions may present variable suppression of un-fused templates with the blocking elements described herein, wherein a small proportion of non-fusion amplification product is generated. Alternative approaches that may be implemented, or that may be used in addition to the blocking element, are contemplated herein, and selectively eliminate or render inactive any non-fusion circular templates prior to amplification.

For example, CRISPR-mediated depletion of unwanted target sequences could be performed, wherein a CRISPR-Cas9 complex, for example, using a guide RNA specifically targeting the non-fusion sequence is introduced into a sample containing circularized ssDNA. The CRISPR-Cas9 complex then targets and cleaves the non-fusion sequence present in any circular ssDNA molecules. Following linearization by the CRISPR complex of the non-fusion circular ssDNA molecules, exonuclease digestion could then be performed to digest away the linear ssDNA molecules, enriching for those circular ssDNA molecules containing a fusion gene (e.g., lacking the non-fusion gene sequence targeted by the guide RNA).

Additionally, a biotinylated blocking element could be employed. Following circularization, the biotinylated blocking element is hybridized to the non-fusion gene sequence(s). The circular ssDNA molecules hybridized to the biotinylated blocking elements would then be pulled down using, for example, streptavidin-coated magnetic beads, depleting the sample of any non-fusion containing circular molecules prior to amplification.

As yet another alternative, the blocking oligomer could be used as a splint to enable restriction enzyme-mediated digestion of non-fusion containing circular ssDNA molecules into linear fragments that are not amplifiable. A methylated blocking oligomer could be used in combination with a methylation sensitive restriction enzyme (e.g., NotI, NaeI, NsbI, SalI, HapII, or HaeII).

Sequencing of amplified regions of interest is performed via a next-generation sequencing instrument. In some embodiments, sequencing is accomplished via a single read of greater than about 25 base pairs in length. In other embodiments sequencing is accomplished via paired end reads, where each read within the pair is greater than about 25 bases. Following sequencing, error correction may be performed, and include creating consensus reads from sequences having a shared circularization junction sequence.

A variety of suitable sequencing platforms are available for implementing methods disclosed herein (e.g., for performing the sequencing reaction). Non-limiting examples include SMRT (single-molecule real-time sequencing), ion semiconductor, pyrosequencing, sequencing by synthesis, combinatorial probe anchor synthesis, SOLiD sequencing (sequencing by ligation), and nanopore sequencing. Sequencing platforms include those provided by Singular Genomics™ (e.g., the G4 sequencer), Illumina® (e.g., the HiSeq™, MiSeq™ and/or Genome Analyzer™ sequencing systems); Ion Torrent™ (e.g., the Ion PGM™ and/or Ion Proton™. sequencing systems); Pacific Biosciences (e.g., the PACBIO RS II sequencing system); Life Technologies™ (e.g., a SOLiD sequencing system); Roche (e.g., the 454 GS FLX+ and/or GS Junior sequencing systems). See, for example U.S. Pat. Nos. 7,211,390; 7,244,559; 7,264,929; 6,255,475; 6,013,445; 8,882,980; 6,664,079; and 9,416,409.

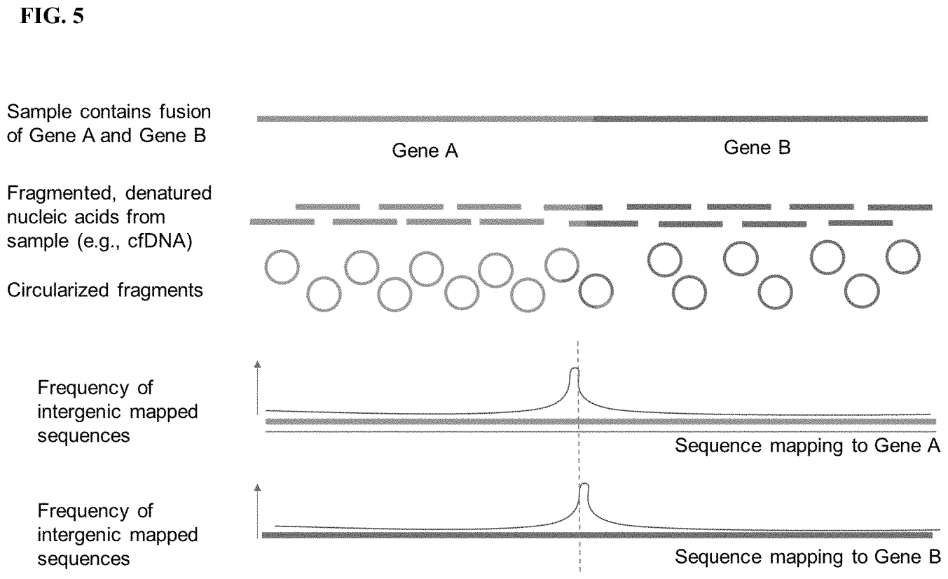

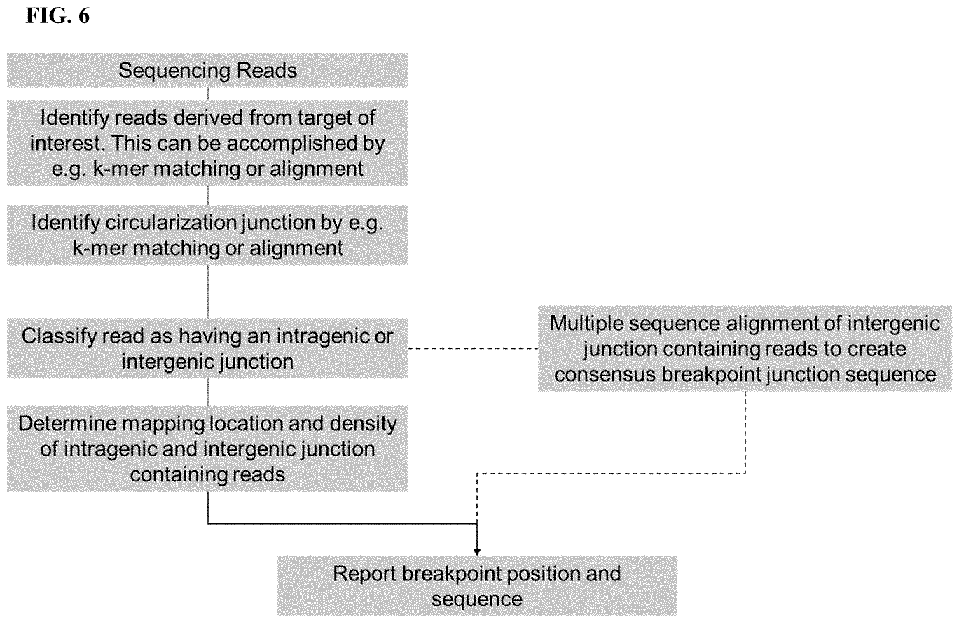

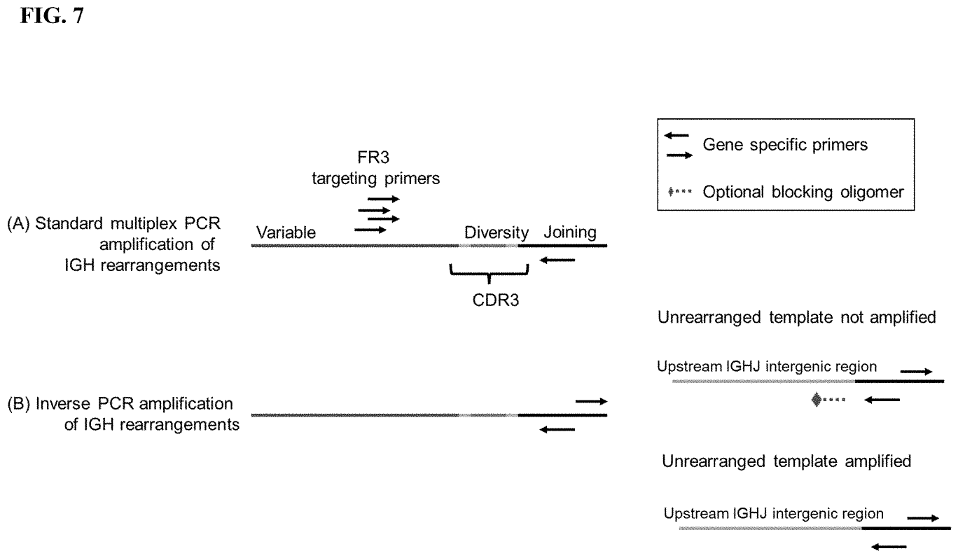

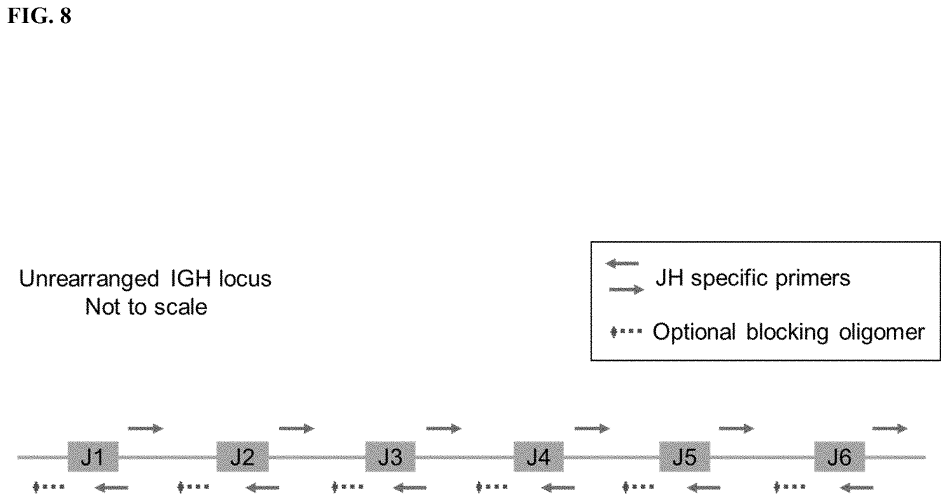

Next, sequence reads are analyzed to assess presence of variants of interest. In some embodiments, this may include use of public software for detecting gene fusions (e.g., GeneFuse; Chen S et al. Int. J. Biol. Sci. 2018; 14(8): 843-848). In other embodiments, this may be accomplished by mapping of reads to a genome and analyzing the localization of reads (e.g., FIG. 5). In yet other embodiments, this may include mapping independent and/or mapping dependent methods, for example those involving the analysis of k-mer substrings (e.g., FIG. 6). FIGS. 7 and 8 provide exemplary bioinformatic workflows for the analysis of rearrangements, translocations, and CNVs using the same method.

Additional fusion detection tools known in the art may be used for analyzing the sequencing reads, such as TRUP (Fernandez-Cuesta, L., Sun, R., Menon, R. et al. Identification of novel fusion genes in lung cancer using breakpoint assembly of transcriptome sequencing data. Genome Biol 16,7 (2015)), chimerascan (Maher C A, Palanisamy N, Brenner JC, Cao X, Kalyana-Sundaram S, Luo S, et al. Chimeric transcript discovery by paired-end transcriptome sequencing. Proc Natl Acad Sci USA. 2009; 106:12353-8), FusionHunter (Li Y, Chien J, Smith DI, Ma J. FusionHunter: identifying fusion transcripts in cancer using paired-end RNA-seq. Bioinformatics. 2011; 27:1708-10), FusionMap (Ge H, Liu K, Juan T, Fang F, Newman M, Hoeck W. FusionMap: detecting fusion genes from next-generation sequencing data at base-pair resolution. Bioinformatics. 2011; 27:1922-8), TopHat-Fusion (Kim D, Salzberg SL. TopHat-Fusion: an algorithm for discovery of novel fusion transcripts. Genome Biol. 2011; 12:R72), defuse (McPherson A, Hormozdiari F, Zayed A, Giuliany R, Ha G, Sun MGF, et al. deFuse: an algorithm for gene fusion discovery in tumor RNA-Seq data. PLoS Comp Biol. 2011; 7:e1001138), SOAPfuse (Jia W, Qiu K, He M, Song P, Zhou Q, Zhou F, et al. SOAPfuse: an algorithm for identifying fusion transcripts from paired-end RNA-Seq data. Genome Biol. 2013;14:R12), FusionSeq (Sboner A, Habegger L, Pflueger D, Terry S, Chen DZ, Rozowsky J S, et al. FusionSeq: a modular framework for finding gene fusions by analyzing paired-end RNA-sequencing data. Genome Biol. 2010; 11:R104), and BreakFusion (Chen K, Wallis JW, Kandoth C, Kalicki-Veizer JM, Mungall KL, Mungall A J, et al. BreakFusion: targeted assembly-based identification of gene fusions in whole transcriptome paired-end sequencing data. Bioinformatics. 2012; 28:1923-4).

Analysis of IGH VDJ rearrangements and translocations: As an exemplary use case, a workflow is presented to achieve targeted amplification of nucleic acids for the simultaneous analysis of IGH V(D)J rearrangements and translocations involving IGHJ genes. Unlike traditional multiplex PCR methods for amplifying VDJ rearrangements, the described method: (1) avoids clone dropout owing to somatic hypermutation within the variable gene region (2) enables detection of IGHJ translocations (3) reduces the number of required primers and (4) enables error correction and template quantitation via analysis of circularization junctions (FIG. 7). Briefly, the workflow begins with extracting sample gDNA using methods known in the art. gDNA molecules may be optionally fragmented to an average length of approximately 200 base pairs, for example if the gDNA is derived from peripheral blood leukocytes or a fresh frozen tumor biopsy. Following fragmentation, templates are circularized via CircLigase™ or analogous method, then IGH rearrangements are selectively amplified using IGHJ targeting primers. Selective amplification may be performed in conjunction with blocking oligomers. As an example, a suitable primer design strategy for selectively amplifying IGHJ rearrangements is presented in FIG. 8.

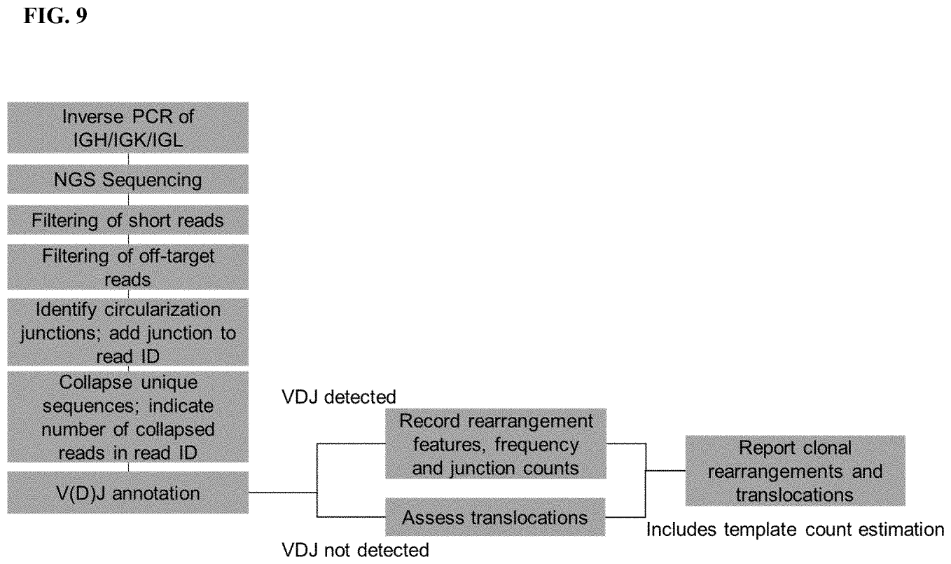

Analysis: FIG. 9. illustrates an overview of the bioinformatics workflow for the analysis of B cell rearrangements via the described method. Amplification of the IGH, IGK and IGL loci is followed by next generation sequencing. Resultant reads are filtered to remove short and off-target products, circularization junctions are identified, unique sequences are collapsed, then annotated for the presence of V(D)J rearrangements via IgBLAST (Ye et al., 2013 doi: 10.1093/nar/gkt382) or similar tool. Reads having a valid V(D)J rearrangement are used to determine the rearrangement frequency and estimate template counts as the number of unique circularization junctions associated with a given rearrangement. The set of identified V(D)J rearrangements is assessed using methods known in the art (e.g. Lay et al., Practical Laboratory Medicine, Volume 22, 2020, e00191) to identify clonal rearrangement markers consistent with the presence of a B cell malignancy. Such markers may be used for longitudinal monitoring of residual disease. Reads lacking an identifiable V(D)J rearrangement are assessed for the presence of translocations using k-mer analysis or methods known in the art (e.g., GeneFuse). Finally, a report is produced indicating the V(D)J clonality of the sample and translocation status, or in the case of residual disease monitoring, whether marker rearrangements are detected in the sample.

Analysis of single cells: The compositions and methods described herein are compatible with common single cell barcoding approaches, allowing for detection of gene fusion events at single cell resolution to potentially reveal clinically relevant tumor heterogeneity. Single cell fusion detection may be part of a broader analysis pipeline to detect and report other cancer variants such as CNVs and SNVs.

Single cell nucleic acid preparation: Target polynucleotides are isolated from a population of cells using methods known in the art. For example, a typical workflow includes the following steps: 1) single cells are individually partitioned into droplets (e.g., sub-nanoliter droplets). 2) Barcoded beads and amplification reagents are introduced. 3) Cell lysis, protease digestion, cell barcoding and targeted amplification occur within the droplets. 4) Droplets are then disrupted, and barcoded DNA is extracted for additional amplification and/or library prep steps. 5) Final libraries are purified and ready for sequencing. A single cell library preparation protocol may also be used, including commercial solutions, for example, those provided by 10× Genomics and/or Mission Bio.

Circularization of nucleic acids from a sample: In circularization, the 5′ end of the nucleic acid molecule is ligated to the 3′ end of the molecule. In an embodiment, a ligase (e.g., CircLigase™ or T4 DNA ligase) is used for circularization of the nucleic acid (DNA or RNA may be circularized). In the case where RNA (e.g., mRNA) is the target for circularization, the RNA is optionally converted to cDNA via reverse transcription. Optionally, following circularization, residual linear molecules may be removed by exonuclease treatment. Additionally, any circularized fragments containing an undesired sequence may be depleted from the pool of circularized fragments, e.g., by hybridization-based pulldown using a probe targeting an undesired sequence, or CRISPR-mediated linearization of circularized fragments containing an undesired sequence, followed by exonuclease treatment (see, for example, U.S. Pat. Pub. 2019/0161752). The use of circularized template material could be advantageous for multiplex PCR, even when used solely in conjunction with traditional inward facing PCR primers, given that the circularized material lacks free 3′ DNA ends that might initiate non-specific amplification. Compared to linear DNA, circularized DNA may enable more on-target amplification when used as a template for inward facing primers and/or outward facing primers in PCR methods.

Sequencing: Amplified nucleic acids are sequenced to determine the presence of one or more gene fusion events. Any suitable commercial sequencing modality may be used, for example in a preferred embodiment, reading the sequence is accomplished using a next-generation sequencing instrument. Reading the sequence can also be accomplished using Sanger sequencing or other low throughput methodologies. The frequency of reads supporting a fusion gene may optionally be compared to those supporting an unfused (i.e., wild type or normal) copy of one or more of the donor or acceptor genes to determine the relative abundance of the gene fusion nucleic acids and whether sufficient read support exists to conclude that a sample contains a gene fusion.

### Example 2. Fusion Detection for Minimal Residual Disease (MRD) Monitoring

The use of standardized multiagent chemotherapy regimens with risk-adapted intensity has greatly contributed to the progressive improvements in survival rates of children with acute lymphoblastic leukemia (ALL). Initial treatment response by serial quantitative measurements of minimal residual disease (MRD) has proven to be one of the strongest independent prognostic factors for pediatric ALL and has been implemented in most treatment protocols currently used. In the Netherlands, MRD monitoring forms the primary basis for risk group stratification since 2004 and is performed using real-time quantitative polymerase chain reaction (RQ-PCR) analysis of rearranged immunoglobulins (IG) and T-cell receptor (TR) genes. The methodology has been highly standardized in international consortia. However, in ^{˜}5% of cases MRD classification is not feasible because a PCR-detectable target cannot be identified or because the target does not reach the required sensitivity (see, Pieters R et al. J. Clin. Oncol. 2016; 34(22):2591-601). In addition, IG/TR rearrangements can be oligoclonal and consequently can be lost during the disease. Consequently, the MRD-based stratification is suboptimal for these patients, with a risk of under- or over-treatment (see, Szczepanski T et al. Blood. 2002; 99(7):2315-23 and van der Velden W H J et al. Leukemia. 2002; 16:928-936). Fusion genes and gene deletions frequently act as primary drivers of leukemogenesis and, as such, can be very stable during disease progression, and suitable as alternative genomic MRD PCR targets. In contrast to fusion transcripts, these genomic fusion breakpoints are independent of gene activity and thus have comparable quantitative dynamics compared to standard IG/TR targets (see, Kuiper R P et al. Br. J. Haematol. 2021; 194(5):888-892, which is incorporated herein by reference in its entirety).

The use of gene fusions or deletions for MRD monitoring requires the identification of the (intronic) genomic breakpoints for these structural variants, which are unique for each patient. These breakpoints can be identified in a direct and unbiased manner based on whole genome sequencing (WGS) data. As described in Example 1, targeted approaches for gene fusion detection enable simplified analysis and reduced cost and have accordingly become a leading approach for clinical applications. The compositions and methods described supra and herein provide sequencing-efficient solutions to achieve targeted sequencing of genetic variations such as SNVs, insertion/deletions, and gene fusions, including those involving novel partners and deriving from novel breakpoints, specifically, for MRD detection. Detailed herein in various embodiments, the method consists of the steps of (1) circularizing nucleic acids derived from a sample; (2) amplifying circularized nucleic acids deriving from one or more targets of interest; and (3) analyzing the amplified fragments via next generation sequencing (NGS).

A method termed the well occupancy method was recently described for estimating the absolute abundance of individual T cell clones or B cell clones and/or nucleic acids encoding individual TCRs and/or IGs among a large number (see, U.S. Pat. No. 10,246,701, which is incorporated herein by reference in its entirety). Briefly, 10,000 PBMC's were allocated to each well of a 96-well plate. Amplification and assignment of well-specific barcodes (which are incorporated into each amplicon by PCR and tailing primers) were performed in each well, then the amplified molecules were sequenced together and the sequence reads were matched back to the starting well based on barcodes. Then, it was determined whether each unique sequence (having a particular CDR3 sequence) was present or absent in each well, such that each unique CDR3 sequence was assigned a pattern of well occupancies. For each individual CDR3 sequence, the occupancy-based method was used to obtain maximum-likelihood estimates of the number of molecules in the original sample; these estimates were determined based solely on the number of wells in which that immune receptor sequence was found. Thus, for each individual unique adaptive immune receptor sequence observed, it was determined the number of containers in which the particular biological sequence was found.

The method described herein for detecting gene fusions via circularization and inverse PCR primers may be applied using such a well occupancy method. Briefly, 10,000 PBMC's (e.g., PBMCs retrieved from a patient for use in MRD detection) are allocated to each well of a 96-well plate. Amplification using inverse PCR primers as described herein is performed, and assignment of well-specific barcodes (which are incorporated into each amplicon by PCR and tailing primers) were performed in each well, then the amplified molecules are sequenced together and the sequence reads matched back to the starting well based on barcodes. Then, it is determined whether each unique sequence (e.g., having a particular gene fusion sequence, such as an IGH locus) is present or absent in each well, such that each unique IGH locus sequence is assigned a pattern of well occupancies. Based on the presence and/or absence of the unique gene fusion sequence, a determination of MRD can be made. Combining the methods described herein with the occupancy-based method may result in significantly higher MRD detection frequencies, e.g., with a lower limit of detection that in traditional practice (e.g., most studies define MRD positivity at 0.01%, which is the detection limit of routine tests, as described in Rocha J M C et al. Mediterr. J. Hematol. Infect. Dis. 2016; 8(1): e2016024, which is incorporated herein by reference).

Circularization of nucleic acids from a sample: In circularization, the 5′ end of the nucleic acid molecule is ligated to the 3′ end of the molecule. In an embodiment, a ligase (e.g., CircLigase™ or T4 DNA ligase) is used for circularization of the nucleic acid (DNA or RNA may be circularized). In the case where RNA (e.g., mRNA) is the target for circularization, the RNA is optionally converted to cDNA via reverse transcription. Optionally, following circularization, residual linear molecules may be removed by exonuclease treatment. Additionally, any circularized fragments containing an undesired sequence may be depleted from the pool of circularized fragments, e.g., by hybridization-based pulldown using a probe targeting an undesired sequence, or CRISPR-mediated linearization of circularized fragments containing an undesired sequence, followed by exonuclease treatment (see, for example, U.S. Pat. Pub. 2019/0161752). The use of circularized template material could be advantageous for multiplex PCR, even when used solely in conjunction with traditional inward facing PCR primers, given that the circularized material lacks free 3′ DNA ends that might initiate non-specific amplification. Compared to linear DNA, circularized DNA may enable more on-target amplification when used as a template for inward facing primers and/or outward facing primers in PCR methods.

Sequencing: Amplified nucleic acids are sequenced to determine the presence of one or more gene fusion events. Any suitable commercial sequencing modality may be used, for example in a preferred embodiment, reading the sequence is accomplished using a next-generation sequencing instrument. Reading the sequence can also be accomplished using Sanger sequencing or other low throughput methodologies. The frequency of reads supporting a fusion gene may optionally be compared to those supporting an unfused (i.e., wild type or normal) copy of one or more of the donor or acceptor genes to determine the relative abundance of the gene fusion nucleic acids and whether sufficient read support exists to conclude that a sample contains a gene fusion.

FIG. 10 illustrates the temporal aspects of MRD testing for acute lymphoblastic leukemia (ALL). Each line represents the level of residual disease over time for a different hypothetical patient following therapeutic intervention (e.g., radiation and/or chemotherapy) at various time points for post-treatment monitoring. The response curves include DP (disease persistence), VEP (very early relapse), ER (early relapse), LR (late relapse), VLR (very late relapse), and NR (no relapse). 10⁻² is denoted as the proportion of leukemic cells which represents the approximate lower limit of detection for VER. Submicroscopic disease detection (i.e., MRD) can typically detect cases of VER, ER, and LR, with a range in the proportion of leukemic cells from about 10⁻² to about 10⁻⁵. Existing methods are largely limited to detecting about 10⁻⁶ leukemic cells in a sample, which may not be sufficient for a patient that will succumb to VLR. The methods described herein allow for detections as low as 10⁻⁵ to 10⁻⁷, benefiting all therapeutic scenarios and benefiting detection in all cases.

The methods described herein enable one to detect malignancy associated markers at all frequencies (e.g., over all ranges from about 10⁻² to about 10⁻⁷), in a sequencing efficient manner, making it suitable for both disease diagnosis and MRD analysis. An additional advantage of the methods described herein over existing commercial solutions, including ClonoSeq® (i.e., kits offered by Adaptive Biotechnology, Inc.) and LymphoTrack® (kits offered by InvivoScribe, Inc.), is that the methods described herein are able to simultaneously evaluate IGH, IGK and IGL locus rearrangements in a single reaction. Existing solutions require separate multiplex PCR reactions, for example, for IGH, IGK and IGL. The need for split PCR reactions increases testing complexity, cost, and time associated with each diagnostic.

### Example 3. T-Cell Receptor Convergence as a Biomarker

Adaptive immune response includes selective response of B and T cells recognizing antigens. The immunoglobulin genes encoding antibody (Ab, in B cell) and T-cell receptor (TCR, in T cell) antigen receptors include complex loci wherein extensive diversity of receptors is produced as a result of recombination of the respective variable (V), diversity (D), and joining (J) gene segments, as well as subsequent somatic hypermutation events during early lymphoid differentiation. Upon TCR engagement by cognate antigens, T lymphocytes up-regulate a number of activation markers and develop multiple effector functions, including proliferation, cytotoxicity, and cytokine production. Knowledge of TCR amino acid sequence enables tracking of specific T cell clones in circulation and peripheral tissues, which significantly contributes to monitoring of, for example, virus-specific T cell immunity and enables differential diagnosis and targeted therapy of T cell-related disorders. Thus, comprehensive assessment of the clonal composition of antigen-specific T cells can deliver important information on cellular immunity in the context of vaccination, tumor control or viral diseases and is of great importance for the clinical evaluation and management (see. e.g., Dziubianau M et al. Am. J. Transplant. 2013; 13(11): 2842-54).

Existing NGS methods for identifying TCR sequences include those that rely on comparing each sequencing read against, for example, Vβ- and Jβ-reference sequences. Alternatively, antigen specific TCR convergence may be determined, which does not require the use of large databases to decode the TCR. This approach relies upon observing TCRs that are similar or identical at an amino acid level, but different at a nucleotide level, indicating that multiple T cell clones independently underwent VDJ recombination and expanded in response to a common antigen. Observing TCR convergence is an indication that the given TCRs are likely to be responding to an antigen that has been presented over an extended period of time, giving different T cell clones the opportunity to independently proliferate in response to the antigen. In the context of cancer, convergent TCRs may be enriched for those that recognize tumor antigens. In a study examining dendritic cell therapy for melanoma, for example, it was observed that the frequency of convergent TCRs at baseline was highly predictive of therapeutic response (see, Storkus W J et al. J. Immunother. Cancer. 2021; 9(11): e003675, which is incorporated herein by reference in its entirety). Similar findings have been reported (see, Naidus E et al. Cancer Immunol. Immunother. 2021; 70(7): 2095-2102) where peripheral blood TCR convergence was directly correlated to patient outcome after PD-L1 blockade in patients with advanced-stage non-small cell lung cancer. The data from these studies suggest that TCR convergence in peripheral blood T cells may represent an actionable biomarker for (1) identification of patients most likely to respond to immunotherapeutic interventions that mechanistically require T cell responses to achieve preferred clinical outcomes and (2) effective longitudinal monitoring of therapeutically meaningful T cell responses in patients on-treatment.

As used herein, a “convergent TCR group” is a set of T cell receptors (TCRs) that are similar in amino acid sequence and functionally equivalent, or are identical or assumed to be identical in amino acid sequence. It is generally assumed, owing to the amino acid similarity, that a convergent TCR group recognizes the same antigen. In some embodiments, convergent TCR group members are identical or assumed to be identical in the variable gene and CDR3 amino acid sequence despite having a different nucleotide sequence. Convergent TCR group members may result from differences in non-templated nucleotide bases at the VDJ junction that arise during the generation of a productive TCR gene rearrangement.

Provided herein are methods for performing a multiplex amplification reaction to amplify target immune receptor nucleic acid template molecules (e.g., TCR molecules) derived from a biological sample, wherein the multiplex amplification reaction includes a plurality of amplification primer pairs including a plurality of junction (J) gene primers directed to a majority of J genes of the target immune receptor, thereby generating target immune receptor amplicon molecules including the target immune receptor repertoire. Using the methods described herein and in Example 1, and outward facing J gene targeting primers, the development of TCRs at baseline and in response to an antigen may be evaluated. To evaluate TCR convergence, for example, instances where TCRβ chains are identical in amino acid sequence but have distinct nucleotide sequences are determined.

Such methods further include performing sequencing of the target immune receptor repertoire amplicons; identifying immune receptor clones from the sequencing and identifying convergent immune receptor clones among the immune receptor clones, wherein the convergent immune receptor clones have a similar or identical amino acid sequence and a different nucleotide sequence; and determining the frequency of convergent immune receptor clones in the sample. Subsequent clinical decision-making may then incorporate the information gained regarding TCR convergence and potential therapeutic avenues to pursue. Additional TCR convergence analysis methodology is described elsewhere, for example, in U.S. Pat. Pub. 2021/0108268, which is incorporated herein by reference in its entirety. These methods provide an efficient way to determine TCR convergence using multiplexed primers, for example outward facing primers as described herein, and allow for the determination of T cell clone VDJ recombination and expansion in response to a common antigen across multiple independent T cell clones.

### Example 4. Determining Blocking Oligomer Efficiency

Following the methods described herein and in Example 1, the efficiency of a blocking oligomer targeting a region of an unrearranged IGHJ6 region was determined. FIG. 13 shows the results of blocking element efficiency as determined by gel electrophoresis analysis. Synthetic oligomers were produced to represent an IGH rearrangement (Fusion, F) and an unrearranged IGHJ6 gene (Wild Type, W). PCR amplification of each template was conducted using inverse PCR primers in the presence or absence of a non-extendable blocking oligomer (denoted by +/−) capable of hybridizing to the W template but not the F template (a blocking oligomer as illustrated in FIG. 1). PCR amplification products were then visualized on an agarose gel. In the absence of the blocking oligomer an equivalent amount of product is observed for the Fusion and Wild Type templates. As expected, addition of the blocker selectively reduces product from the Wild Type template.

### Example 5. Detection of Breakpoint Regions

Gene fusions are an important type of genetic aberration in cancer with relevance to therapy selection and as a marker for measurable residual disease (MRD) monitoring. Traditional multiplex PCR (mPCR) is unable to detect gene fusions with novel partners or breakpoints. Here we introduce a novel mPCR technology for the targeted detection of gene fusions, including those with unknown partners or breakpoints. Using the Singular Genomics G4™ sequencing platform, we applied the methods described herein to simultaneously identify clinically relevant translocations and V(D)J rearrangements of the IGH locus from highly degraded material.

DNA Fragmentation and Circularization: The method begins with a highly efficient intramolecular ligation of DNA fragments followed by a multiplex inverse PCR that preferentially amplifies breakpoint junction containing fragments. To begin, isolated DNA of variable lengths was sheared to approximately 200 bp in length, using either enzymatic fragmentation (e.g., NEBNext dsDNA Fragmentase, catalog #M0348), or manual shearing using the Covaris ME220, followed by QuantaBio sparQ PureMag bead cleanup. Then, 50 ng of the fragmented and bead-purified DNA was heat denatured into single-stranded DNA, followed by circularization using CircLigase™ ssDNA ligase (Lucigen Catalog #CL4111K/CL4115K), using an input of 10 μmol ssDNA per reaction, following the manufacturers protocol. The ssDNA was incubated at 60° C. for 1 hr to circularize the ssDNA, followed by 80° C. for 10 min to inactivate CircLigase™.

After circularization, some un-circularized DNA (both single- and double-stranded) may remain in each sample. A mixture of the enzymes Exonuclease I (NEB) and Exonuclease III (NEB) was used to digest un-circularized DNA by incubating at 37° C. for 1 hr. The remaining circular ssDNA was then purified using a Zymo Oligo Clean & Concentrator Kit.

Inverse PCR: The purified circular ssDNA template was then amplified using inverse PCR as described herein. PCR conditions were adapted from NEB Q5® Polymerase Master Mix reaction conditions, including 0.2 mM dNTPs (each), 0.1 μM primers (each, for example one set of primers 0.1 μM of a first and 0.1 μM of a second primer), 0.2 U/μL Q5 Polymerase, 1 μM of the blocking oligomer (each), and between 500 ng to 2 μg of template. A 2-step amplification protocol was performed, with an initial denaturation step of 96° C., followed by cycling between a 96° C. denaturation step and an annealing/extension step at 62° C. Samples were then taken through library prep. For simplicity, the data in Table 1 was generated with a single pair of joining gene inverse PCR primers and a single blocker. The completed assay (amplifying IGH, IGK, IGL locus rearrangements) will have approximately 22 primers (1F, 6R for IGH locus; 3F, 6R IGK locus; 1F, 5R IGL locus) and 18 different blockers.

Sequencing: Amplicon libraries were sequenced on the G4™ platform via 2×150 bp reads and analyzed to detect translocations. We applied the method to simultaneously detect IGH V(D)J rearrangements and BCL1-JH and BCL2-JH translocations from fragmented IVS-0010 and IVS-0030 reference control gDNA (Invivoscribe Cat #40880550 and 40881750) and healthy donor PBL gDNA.

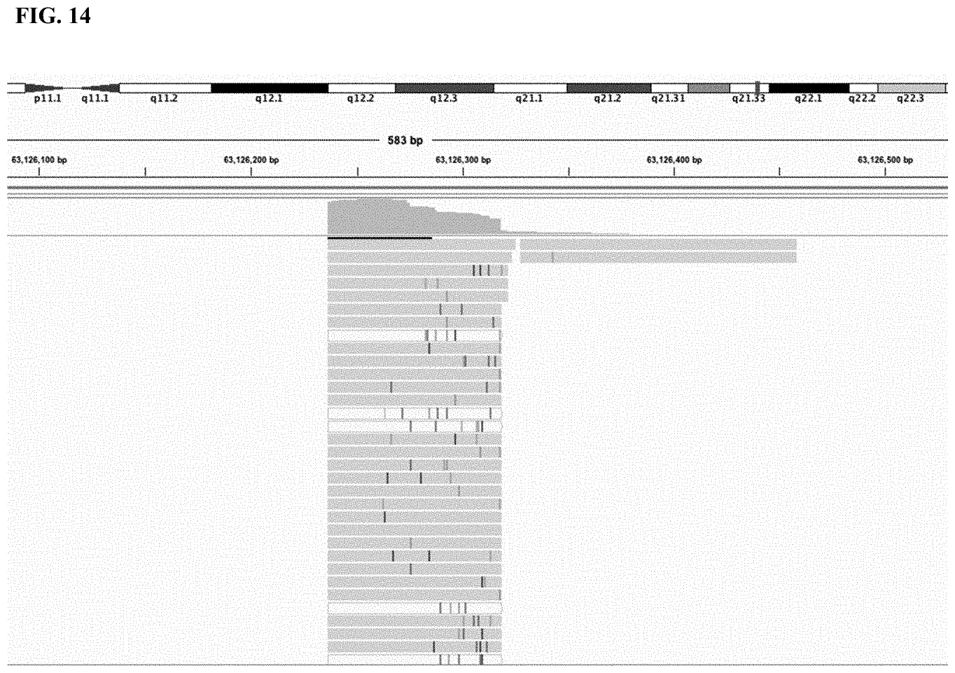

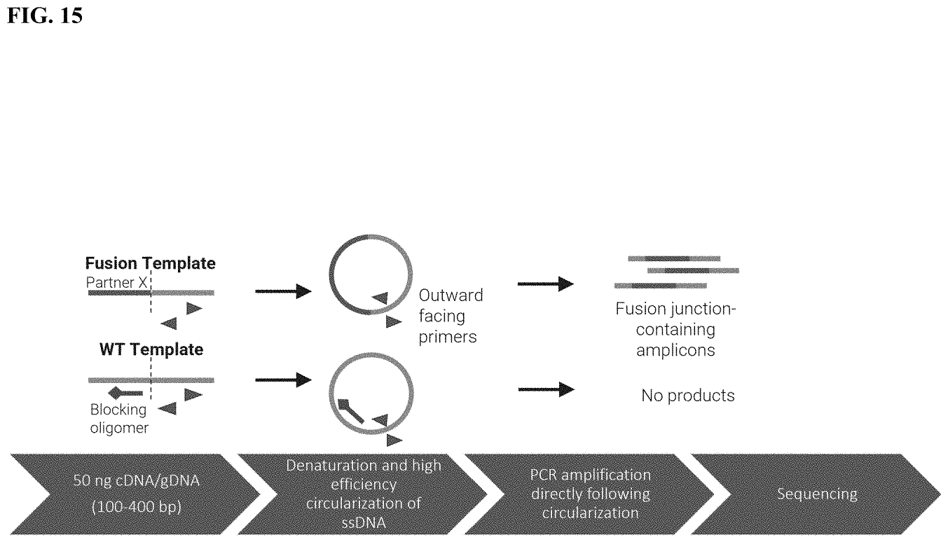

Results: BCL1-JH and BCL2-JH translocations were detected from 50 ng of fragmented gDNA (200 bp avg template length) from IVS-0010 and IVS-0030 reference controls, respectively. Translocations were also detected from 50 ng samples consisting of fragmented reference control material spiked at 1% frequency into a background of fragmented healthy donor PBL. We observe preferential amplification of translocation-containing templates, enabling detection from <1M reads/sample in all conditions tested. V(D)J rearrangements were successfully detected from PBL gDNA using the same multiplex inverse PCR reaction (see, e.g., FIG. 14). A summary of the merged sequencing reads may be found in Table 1.

TABLE 1
The Limit of Detection Analysis from Fragmented Material. For simplicity, the
data in Table 1 were generated with a single pair of joining gene inverse PCR
primers and a single blocker. In embodiments, the complete assay (amplifying IGH,
IGK, IGL locus rearrangements) will have approximately 22 primers (1F, 6R for
IGH locus; 3F, 6R IGK locus; 1F, 5R IGL locus) and 18 blockers. Healthy
donor PBL gDNA and gDNA from IVS-0030 (CAT #: 40881750) was
fragmented to ~200 bp average length via sonication. 50 ng of fragmented
PBL gDNA or 50 ng PBL gDNA spiked with 0.5 ng IVS-0030 was subjected to
circularization and amplification via the assay described herein. Amplicons were
sequenced using 1 × 150 bp reads on the G4 ™. Reads were aligned to the genome
via bwa, then read peaks corresponding to translocation junctions were identified
via MACS2. Unique VDJ rearrangements were identified via IgBLAST. Fraction
on target reads corresponds to reads that map at least in part to the IGH locus.

| | | | Fraction of | Unique VDJ | |
|---|---|---|---|---|---|
| | | | Inserts > | rearrangements | IVS_0030 Detected |
| Sample | Condition | Reads | 100 bp | detected | (BCL2-JH) |
| 1 | 50 ng PBL + | 515,097 | .90 | 40 | Yes |
| | 1% IVS-0030 | | | | chr18:63126274 |
| 2 | 50 ng PBL | 271,183 | .88 | 35 | No |
| 3 | 50 ng PBL + | 91,265 | .89 | 24 | Yes |
| | 1% IVS-0030 | | | | chr18:63126274 |

TABLE 2
Limit of detection analysis using a cell line pool spiked into
healthy donor PBL. Results reflect preparation of libraries
according to the methods described herein from
50 ng gDNA input followed by sequencing to 6M
reads per library according to the methods
described herein. JVM-2 is a lymphoblast cell line from
the peripheral blood of a female subject;
Ramos cells are B lymphocyte cell line that was derived
from a male subject with Burkitt's Lymphoma. Reads were
aligned to the genome via bwa, then read peaks corresponding
to translocation junctions were identified via MACS2.
Unique VDJ rearrangements were identified via
IgBLAST. Fusion marker detection score indicates the
log10(pvalue) for detection of the translocation junction via
MACS2. Results suggest that the assay limit of detection is at
or below 1% when using 50 ng of fragmented gDNA input.

| | Fusion Markers | | | Fraction of |
|---|---|---|---|---|
| | Detection Score − | | VDJ | IGH |
| Marker | Log10(Pval) | | Markers | repertoire |
| Frequency | BCL1-JH | BCL2-JH | Ramos | JVM-2 |
| 25% | 144.91 | 119.47 | 8% | 77% |
| 5% | 102.36 | 36.76 | 4.5% | 77% |
| 1% | 21.81 | 19.23 | 0.14% | 25% |
| 0% | ND | ND | 0% | 0% |

Conclusions: The methods described herein enable rapid mPCR based detection of novel gene fusions from highly degraded material with a sequencing efficiency similar to that of traditional mPCR. As a first application, we have applied these methods to simultaneously detect B cell V(D)J rearrangements and clinically relevant JH translocations from a limited amount of degraded gDNA. In this respect, these methods represent a significant advance over current mPCR based approaches for antigen receptor sequencing. We expect the method to have broad 10 utility for molecular diagnostics and MRD monitoring of disease states, such as cancer. For example, we understand the approach to provide a limit of detection of less than 1%, allowing detection in real patient samples with VAFs (variant allele frequency).

### Embodiments

Embodiment 1. A method for detecting a fusion gene in a sample from a subject, said method comprising: a) circularizing a plurality of linear nucleic acid molecules of the sample to form a plurality of circular template polynucleotides, wherein one or more of the linear nucleic acid molecules comprise a fusion gene, thereby forming one or more fusion gene circular template polynucleotides; b) hybridizing a first primer to a first sequence and a second primer to a second sequence of said one or more fusion gene circular template polynucleotides and extending the first primer and the second primer with a polymerase to generate fusion gene polynucleotide amplification products; and c) detecting the fusion gene polynucleotide amplification products, wherein detecting comprises hybridizing one or more sequencing primers to the fusion gene polynucleotide amplification products and sequencing the fusion gene polynucleotide amplification products.

Embodiment 2. The method of Embodiment 1, wherein the fusion gene is associated with cancer.

Embodiment 3. The method of Embodiment 2, wherein the fusion gene is associated with cancer when said fusion gene occurs at a relative frequency of at least 15% of the total number of fusion gene polynucleotide amplification products.

Embodiment 4. The method of Embodiment 2, wherein the fusion gene is associated with cancer when said fusion gene occurs at a relative frequency of at least 10-5 of the total number of fusion gene polynucleotide amplification products.

Embodiment 5. The method of Embodiment 2, wherein the fusion gene is associated with cancer at a time prior to circularizing a plurality of linear nucleic acid molecules.

Embodiment 6. The method of any one of Embodiments 1 to 5, wherein circularizing comprises contacting the plurality of linear nucleic acid molecules with a ligase capable of template-independent, intra-molecular ligation of linear nucleic acid molecules.

Embodiment 7. The method of any one of Embodiments 1 to 5, wherein circularizing comprises contacting the plurality of linear nucleic acid molecules with a ligase capable of intra-molecular ligation of linear 500 bp or less nucleic acid molecules.

Embodiment 8. The method of Embodiments 6 or 7, wherein the ligase is a pre-adenylated ligase.

Embodiment 9. The method of Embodiments 6 or 7, wherein the ligase is a TS2126 RNA ligase.

Embodiment 10. The method of any one of Embodiments 1 to 9, wherein sequencing comprises (a) extending a sequencing primer by incorporating a labeled nucleotide or labeled nucleotide analogue and (b) detecting the label to generate a signal for each incorporated nucleotide or nucleotide analogue.

Embodiment 11. The method of any one of Embodiments 1 to 10, wherein one or more of the linear nucleic acid molecules do not comprise the fusion gene and circularizing forms one or more non-fusion circular template polynucleotides.

Embodiment 12. The method of Embodiment 11, further comprising hybridizing a third primer and a fourth primer to said one or more non-fusion circular template polynucleotides and extending with a polymerase to generate non-fusion polynucleotide amplification products.

Embodiment 13. The method of Embodiment 12, wherein detecting further comprises comparing the quantity of fusion gene amplification products and the quantity of non-fusion polynucleotide amplification products.

Embodiment 14. The method of any one of Embodiments 11 to 13, comprising binding a blocking element to said one or more non-fusion circular template polynucleotides.

Embodiment 15. The method of Embodiment 14, wherein binding said blocking element comprises binding the blocking element upstream of the first primer.

Embodiment 16. The method of Embodiments 14 or 15, wherein the blocking element binds about 1 to 150 nucleotides upstream relative to the first primer.

Embodiment 17. The method of any one of Embodiments 12 to 16, wherein the quantity of fusion gene amplification products is about 1%, about 5%, about 10%, about 15%, about 20%, about 25%, about 30%, about 40%, about 50%, or about 75% more than the quantity of non-fusion polynucleotide amplification products.

Embodiment 18. The method of any one of Embodiments 12 to 16, wherein the quantity of fusion gene amplification products is about 2-fold, at least about 1.5-fold, at least about 2.0-fold, at least about 2.5-fold, at least about 5-fold, at least about 10-fold, or more than about 10-fold the quantity of non-fusion polynucleotide amplification products.

Embodiment 19. The method of any one of Embodiments 1 to 18, wherein the one or more linear nucleic acid molecules comprise DNA, RNA, or cDNA; optionally wherein the DNA or the RNA are cell-free nucleic acid molecules.

Embodiment 20. The method of any one of Embodiments 1 to 19, wherein the one or more linear nucleic acid molecules comprise cfDNA or isolated DNA, from a formalin fixed paraffin-embedded (FFPE) sample.

Embodiment 21. The method of any one of Embodiments 1 to 20, wherein the first primer and the second primer hybridize to complementary sequences of the one or more fusion gene circular template polynucleotides, wherein the first primer and the second primer are separated by about 1 to about 50 nucleotides.

Embodiment 22. The method of any one of Embodiments 1 to 20, wherein the first primer and the second primer are outward facing primers.

Embodiment 23. The method of any one of Embodiments 1 to 22, wherein the fusion gene comprises a sequence encoding for a B cell receptor.

Embodiment 24. The method of any one of Embodiments 1 to 22, wherein the fusion gene comprises an IGH locus or a BCL-1, BCL-2, BCL-3, or BCL6 locus.

Embodiment 25. The method of any one of Embodiments 1 to 22, wherein the fusion gene comprises a sequence encoding for a complementarity-determining region (CDR) of a T cell receptor or a B cell receptor.

Embodiment 26. The method of any one of Embodiments 1 to 22, wherein the fusion gene comprises a sequence encoding for the CDR3 region of a T cell receptor or a B cell receptor.

Embodiment 27. The method of any one of Embodiments 1 to 22, wherein the fusion gene comprises a sequence encoding for a V region or a complement thereof and a J region or a complement thereof.

Embodiment 28. The method of any one of Embodiments 1 to 22, wherein the fusion gene comprises a RBPSM-MET, BCAN-NTRK1, TRIM22-BRAF, KIAA1549-BRAF, FGFR1-TACC1, EWSR1-FLI1, PAX3-FOXO1, ZFTA-RELA, COL3A1-PLAG1, FGFR3-TACC3, or NPM1-ALK fusion gene.

Embodiment 29. The method of any one of Embodiments 2 to 28, wherein the cancer is a lymphoid hematological malignancy, wherein the lymphoid hematological malignancy is selected from the group consisting of acute T-cell lymphoblastic leukemia (T-ALL), acute B-cell lymphoblastic leukemia (B-ALL), multiple myeloma, plasmacytoma, macroglobulinemia, chronic lymphocytic leukemia (CLL), acute lymphoblastic leukemia (ALL), Hodgkins lymphoma, non-Hodgkins lymphoma, cutaneous T-cell lymphoma, mantle cell lymphoma, peripheral T-cell lymphoma, hairy cell leukemia, T prolymphocytic lymphoma, angioimmunoblastic T-cell lymphoma, T lymphoblastic leukemia/lymphoma, peripheral T-cell lymphoma, adult T cell leukemia/lymphoma, mycosis fungoides, Sezary syndrome, T lymphoblastic leukemia, myeloproliferative neoplasm, and myelodysplastic syndrome.

Embodiment 30. The method of any one of Embodiments 2 to 28, wherein the fusion gene is associated with a pediatric cancer.

Embodiment 31. The method of any one of Embodiments 2 to 30, wherein the cancer is a pilocytic astrocytoma, Ewing sarcoma, supratentorial ependymoma, infantile fibrosarcoma, cholangiocarcinoma, infantile spindle cell sarcoma, infiltrating glioma, ganglioglioma, or acute lymphocytic leukemia.

Embodiment 32. The method of any one of Embodiments 1 to 31, wherein said sample comprises at least 100,000 T cells or B cells.

Embodiment 33. The method of any one of Embodiments 1 to 32, wherein the sample is frozen tissue, formalin-fixed paraffin-embedded (FFPE) tissue, peripheral blood, bone marrow, or cerebral spinal fluid.

Embodiment 34. The method of any one of Embodiments 1 to 33, wherein the sample is obtained pre-treatment or post-treatment.

Embodiment 35. The method of any one of Embodiments 1 to 33, wherein a plurality of samples are obtained at two or more time points.

Embodiment 36. The method of any one of Embodiments 1 to 35, wherein the linear nucleic acid molecules are about 50 to about 1000, 100 to about 800, or about 200 to about 600 nucleotides in length.

Embodiment 37. The method of any one of Embodiments 11 to 36, further comprising: d) amplifying said one or more non-fusion circular template polynucleotides to generate a third number of non-fusion polynucleotide amplification products; and amplifying said one or more fusion circular template polynucleotides to generate a fourth number of fusion polynucleotide amplification products, wherein said third number and said fourth number are substantially the same.

Embodiment 38. The method of Embodiment 37, wherein amplifying said one or more non-fusion circular template polynucleotides comprises hybridizing a third primer and a fourth primer to said one or more non-fusion circular template polynucleotides and extending both primers with a polymerase, and wherein amplifying said one or more fusion circular template polynucleotides comprises hybridizing a third primer and a fourth primer to said one or more fusion circular template polynucleotides and extending both primers with a polymerase.

Embodiment 39. The method of Embodiment 38, wherein the third primer hybridizes upstream of a target sequence, and the fourth primer hybridizes downstream of a target sequence, wherein said target sequence comprises a single-nucleotide variant, an insertion, a deletion, an internal tandem duplication, or a copy number variant.

What is claimed is:

1. A method for detecting a fusion gene in a sample from a subject, said method comprising:
circularizing a fusion linear nucleic acid molecule of the sample to form a fusion gene circular template polynucleotide, wherein said fusion linear nucleic acid molecule comprises a fusion gene; circularizing a non-fusion linear nucleic acid molecule of the sample to form a non-fusion circular template polynucleotide, wherein said non-fusion linear nucleic acid molecule does not comprise the fusion gene;
binding a blocking element to said non-fusion circular template polynucleotide;
hybridizing a first primer to said fusion gene circular template polynucleotide and extending the first primer with a polymerase thereby generating a first fusion extension product;
hybridizing a second primer to said first extension product and extending said second primer with a polymerase thereby generating a second extension product;
sequencing the second extension product, or a complement thereof, thereby detecting the fusion gene.

2. The method of claim 1, wherein binding said blocking element comprises binding the blocking element upstream of the first primer, wherein the blocking element binds about 1 to 150 nucleotides upstream relative to the first primer.

3. The method of claim 2, wherein said blocking element is an oligonucleotide.

4. The method of claim 1, further comprising amplifying the first extension product and/or the second extension product prior to sequencing, wherein amplifying comprises thermal bridge polymerase chain reaction (t-bPCR) amplification, chemical bridge polymerase chain reaction (c-bPCR) amplification, isothermal bridge amplification, chemical-thermal bridge polymerase chain reaction (cT-bPCR) amplification), rolling circle amplification (RCA), exponential rolling circle amplification (eRCA), recombinase polymerase amplification (RPA), or helicase dependent amplification (HDA).

5. The method of claim 1, wherein said fusion linear nucleic acid molecule is genomic DNA.

6. The method of claim 1, wherein said fusion linear nucleic acid molecule is a cell-free nucleic acid molecule.

7. The method of claim 1, wherein said fusion linear nucleic acid molecule is derived from a formalin fixed paraffin-embedded (FFPE) sample.

8. The method of claim 1, wherein said fusion linear nucleic acid molecule is derived from Hodgkin and Reed-Sternberg (HRS) cells.

9. The method of claim 1, wherein said sample is frozen tissue, formalin-fixed paraffin-embedded (FFPE) tissue, peripheral blood, bone marrow, or cerebral spinal fluid.

10. The method of claim 1, wherein circularizing comprises contacting the plurality of linear nucleic acid molecules with a ligase capable of intra-molecular ligation of linear 500 bp or less nucleic acid molecules.

11. The method of claim 10, wherein the ligase is a pre-adenylated ligase.

12. The method of claim 11, wherein the ligase is a TS2126 RNA ligase.

13. The method of claim 1, wherein sequencing comprises sequencing by synthesis, sequencing by binding, sequencing by ligation, or pyrosequencing.

14. The method of claim 1, wherein sequencing comprises hybridizing a sequencing primer to said second extension product, or a complement thereof (a) extending the sequencing primer by incorporating a labeled nucleotide or labeled nucleotide analogue and (b) detecting the label to generate a signal for each incorporated nucleotide or nucleotide analogue.

15. The method of claim 1, wherein a plurality of samples are obtained at two or more time points.

16. The method of claim 15, wherein a first sample is obtained at a first time and a fusion gene is detected; and a second sample is obtained at a second, different, time and the fusion gene is detected.

17. The method of claim 16, wherein the second time is after said subject received treatment.

18. The method of claim 1, wherein said binding said blocking element comprises forming a CRISPR-Cas9 complex with a guide RNA molecule bound to said non-fusion circular template polynucleotide.

19. A method for detecting a fusion gene in a sample from a subject, said method comprising:
contacting a plurality of circular template polynucleotides with a plurality of primers, and binding a primer to each circular template polynucleotide thereby forming primed circular templates, wherein one or more circular template polynucleotides comprise a fusion gene and one or more circular template polynucleotides do not comprise said fusion gene;
contacting said primed circular templates with a plurality of blocking elements and binding a blocking element to the primed templates that do not comprise said fusion gene, wherein said blocking element is an oligonucleotide;
extending a primer bound to a circular template polynucleotide comprising a fusion gene with a polymerase to generate an extension product comprising a complement of said fusion gene; and
sequencing the extension product, or a complement thereof, thereby detecting the fusion gene.

20. The method of claim 19, wherein said plurality of circular template polynucleotides comprise a first circular template polynucleotide comprising a sequence from an IGH locus, a second circular template polynucleotide comprising a sequence from an IGK locus, and a third circular template polynucleotide comprising a sequence from an IGL locus.


SEQUENCE LISTING (1 sequence)

SEQ ID NO: 1 - DNA, 56 bp, Escherichia coli
  source 1..56: mol_type = other DNA
tatcagcacacaattgcccattatacgcgcgtataatggactattgtgtgctgata